(19) United States
(12) Patent Application Publication
(10) Pub. No.: US 2003/0152939 A1
(43) Pub. Date: Aug. 14, 2003

(54) Novel secreted proteins and polynucleotides encoding them

(76) Inventors: Glennda Smithson, Guilford, CT (US); Bryan D. Zerhusen, Branford, CT (US); Mei Zhong, Branford, CT (US); Nikolai V. Khramtsov, Branford, CT (US); Li Li, Branford, CT (US); Vladimir Y. Gusev, Madison, CT (US); Muralidhara Padigaru, Branford, CT (US); David W. Anderson, Branford, CT (US); Richard A. Shimkets, West Haven, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 10/119,431

(22) Filed: Apr. 9, 2002

Related U.S. Application Data
(60) Provisional application No. 60/282,548, filed on Apr. 9, 2001.

(51) Int. Cl. C12Q 1/68; C07H 21/04; C12N 9/00; C12P 21/02; C12N 5/06

(52) U.S. Cl. 435/6; 435/69.1; 435/320.1; 435/325; 435/183; 536/23.2

(57) ABSTRACT

The present invention provides novel isolated polypeptides, a fragment of any of them, polynucleotides encoding them and antibodies that immunospecifically bind to them. The invention additionally provides several methods in which these proteins, polynucleotides and antibodies are used in methods of detection and methods of treatment are also disclosed. These methods of detection and treatment are directed to a broad range of pathological states.

### RELATED APPLICATIONS

This application claims priority to U.S. Ser. No. 60/103,195, filed Oct. 6, 1998, U.S. Ser. No. 09/412231, filed Oct. 5, 1999, and U.S. Ser. No. 60/282548, filed Apr. 9, 2001, each of which is incorporated herein by reference in its entirety.

### FIELD OF THE INVENTION

The invention relates to polynucleotides and polypeptides encoded by such polynucleotides, as well as vectors, host cells, antibodies and recombinant methods for producing the polypeptides and polynucleotides.

### BACKGROUND OF THE INVENTION

Eukaryotic cells are subdivided by membranes into multiple functionally distinct compartments that are referred to as organelles. Each organelle includes proteins essential for its proper function. These proteins can include sequence motifs often referred to as sorting signals. The sorting signals can aid in targeting the proteins to their appropriate cellular organelle. In addition, sorting signals can direct some proteins to be exported, or secreted, from the cell.

One type of sorting signal is a signal sequence, which is also referred to as a signal peptide or leader sequence. The signal sequence is present as an amino-terminal extension on a newly synthesized polypeptide chain A signal sequence can target proteins to an intracellular organelle called the endoplasmic reticulum (ER).

The signal sequence takes part in an array of protein-protein and protein-lipid interactions that result in translocation of a polypeptide containing the signal sequence through a channel in the ER. After translocation, a membrane-bound enzyme, named a signal peptidase, liberates the mature protein from the signal sequence.

The ER functions to separate membrane-bound proteins and secreted proteins from proteins that remain in the cytoplasm. Once targeted to the ER, both secreted and membrane-bound proteins can be further distributed to another cellular organelle called the Golgi apparatus. The Golgi directs the proteins to other cellular organelles such as vesicles, lysosomes, the plasma membrane, mitochondria and microbodies.

Only a limited number of genes encoding human membrane-bound and secreted proteins have been identified. Examples of known secreted proteins include human insulin, interferon, interleukins, transforming growth factor-beta, human growth hormone, erythropoietin, and lymphokines.

### SUMMARY OF THE INVENTION

The present invention is based, in part, upon the discovery of novel polynucleotides identified as containing presumptive signal sequences. These clones nucleotide sequences have been designated clone NOV1 (SEQ ID NOs: 1, 3, 5), which encodes a protein having sequence similarity to syncollin; clone NOV2 (SEQ ID NO: 7); clone NOV3 (SEQ ID NO: 9, 11, 13), which encodes a protein having similarity to claudin; and clone NOV4 (SEQ ID NO: 14, 16), which encodes a cytokine-like protein. Also provided in the invention are proteins encoded by these sequences. These polypeptides correspond to the amino acid sequences of SEQ ID NOs: 2, 4, 6, 8, 10, 12, 15 and 17, respectively.

The invention includes an isolated nucleic acid molecule which includes a nucleotide sequence at least 85% similar to the nucleotide sequence of SEQ ID NOs: 1, 3, 5, 7, 9, 11, 13, 14 or 16, or a complement thereof.

The invention also includes an isolated polypeptide having an amino acid sequence at least 80% homologous to SEQ ID NOs: 4, 6, 8, or 10, or a fragment having at least 15 amino acids of these amino acid sequences. Also included is a naturally occurring polypeptide variant consisting of the amino acid sequence of SEQ ID NOs. 4, 6, 8, or 10, wherein the polypeptide is encoded by a nucleic acid molecule which hybridizes under stringent conditions to a nucleic acid molecule consisting of SEQ ID NOs: 1, 3, 5, 7, 9, 11, 13, 14 or 16.

Also included in the invention is an antibody which selectively binds to the polypeptide of SEQ ID NOs: 2, 4, 6, 8, 10, 12, 15 or 17.

The invention further includes a method for producing the aforementioned polypeptides by culturing a host cell expressing one of the herein described nucleic acids under conditions in which the nucleic acid molecule is expressed.

The invention also includes methods for detecting the presence of these polypeptides in a sample from a mammal, e.g., a human, by contacting a sample from the mammal with an antibody which selectively binds to one of the herein described polypeptides, and detecting the formation of reaction complexes including the antibody and the polypeptide in the sample. Detecting the formation of complexes in the sample indicates the presence of the polypeptide in the sample.

The invention further includes a method for detecting or diagnosing the presence of a disease associated with altered levels of a polypeptide having an amino acid sequence at least 80% identical to a polypeptide with an amino acid sequence selected from the group consisting of SEQ ID NOs: 2, 4, 6, 8, 10, 12, 15 and 17 in a sample. The method includes measuring the level of the polypeptide in a biological sample from the mammalian subject, e.g., a human, and comparing the level detected to a level of the polypeptide present in normal subjects, or in the same subject at a different time, e.g., prior to onset of a condition. An increase or decrease in the level of the polypeptide as compared to normal levels indicates a disease condition.

Also included in the invention is a method of detecting the presence of a nucleic acid molecule having a sequence at least 80% identical to a nucleic acid comprising a sequence selected from the group consisting of SEQ ID NOs: 1, 3, 5, 7, 9, 11, 13, 14 or 16, or 18, 20, 22, 24 or 26 in a sample from a mammal, e.g., a human. The method includes contacting the sample with a nucleic acid probe or primer which selectively hybridizes to the nucleic acid molecule and determining whether the nucleic acid probe or primer binds to a nucleic acid molecule in the sample. Binding of the nucleic acid probe or primer indicates the nucleic acid molecule is present in the sample.

The invention further includes a method for detecting or diagnosing the presence of a disease associated with altered levels of a nucleic acid at least 80% identical to a nucleic acid comprising a sequence selected from the group consisting of SEQ ID NOs:, 3, 5, 7, 9, 11, 13, 14 or 16, or 18, 20, 22, 24 or 26 in a sample from a mammal, e.g,. a human. The method includes measuring the level of the nucleic acid in a biological sample from the mammalian subject and comparing the level detected to a level of the nucleic acid present in normal subjects, or in the same subject at a different time. An increase or decrease in the level of the nucleic acid as compared to normal levels indicates a disease condition.

The invention also includes a method of treating a pathological state in a mammal, e.g,. a human, by administering to the subject a polypeptide to the subject in an amount sufficient to alleviate the pathological condition. The polypeptide has an amino acid sequence at least 80% identical to a polypeptide with an amino acid sequence selected from the group consisting of SEQ ID NOs: 2, 4, 6, 8, 10, 12, 15 and 17, or a biologically active fragment thereof.

Alternatively, the mammal may be treated by administering an antibody as herein described in an amount sufficient to alleviate the pathological condition.

Pathological states for which the methods of treatment of the invention are envisioned include a cancer, a tumor, an immune disorder, an immune deficiency, an autoimmune disease, acquired immune deficiency syndrome, transplant rejection, allergy, an infection by a pathological organism or agent, an inflammatory disorder, arthritis, a hematopoietic disorder, a skin disorder, atherosclerosis, restenosis, a neurological disease, Alzheimer's disease, peripheral neuropathy, trauma, a surgical or traumatic wound, a spinal cord injury, and a skeletal disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

An examination of nucleic acid sequences identified based on their differential expression in cells revealed six clones with candidate secreted sequences. Of these sequences, four are not previously described. These include clones NOV1, NOV2, NOV3, and NOV4. Two clones, clone FIZZX1 and mamm-x (mammaglobin) corresponded to previously described sequences.

The present invention provides novel nucleotides and polypeptides encoded thereby. Included in the invention are the novel nucleic acid sequences, their encoded polypeptides, antibodies, and other related compounds. The NOV1, NOV2, NOV3 and NOV4 sequences, and all variant thereof, are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 1 provides a summary of the disclosed nucleic acids and their encoded polypeptides.

TABLE 1
Sequences and Corresponding SEQ ID Numbers

| | Internal | | | |
|---|---|---|---|---|
| NOVX | Identi- | SEQ ID NO | SEQ ID NO | |
| Assignment | fication | (nucleic acid) | (amino acid) | Homology |
| NOV1 | NOV1 | 1, 3, 5 | 2, 4, 6 | SYNCOLLIN |
| NOV2 | NOV2 | 7 | 8 | none |
| NOV3 | NOV3 | 9, 11, 13 | 10, 12 | CLAUDIN |
| NOV4 | NOV4 | 14, 16 | 15, 17 | Cytokine |
| FIZZ-X | FIZZX | 18, 20, 22, 24 | 19, 21, 23, | FIZZ-3 |
| | | | 25 | |
| MAMM-X | MAMMX | 26 | 27 | Mammoglobin |

Table 1 indicates homology of NOVX nucleic acids to known protein families. Thus, the nucleic acids and polypeptides, antibodies and related compounds according to the invention corresponding to a NOVX as identified in column 1 of Table 1 will be useful in therapeutic and diagnostic applications implicated in, for example, pathologies and disorders associated with the known protein families identified in column 5 of Table 1.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table 1, the NOVX polypeptides of the present invention show homology to, and contain domains that are characteristic of, other members of such protein families. Details of the sequence relatedness and domain analysis for each NOVX are presented in the Examples.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table 1.

The NOVX nucleic acids and polypeptides are also useful for detecting specific cell types. Details of the expression analysis for each NOVX are presented in the Examples. Accordingly, the NOVX nucleic acids, polypeptides, antibodies and related compounds according to the invention will have diagnostic and therapeutic applications in the detection of a variety of diseases with differential expression in normal vs. diseased tissues, e.g. a variety of cancers.

Additional utilities for NOVX nucleic acids and polypeptides according to the invention are disclosed herein.

NOV1

The membrane proteins synaptobrevin, syntaxin, and SNAP-25 form the core of a ubiquitous fusion machine that interacts with the soluble proteins NSF and alpha-SNAP. During regulated exocytosis, membrane fusion is usually strictly controlled by Ca2+ ions. However, the mechanism by which Ca2+ regulates exocytosis is still unclear. Edwardson et al. (Cell 1997;90:325-33) showed that the membranes of exocrine secretory granules contain an 18-kDa protein, syncollin, that binds to syntaxin at low Ca2+ concentrations and dissociates at concentrations known to stimulate exocytosis. Syncollin has a single hydrophobic domain at its N-terminus and shows no significant homology with any known protein. Recombinant syncollin inhibits fusion in vitro between zymogen granules and pancreatic plasma membranes, and its potency falls as Ca2+ concentration rises. They suggested that syncollin acts as a Ca2(+)-sensitive regulator of exocytosis in exocrine tissues.

The NOV1 clone (also referred to herein as clone 2353875, 2353875f or CG51689-02) was analyzed, and the nucleotide and encoded polypeptide sequences are shown in Table 1A.

TABLE 1A
NOV1 Sequence Analysis

| | |
|---|---|
| SEQ ID NO: 1 | 596 bp |

| | | |
|---|---|---|
| NOV1a, | 1 | ACGCGTGCAGGTGGCACTGCCACCATGTCCCCGCTGCGCCCGCTG |
| 2353875f DNA Sequence | | |
| | 46 | CTGCTGGCCCTGGCCCTTGCCTCCGTGCCTTGCGCCCAGGGCGCC |
| | 91 | TGCCCCGCCTCCGCCGACCTCAAGCACTCGGACGGGACGCGCACT |
| | 136 | TGCGCCAAGCTCTATGACAAGAGCGACCCCTACTATGAGAACTGC |
| | 181 | TGCGGGGGCGCCGAGCTGTCGCTGGAGTCGGGCGCAGACCTGCCC |
| | 226 | TACCTGCCCTCCAACTGGGCCAACACCGCCTCCTCACTTGTGGTG |
| | 271 | GCCCCGCGCTGCGAGCTCACCGTGTGGTCCCGGCAAGGCAAGGCG |
| | 316 | GGCAAGACGCACAAGTTCTCTGCCGGCACCTACCCGCGCCTGGAG |
| | 361 | GAGTACCGCCGGGGCATCTTAGGAGACTGGTCCAACGCTATCTCC |
| | 406 | GCGCTCTACTGCAGGTGCAGCTGATGCATTGCTGGTCTCTCATCT |
| | 451 | GCAGCTTCCACAGAGTGCCAAGCCCCTCACTCACCCATCCCTGGG |
| | 496 | CTCTGCTCCGGGCCCCAAGACCCAGGAGGAGGAGCGTTCTGCCTG |
| | 541 | CCCCCTCCCACCTCCCCTGCAATACAGCCTTTGTGCAGTTGTAAA |
| | 586 | AAAAAAAAAAA |

| | |
|---|---|
| ORF Start: ATG at 25 | ORF Stop: TGA at 426 |

| | | |
|---|---|---|
| SEQ ID NO: 2 | 134 aa | MW at kD |

| | |
|---|---|
| NOV1a, | MSPLRPLLLALALASVPCAQGACPASADLKHSDGTRTCAKLYDKSDPYYENCCGGAELSL |
| 2353875f Protein | |
| Seqence | ESGADLPYLPSNWANTASSLVVAPRCELTVWSRQGKAGKTHKFSAGTYPRLEEYRRGILG |
| | DWSNAISALYCRCS |

| | |
|---|---|
| SEQ ID NO: 3 | 769 bp |

| | | |
|---|---|---|
| NOV1b, | 1 | TTCAACCGCACAAAGGCTGTATTGCAGGGGAGGTGGGAGGGGGCA |
| 2353875 Updated DNA | | |
| Sequence | 46 | GGCAGAACGCTCCTCCTCCTGGGTCTTGGGGCCCCGGAGCAGAGC |
| | 91 | CCAGGGATGGGCTGAGTGAGGGGCTTGGCACTCTGTGGAAGCTGC |
| | 136 | AGATGAGAGACCAGCAATGCATCAGCTGCACCTGCAGTAGAGCGC |
| | 181 | GGAGATAGCGTTGGACCAGTCTCCTAAGATGTCCCCGCTGCGCCC |
| | 226 | GCTGCTGCTGGCCCTGGCCCTTGCCTCCGTGCCTTGCGCCCAGGG |
| | 271 | CGCCTGCCCCGCCTCCGCCGACCTCAAGCACTCGGACGGGACGCG |
| | 316 | CACTTGCGCCAAGCTCTATGACAAGAGCGACCCCTACTATGAGAA |
| | 361 | CTGCTGCGGGGGCGCCGAGCTGTCGCTGGAGTCGGGCGCAGACCT |
| | 406 | GCCCTACCTGCCCTCCAACTGGGCCAACACCGCCTCCTCACTTGT |
| | 451 | GGTGGCCCCGCGCTGCGAGCTCACCGTGTGGTCCCGGCAAGGCAA |
| | 496 | GGCGGGCAAGACGCACAAGTTCTCTGCCGGCACCTACCCGCGCCT |
| | 541 | GGAGGAGTACCGCCGGGGCATCTTAGGAGACTGGTCCAACGCTAT |
| | 586 | CTCCGCGCTCTACTGCAGGTGCAGCTGATGCATTGCTGGTCTCTC |
| | 631 | ATCTGCAGCTTCCACAGAGTGCCAAGCCCCTCACTCAGCCCATCC |
| | 676 | CTGGGCTCTGCTCCGGGGCCCCAAGACCCAGGAGGAGGAGCGTTC |
| | 721 | TGCCTGCCCCCTCCCACCTCCCCTGCAATACAGCCTTTGTGCAGT |
| | 766 | TAAA |

| | |
|---|---|
| ORF Start: ATG at | ORF Stop: at |

| | | |
|---|---|---|
| SEQ ID NO: 4 | 134 aa | MW at kD |

| | |
|---|---|
| NOV1b, | MSPLRPLLLALALASVPCAQGACPASADLKHSDGTRTCAKLYDKSDPYYENCCGGAELSL |
| 2353875 Updated Protein | |
| Sequence | ESGADLPYLPSNWANTASSLVVAPRCELTVWSRQGKAGKTHKFSAGTYPRLEEYRRGILG |
| | DWSNAISALYCRCS |

| | |
|---|---|
| SEQ ID NO: 5 | 867 bp |

| | |
|---|---|
| NOV1c, | TGCATCAGCTGCACCTGCAGTAGAGCGCGGAGATAGCGTTGGACCAGTCTCCTAAGATGC |
| CG51689-02 DNA | |
| Sequence | CCCGGCTGCCACCATGTCCCCGCTGCGCCCGCTGCTGCTGGCCCTGGCCCTTGCCTCCGT |
| | GCCTTGCGCCCAGGGCGCCTGCCCCGCCTCCGCCGACCTCAAGCACTCGGACGGGACGCG |
| | CACTTGCGCCAAGCTCTATGACAAGAGCGACCCCTACTATGAGAACTGCTGCGGGGGCGC |
| | CGAGCTGTCGCTGGAGTCGGGCGCAGACCTGCCCTACCTGCCCTCCAACTGGGCCAACAC |
| | CGCCTCCTCACTTGTGGTGGCCCCGCGCTGCGAGCTCACCGTGTGGTCCCGGCAAGGCAA |
| | GGCGGGCAAGACGCACAAGTTCTCTGCCGGCACCTACCCGCGCCTGGAGGAGTACCGCCG |
| | GGGCATCTTAGGAGACTGGTCCAACGCTATCTCCGCGCTCTACTGCAGGTGCAGCTGATG |
| | CATTGCTGGTCTCTCATCTGCAGCTTCCACAAAGGCTGTATTGCAGGGGAGGTGGGAGGG |
| | GGCAGGCAGAACGCTCCTCCTCCTGGGTCTTGGGGCCCCGGAGCAGAGCCCAGGGATGGG |
| | CTGAGTGAGGGGCTTGGCACTCTGTGGAAGCTGCAGATGAGGGACCAGCAATGCATCAGC |
| | TGCACCTGCAGTAGAGCGCCGAGCTGTCGCTGGAGTCGGGCGCAGACCTGCCCTACCTGC |
| | CCTCCAACTGGGCCAACACCGCCTCCTCACTTGTGGTGGCCCCGCGCTGCGAGCTCACCG |
| | TGTGGTCCCGGCAAGGCAAGGCGGGCAAGACGCACAAGTTCTCTGCCGGCACCTACCCGC |
| | GCCTGGAGGCCTACCCGCGCCTGGAGA |

| | |
|---|---|
| ORF Start: ATG at | ORF Stop: at |

| | | |
|---|---|---|
| SEQ ID NO: 6 | 134 aa | MW at kD |

| | |
|---|---|
| NOV1c, | MSPLRPLLLALALASVPCAQGACPASADLKHSDGTRTCAKLYDKSDPYYENCCGGAELSL |
| CG51689-02 Protein | |
| Sequence | ESGADLPYLPSNWANTASSLVVAPRCELTVWSRQGKAGKTHKFSAGTYPRLEEYRRGILG |
| | DWSNAISALYCRCS |

Sequence comparison of the above protein sequences yields the following sequence relationships shown in Table 1B.

TABLE 1B
Comparison of NOV1a against NOV1b and NOV1c.

| | NOV1a | Identities/ |
|---|---|---|
| Protein | Residues/ | Similarities for |
| Sequence | Match Residues | the Matched Region |
| NOV1b | 1 . . . 134 | 134/134 (100%) |
| NOV1c | 1 . . . 134 | 134/134 (100%) |

Further analysis of the NOV1c protein yielded the following sequence relationships shown in Table 1C.

TABLE 1C
Protein Sequence Properties NOV1c

| | |
|---|---|
| PSort | 0.5613 probability located outside the cell; 0.1000 probability |
| analysis: | located in the endoplasmic reticulum (membrane), endo- |
| | plasmic reticulem (lumen), or the microbody (peroxisome) |
| SignalP | Cleavage site between residues 21 and. 22; i.e. at the dash in |
| analysis: | the sequence AQG-AC |

A search of the NOV1a protein against the Geneseq database, a proprietary database that contains sequences published in patents and patent publication, yielded several homologous proteins shown in Table 1D.

TABLE 1D
Geneseq Results for NOV1a

| | Protein/ | NOV1a | Identities/ | |
|---|---|---|---|---|
| | Organism/ | Residues/ | Similarities | |
| Geneseq | Length | Match | for the | Expect |
| Identifier | [Patent #, Date] | Residues | Matched Region | Value |
| AAB42763 | Human ORFX | 1 . . . 134 | 134/134 (100%) | 3.2e−71 |
| | ORF2527 | 23 . . . 156 | 134/134 (100%) | |
| | polypeptide | | | |
| | sequence SEQ | | | |
| | ID NO:5054— | | | |
| | Homo sapiens, | | | |
| | 156 aa. | | | |
| AAY92233 | Clone | 1 . . . 134 | 134/134 (100%) | 3.2e−71 |
| | NOV1f— | 1 . . . 134 | 134/134 (100%) | |
| | syncollin | | | |
| | homologue— | | | |
| | Homo sapiens, | | | |
| | 134 aa. | | | |
| AAY59896 | Human normal | 2 . . . 134 | 133/133 (100%) | 1.1e−70 |
| | pancreas tissue | 1 . . . 133 | 133/133 (100%) | |
| | derived protein | | | |
| | 4— | | | |
| | Homo sapiens, | | | |
| | 133 aa. | | | |
| AAB54267 | Human | 2 . . . 134 | 133/133 (100%) | 1.1e−70 |
| | pancreatic | 2 . . . 134 | 133/133 (100%) | |
| | cancer antigen | | | |
| | protein | | | |
| | sequence SEQ | | | |
| | ID NO: 719— | | | |
| | Homo sapiens, | | | |
| | 134 aa. | | | |
| ABG09717 | Novel human | 2 . . . 134 | 131/133 (98%) | 3.3e−69 |
| | diagnostic | 1 . . . 133 | 131/133 (98%) | |
| | protein | | | |
| | #9708— | | | |
| | Homo sapiens, | | | |
| | 133aa | | | |

In a BLAST search of public sequence databases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| Q8VCK7 | RIKEN CDNA | 1 . . . 133 | 99/133 (74%) | 7.8e-52 |
| | 0910001K16 | 1 . . . 133 | 111/133 (83%) | |
| | GENE—Mus | | | |
| | musculus | | | |
| | (Mouse), 134 aa. | | | |
| Q9DC81 | 0910001K16RIK | 1 . . . 133 | 98/133 (73%) | 2.1e−51 |
| | PROTEIN— | 1 . . . 133 | 111/133 (83%) | |
| | Mus musculus | | | |
| | (Mouse), 134 aa | | | |
| O35775 | Syncollin | 1 . . . 133 | 96/133 (72%) | 8.9e−51 |
| | (SIP9)— | 12 . . . 144 | 111/133 (83%) | |
| | Rattus norvegicus | | | |
| | (Rat), 145 aa | | | |
| Q924N6 | AGRIN— | 2 . . . 27 | 16/28 (57%) | 0.39 |
| | Mus musculus | 10 . . . 37 | 18/28 (64%) | |
| | (Mouse), 69 aa | | | |
| | (fragment). | | | |
| Q10731 | Fungal protease | 8 . . . 53 | 15/46 (32%) | 0.50 |
| | inhibitor F | 12 . . . 49 | 26/46 (56%) | |
| | precursor | | | |
| | (FPI-F)— | | | |
| | Bombyx mori | | | |
| | (Silk moth), | | | |
| | 77 aa. | | | |

Pfam analysis of NOV1 predicted no significant homology to known domains. Further analysis of NOV1 is provided in the Examples.

NOV2

NOV2 (also referred to as) is 876 nucleotides in length and includes an open reading frame encoding a secreted protein from nucleotides 129 to 533. The NOV2 nucleotide sequence is shown in Table 2A ( SEQ ID NO: 7), along with an encoded polypeptide of 135 amino acids (SEQ ID NO: 8). No significant homology was found when the NOV2 nucleotide sequence was searched against other sequences in the GenBank database using BLASTP search protocols. Clone NOV2 was isolated from human testis.

The NOV2 clone (also referred to herein as Clone NOV2) was analyzed, and the nucleotide and predicted polypeptide sequences are shown in Table 2A.

TABLE 2A
NOV2 Sequence Analysis

| | |
|---|---|
| SEQ ID NO: 7 | 876 bp |

| | |
|---|---|
| NOV2, | AGAAGCTAGAATTGGACAGGTGGGCTCTCTGATTTAAAGTAAGTA |
| NOV2f DNA Sequence | |
| | CATGAGAAAGTTAAGGCCACAATTCATGGATAGTGAAATCTGTAT |
| | CCACAATTCATTTTCATCAAGTCAGAAAGTTATCAAACATGGAAT |
| | TCTGCACTTTTGTTATGGCATCAAGCTTCCAAAGCAACACAGTAA |
| | TAGTCATACACTTCAACAACCACCAACACTGTGTCAGATGTCCAC |
| | TCTTCAAGTATGAAACTTGTGCAGTAGCATGGCTTCTACCTGGTG |
| | GCAAGAACTTAGTAATCAACATTACTGATACCCCTGTCACTACTG |
| | ATATCTGGAGGGCCTATTTCTTCAGAATTATTCTCCAGAGAAAAC |
| | ACTTTCAAACTCACACTGAGGTGCAAGTGATGTGTCCTCACGTAA |
| | CAGAGCAAACTAAAAACTCAACTGAAATAGAGTATTCATTTAGTA |
| | TTTATGGTCAGGAGGATGGCGTAAAGATCACTCCATCTTGTGGAT |
| | CCCATCCTTGCTATGCCACGTGGATCGAATGTCATGTTTAAAAAG |
| | TGGTCAAGTCTACTCTTTAGTTCTTCAAAAGCAAGGACACAGCAG |
| | GTCATTGGACACGTCTGCCACAGGCCACATCAGTTCTCCCTCTTT |
| | CAAGCTCCAAGTCTGAGTCATTCAGGCCAGTGTTCTGCAACACAA |
| | ACAGGCATCTGTGGAAGAGCTAGTATGGTGTTGGTATCAGTCACA |
| | TTCAGGGTTGGAGAAATCTGTGCACTGGAAGCTTGAGTATTCAGG |
| | GAGGAAAGGAGAGAAAAGGACATAGAGTAGACTGAAGACAGGTTA |
| | TTACACTGAAAACAACTAAGGAAAGTATCAGCCAGGCGGGGTACC |
| | TATAATCCCAGCACTTTGCAA |

| | |
|---|---|
| | ORF Stop: TAA |
| ORF Start: ATG at 129 | at 533 |
| SEQ ID NO: 8 | 135 aa |

| | |
|---|---|
| NOV2, | MetGluPheCysThrPheValMetAlaSerSerPheGlnSerAsn |
| NOV2f Protein Sequence | |
| | ThrValIleValIleHisPheAsnAsnHisGlnHisCysValArg |
| | CysProLeuPheLysTyrGluThrCysAlaValAlaTrpLeuLeu |
| | ProGlyGlyLysAsnLeuValIleAsnIleThrAspThrProVal |
| | ThrThrAspIleTrpArgAlaTyrPhePheArgIleIleLeuGln |
| | ArgLysHisPheGlnThrHisThrGluValGlnValMetCysPro |
| | HisValThrGluGlnThrLysAsnSerThrGluIleGluTyrSer |
| | PheSerIleTyrGlyGlnGluAspGlyValLysIleThrProSer |
| | CysGlySerHisProCysTyrAlaThrTrpIleGluCysHisVal |

Further analysis of the NOV2 protein yielded the following properties shown in Table 2B.

TABLE 2B
Protein Sequence Properties NOV2

| | |
|---|---|
| Psort analysis: | likely located in the mitochondrial matrix space or in |
| | the microbody (peroxisome) |
| SignalP analysis: | No Known Signal Sequence Predicted |

PFam analysis predicts that the NOV2 protein contains the domains shown in the Table

TABLE 2C
Domain Analysis of NOV2

| | | Identities/ | |
|---|---|---|---|
| | NOV2 | Similarities for | Expect |
| Pfam Domain | Match Region | the Matched Region | Value |
| RFAG_ECOLI | 76 . . . 116 | 17/42 (40%) | 0.056 |
| | | 26/42 (61%) | |

The nucleotide sequence and amino acid sequence for NOV2 was searched against other databases using SignalPep and PSort search protocols. NOV2 apparently has no amino terminal signal peptide and is likely located in the mitochondrial matrix space or in the microbody (peroxisome). Further analysis of NOV2, if any, is presented in the Examples.

NOV3 NOV3—Clone NOV3 (CLAUDIN HOMOLOG)

The NOV3 clones (also referred to as NOV3 or CG52234-02) were analyzed, and the nucleotide and predicted polypeptide sequences are shown in Table 3A.

TABLE 3A
NOV3 Sequence Analysis

| | |
|---|---|
| SEQ ID NO: 9 | 1530 bp |

| | |
|---|---|
| NOV3a, | GCCGGTCTGGCCCGGATCAGGGAGTCCTTCTGCTCCCTGGCACGG |
| NOV3 DNA | |
| Sequence | CTCTGCGCTGAACCCACCCGGCCTGCGGAGAGCAGACAAGTGCCT |
| | CTTGGGCCCGCTTCTCTAACAAATGTAAAAATAATGCCCTTGAAC |
| | CAGGAGCGAAACTGAGCTATCTAAGGAAAACACTGTGAGCAAATA |
| | CTGAGAGCCTAGGGAAACCATCTGATTAGAAGAGCTCCCCTCAGG |
| | AGCGCGTTAGCTTCACACCTTCGGCAGCAGGAGGGCGGCAGCTTC |
| | TCGCAGGCGGCAGGGCGGGCGGCCAGGATCATGTCCACCACCACA |
| | TGCCAAGTGGTGGCGTTCCTCCTGTCCATCCTGGGGCTGGCCGGC |
| | TGCATCGCGGCCACCGGGATGGACATGTGGAGCACCCAGGACCTG |
| | TACGACAACCCCGTCACCTCCGTGTTCCAGTACGAAGGGCTCTGG |
| | AGGAGCTGCGTGAGGCAGAGTTCAGGCTTCACCGAATGCAGGCCC |
| | TATTTCACCATCCTGGGACTTCCAGCCATGCTGCAGGCAGTGCGA |
| | GCCCTGATGATCGTAGGCATCGTCCTGGGTGCCATTGGCCTCCTG |
| | GTATCCATCTTTGCCCTGAAATGCATCCGCATTGGCAGCATGGAG |
| | GACTCTGCCAAAGCCAACATGACACTGACCTCCGGGATCATGTTC |
| | ATTGTCTCAGGTCTTTGTGCAATTGCTGGAGTGTCTGTGTTTGCC |
| | AACATGCTGGTGACTAACTTCTGGATGTCCACAGCTAACATGTAC |
| | ACCGGCATGGGTGGGATGGTGCAGACTGTTCAGACCAGGTACACA |
| | TTTGGTGCGGCTCTGTTCGTGGGCTGGGTCGCTGGAGGCCTCACA |
| | CTAATTGGGGGTGTGATGATGTGCATCGCCTGCCGGGGCCTGGCA |
| | CCAGAAGAAACCAACTACAAAGCCGTTTCTTATCATGCCTCAGGC |
| | CACAGTGTTGCCTACAAGCCTGGAGGCTTCAAGGCCAGCACTGGC |
| | TTTGGGTCCAACACCAAAAACAAGAAGATATACGATGGAGGTGCC |
| | CGCACAGAGGACGAGGTACAATCTTATCCTTCCAAGCACGACTAT |
| | GTGTAATGCTCTAAGACCTCTCAGCACGGGCGGAAGAAACTCCCG |
| | GAGAGCTCACCCAAAAAACAAGGAGATCCCATCTAGATTTCTTCT |
| | TGCTTTTGACTCACAGCTGGAAGTTAGAAAAGCCTCGATTTCATC |
| | TTTGGAGAGGCCAAATGGTCTTAGCCTCAGTCTCTGTCTCTAAAT |
| | ATTCCACCATAAAACAGCTGAGTTATTTATGAATTAGAGGCTATA |
| | GCTCACATTTTCAATCCTCTATTTCTTTTTTTAAATATAACTTTC |
| | TACTCTGATGAGAGAATGTGGTTTTAATCTCTCTCTCACATTTTG |
| | ATGATTTAGACAGACTCCCCCTCTTCCTCCTAGTCAATAAACCCA |
| | TTGATGATCTATTTCCCAGCTTATCCCCAAGAAAACTTTTGAAAG |
| | GAAAGAGTAGACCCAAAAATGTTATTTTCTGCTGTTTGAATTTTG |

| | |
|---|---|
| ORF Start: ATG at 301-3 | ORF Stop: TGA at 1085-7 |
| SEQ ID NO: 10 | 261 aa |

| | |
|---|---|
| NOV3a, | MetSerThrThrThrCysGlnValValAlaPheLeuLeuSerIle |
| NOV3 Protein | |
| Sequence | LeuGlyLeuAlaGlyCysIleAlaAlaThrGlyMetAspMetTrp |
| | SerThrGlnAspLeuTyrAspAsnProValThrSerValPheGln |
| | TyrGluGlyLeuTrpArgSerCysValArgGlnSerSerGlyPhe |
| | ThrGluCysArgProTyrPheThrIleLeuGlyLeuProAlaMet |
| | LeuGlnAlaValArgAlaLeuMetIleValGlyIleValLeuGly |
| | AlaIleGlyLeuLeuValSerIlePheAlaLeuLysCysIleArg |
| | IleGlySerMetGluAspSerAlaLysAlaAsnMetThrLeuThr |
| | SerGlyIleMetPheIleValSerGlyLeuCysAlaIleAlaGly |
| | ValSerValPheAlaAsnMetLeuValThrAsnPheTrpMetSer |
| | ThrAlaAsnMetTyrThrGlyMetGlyGlyMetValGlnThrVal |
| | GlnThrArgTyrThrPheGlyAlaAlaLeuPheValGlyTrpVal |
| | AlaGlyGlyLeuThrLeuIleGlyGlyValMetMetCysIleAla |
| | CysArgGlyLeuAlaProGluGluThrAsnTyrLysAlaValSer |
| | TyrHisAlaSerGlyHisSerValAlaTyrLysProGlyGlyPhe |
| | LysAlaSerThrGlyPheGlySerAsnThrLysAsnLysLysIle |
| | TyrAspGlyGlyAlaArgThrGluAspGluValGlnSerTyrPro |
| | SerLysHisAspTyrVal |

| | |
|---|---|
| SEQ ID NO: 11 | 838 bp |

| | |
|---|---|
| NOV3b, | TGGCGGCAGGGCGGGCGGCCAGGATCATGTCCACCACCACATGCCAAGTGGTGGCGTTCC |
| CG52234-02 | |
| DNA Sequence | TCCTGTCCATCCTGGGGCTGGCCGGCTGCATCGCGGCCACCGGGATGGACATGTGGAGCA |
| | CCCAGGACCTGTACGACAACCCCGTCACCTCCGTGTTCCAGTACGAAGGGCTCTGGAGGA |
| | GCTGCGTGAGGCAGAGTTCAGGCTTCACCGAATGCAGGCCCTATTTCACCATCCTGGGAC |
| | TTCCAGCCATGCTGCAGGCAGTGCGAGCCCTGATGATCGTAGGCATCGTCCTGGGTGCCA |
| | TTGGCCTCCTGGTATCCATCTTTGCCCTGAAATGCATCCGCATTGGCAGCATGGAGGACT |
| | CTGCCAAAGCCAACATGACACTGACCTCCGGGATCATGTTCATTGTCTCAGGTCTTTGTG |
| | CAATTGCTGGAGTGTCTGTGTTTGCCAACATGCTGGTGACTAACTTCTGGATGTCCACAG |
| | CTAACATGTACACCGGCATGGGTGGGATGGTGCAGACTGTTCAGACCAGGTACACATTTG |
| | GTGCGGCTCTGTTCGTGGGCTGGGTCGCTGGAGGCCTCACACTAATTGGGGGTGTGATGA |
| | TGTGCATCGCCTGCCGGGGCCTGGCACCAGAAGAAACCAACTACAAAGCCGTTTCTTATC |
| | ATGCCTCAGGCCACAGTGTTGCCTACAAGCCTGGAGGCTTCAAGGCCAGCACTGGCTTTG |
| | GGTCCAACACCAAAAACAAGAAGATATACGATGGAGGTGCCCGCACAGAGGACGAGGTAC |
| | AATCTTATCCTTCCAAGCACGACTATGTGTAATGCTCTAAGACCTCTCAGCACGGGCA |

| | |
|---|---|
| ORF Start: ATG at 27-29 | ORF Stop: TAA at 810-812 |

| | | |
|---|---|---|
| SEQ ID NO: 12 | 261 aa | MW at kD |

| | |
|---|---|
| NOV3b, | MSTTTCQVVAFLLSILGLAGCIAATGMDMWSTQDLYDNPVTSVFQYEGLWRSCVRQSSGF |
| CG52234-02 | |
| Protein Sequence | TECRPYFTILGLPAMLQAVRALMIVGIVLGAIGLLVSIFALKCIRIGSMEDSAKANMTLT |
| | SGIMFIVSGLCAIAGVSVFANMLVTNFWMSTANMYTGMGGMVQTVQTRYTFGAALFVGWV |
| | AGGLTLIGGVMMCIACRGLAPEETNYKAVSYHASGHSVAYKPGGFKASTGFGSNTKNKKI |
| | YDGGARTEDEVQSYPSKHDYV |

| | |
|---|---|
| SEQ ID NO: 13 | 1530 bp |

| | |
|---|---|
| Nov3b, reverse | caaaattcaaacagcagaaaataacatttttgggtctactctttcctttcaaaagttttc |
| NOV3 DNA | |
| Sequence | ttggggataagctgggaaatagatcatcaatgggtttattgactaggaggaagaggggga |
| | gtctgtctaaatcatcaaaatgtgagagagagattaaaaccacattctctcatcagagta |
| | gaaagttatatttaaaaaaagaaatagaggattgaaaatgtgagctatagcctctaattc |
| | ataaataactcagctgttttatggtggaatatttagagacagagactgaggctaagacca |
| | tttggcctctccaaagatgaaatcgaggcttttctaacttccagctgtgagtcaaaagca |
| | agaagaaatctagatgggatctccttgttttttgggtgagctctccgggagtttcttccg |
| | cccgtgctgagaggtcttagagcattacacatagtcgtgcttggaaggataagattgtac |
| | ctcgtcctctgtgcgggcacctccatcgtatatcttcttgtttttggtgttggacccaaa |
| | gccagtgctggccttgaagcctccaggcttgtaggcaacactgtggcctgaggcatgata |
| | agaaacggctttgtagttggtttcttctggtgccaggccccggcaggcgatgcacatcat |
| | cacacccccaattagtgtgaggcctccagcgacccagcccacgaacagagccgcaccaaa |
| | tgtgtacctggtctgaacagtctgcaccatcccacccatgccggtgtacatgttagctgt |
| | ggacatccagaagttagtcaccagcatgttggcaaacacagacactccagcaattgcaca |
| | aagacctgagacaatgaacatgatcccggaggtcagtgtcatgttggctttggcagagtc |
| | ctccatgctgccaatgcggatgcatttcagggcaaagatggataccaggaggccaatggc |
| | acccaggacgatgcctacgatcatcagggctcgcactgcctgcagcatggctggaagtcc |
| | caggatggtgaaatagggcctgcattcggtgaagcctgaactctgcctcacgcagctcct |
| | ccagagcccttcgtactggaacacggaggtgacggggttgtcgtacaggtcctgggtgct |
| | ccacatgtccatcccggtggccgcgatgcagccggccagccccaggatggacaggaggaa |
| | cgccaccacttggcatgtggtggtggacatgatcctggccgcccgccctgccgcctgcga |
| | gaagctgccgccctcctgctgccgaaggtgtgaagctaacgcgctcctgaggggagctct |
| | tctaatcagatggtttccctaggctctcagtatttgctcacagtgttttccttagatagc |
| | tcagtttcgctcctggttcaagggcattatttttacatttgttagagaagcgggcccaag |
| | aggcacttgtctgctctccgcaggccgggtgggttcagcgcagagccgtgccagggagca |
| | gaaggactccctgatccgggccagaccggc |

Sequence comparison of the above protein sequences yields the following sequence relationships shown in Table 3B.

TABLE 3B
Comparison of NOV3a against NOV3b.

| | NOV3a | Identities/ |
|---|---|---|
| Protein | Residues/ | Similarities for |
| Sequence | Match Residues | the Matched Region |
| NOV3b | 1 . . . 838 | 836/838 (99%) |

Further analysis of the NOV1c protein yielded the following properties shown in Table 3C.

TABLE 3C
Protein Sequence Properties NOV3a

| | |
|---|---|
| PSort | The protein is most likely localized to the plasma membrane. |
| analysis: | plasma membrane—Certainty = 0.6400 |
| | Golgi body—Certainty = 0.4600 |
| | endoplasmic reticulum (membrane)—Certainty = 0.3700 |
| | endoplasmic reticulum (lumen)—Certainty = 0.1000 |
| | INTEGRAL Likelihood = −10.93 Transmembrane 82-98 |
| | (68-109) |
| | INTEGRAL Likelihood = −7.54 Transmembrane 123-139 |
| | (117-147) |
| | INTEGRAL Likelihood = −4.35 Transmembrane 180-196 |
| | (170-196) |
| | Likely a Type IIIa membrane protein (clv) |
| SignalP | Most likely cleavage site between pos. 23 and 24: CIA-AT |
| analysis: | |

The NOV3 nucleic acid sequence was searched against the GenBank database using BLASTP search protocols. A similarity of 56% (110 of 196 amino acids) was found to human claudin-1 (GenBank Accession Number TREMBLNEW:AAD22062), which is a protein of 211 amino acids. Proteins of the claudin family are integral membrane proteins with four transmembrane domains and are found in tight junctions (Furuse et al., 1998, J. Cell Biol. 141:1539-50). NOV3 proteins and claudin proteins may thus share at least some activities. Claudins are a newly discovered family of integral membrane proteins implicated in maintenance of the intercellular tight junctions (Morita et al., PNAS 96: 511-16 (1999). They occur at the most apical portion of polarized epithelial and endothelial cells, and serve to prevent intercellular transport of solutes. They occur in many tissues, including liver and kidney. Accordingly, the claudin-like protein of the present invention or its gene will be useful in therapeutic intervention in human subjects in whom this gene or the product protein is a defective allele.

In a further BLAST search of public sequence databases, the NOV3a protein was found to have homology to the proteins shown in the BLASTP data in Table 3D.

TABLE 3D
Public BLASTP Results for NOV3a

| | | NOV3a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| P56856 | Claudin-18— | 1 . . . 261 | 261/261 (100%) | 1.8e−138 |
| | Human, 261 aa | 1 . . . 261 | | |

PFam domain analysis for the NOV1a protein is provided in Table 3E.

TABLE 3E
Domain Analysis of NOV3a

| | | Identities/ | |
|---|---|---|---|
| | | Similarities for | Expect |
| Pfam Domain | NOV3a Match Region | the Matched Region | Value |
| PMP22_Claudin: | domain 1 of 1; EMP/ | nt 278 . . . 469 | 1.5e−30 |
| | MP20 family | | |

This indicates that the sequence of the invention has properties similar to those of other proteins known to contain this/these domain(s) and similar to the properties of these domains. Further analysis of NOV3 is shown in the Examples.

NOV4

The NOV4 clones (also referred to as Clone NOV4 or CG54942-01) were analyzed, and the nucleotide and predicted polypeptide sequences are shown in Table 4A.

TABLE 4A
NOV4 Sequence Analysis

| | |
|---|---|
| SEQ ID NO: 14 | 603 bp |

| | | |
|---|---|---|
| NOV4a, | 1 | CACAGAGCCTGGGCTGCAGGCACCTCCCTGCCAGCTCTCCCGCTC |
| NOV4 DNA Sequence | | |
| | 46 | CTGGCACCGCCGCCCGACCTGCCTTCTGAGCCCGGTGAACTGCGC |
| | 91 | CGCGCCCCCCGCTGTCCCCCGCGCTCCCCGGCTACTGCGGGCGGC |
| | 136 | GCTGCTGCTCCTGCTCCTGGTAGCCTCCGGCCGGCGAGCGGCAGG |
| | 181 | AGTATGGGTGGCCCATGAACTGCCTTGCCAGTGCTTGCAGACCCT |
| | 226 | GCAGGGAATTCACCCCAAGAATATCCGAAGTGTGAACGTGAAGTC |
| | 271 | CCCTGGACCCCACTGCACCCAAACCGAAGTCATATAAGTCCCTCC |
| | 316 | CCGTGACTTTTTCTTTTCTCAGACCATGAGAATTAAATCTGTAGT |
| | 361 | CATTTTTCTAATTAGTGGCTGGATCCAAAAGAATAATAAAATATA |
| | 406 | TCTAATCTCCCCGAAGAAAGCCCAAAGGTTACATCCAGGACTTGG |
| | 451 | TCCTAGGTTAAGCCCTAAGGTGCTGGGGAGAGTGGAATGCTATCT |
| | 496 | TCCTAATTATTTACATATCAAAAGAGATGAAGCCCACAGAACCTA |
| | 541 | AAGACATCAGTAGGACACATAAATTGAAGACCAGAGGGCTCTTAG |
| | 596 | GTTCCAGGGGAAAGGTAT |

| | |
|---|---|
| ORF Start: ATG at 341 | ORF Stop: TAA at 539 |

| | | |
|---|---|---|
| SEQ ID NO: 15 | 66 aa | MW at kD |

| | |
|---|---|
| NOV4a, | MetArgIleLysSerValValIlePheLeuIleSerGlyTrpIle |
| NOV4 Protein Sequence | |
| | GlnLysAsnAsnLysIleTyrLeuIleSerProLysLysAlaGln |
| | ArgLeuHisProGlyLeuGlyProArgLeuSerProLysValLeu |
| | GlyArgValGluCysTyrLeuProAsnTyrLeuHisIleLysArg |
| | AspGluAlaHisArgThr |

| | |
|---|---|
| SEQ ID NO: 16 | 207 bp |

| | |
|---|---|
| NOV4b, | GGAGTATGGGTGGCCCATGAACTGCCTTGCCAGTGCTTGCAGACCCTGCAGGGAATTCAC |
| CG54942-01 DNA | |
| Sequence | CCCAAGAATATCCGAAGTGTGAACGTGAAGTCCCGTGGACCCCCCTGCACCCAAACGGAA |
| | GTCATAGCCACACTCAAGAATGGGAAGAAAGCTTGTCTCAACCCCGCATCCCCCATGGTT |
| | CAGAAAATCATCGAAAAGATACTGAAC |

| | |
|---|---|
| ORF Start: GGA at 1 | ORF Stop: AAC at 207 |
| SEQ ID NO: 17 | |

| | |
|---|---|
| NOV4b, | GVWVAHELPCQCLQTLQGIHPKNIRSVNVKSRGPPCTQTEVIATL |
| CG54942-01 Protein | |
| Sequence | KNGKKACLNPASPMVQKIIEKILN |

Sequence comparison of the above protein sequences yields the following sequence relationships shown in Table 4B.

TABLE 4B
Comparison of NOV4a against NOV4b.

| | NOV4a | Identities/ |
|---|---|---|
| Nucleic | Residues/ | Similarities for |
| Acid Sequence | Match Residues | the Matched Region |
| NOV4a | 179 . . . 304 | 123/126 (98%) |
| NOV4b | 1 . . . 126 | |

TABLE 4C
Protein Sequence Properties NOV4a

| | |
|---|---|
| PSort analysis: | Psort Results (see Details): |
| | 55.0%: endoplasmic reticulum (membrane) |
| | 31.2%: lysosome (lumen) |
| | 10.0%: endoplasmic reticulum (lumen) |
| | 10.0%: outside |
| | Psort II Results (see Details): |
| | 60.9%: nuclear |
| | 34.8%: mitochondrial |
| | 4.3%: cytoplasmic |
| SignalP analysis: | cleavage site between position 17 and 18 (IQK-NN) |
| NNCN: | Reinhardt's method for Cytplasmic/Nuclear |
| | discrimination |
| | Prediction: nuclear |
| | Reliability: 76.7 |

The NOV4 nucleotide sequence was searched against the GenBank database using BLASTP search protocols. No significant homologies were found. A search against the GenBank database using BLASTN search proteins showed two regions with 100% identities to human MGSA/GRO pseudogene sequence (GenBank Accession Number U88432). One region with identity was found between nucleotides 1-179 of NOV4 and nucleotides 1148 and 1326 of MGSA-GRO pseudogene. The second region of identity was found between nucleotides 180-603 of NOV4 and nucleotides 1425 and 1848 of MGSA-GRO pseudogene (MGSA/GRO pseudogene sequence has an intron between nucleotides 1327 an 1424). The NOV4 sequence showed 82% identities (251 of 303 nucleotides) to human cytokine (GRO-beta) mRNA sequence (GenBank Accession Number M36820). See, e.g., Sager et al. 1990 Proc Natl Acad Sci U S A 87(19): 7732-6.

In a BLASTP search of public sequence databases, the NOV4a protein was found to have no homology to the known proteins disclosed in GenBank. PFam analysis of NOV4a protein for known domains predicted no hits above threshold.

FIZZ-X (FIZZ-3 LIKE)

The FIZZX clones (also referred to herein as clone FIZZX, FIZZXf, 246826889, 2468237306 and CG51604-02revcomp) were analyzed, and the nucleotide and predicted polypeptide sequences are shown in Table 5A.

TABLE 5A
FIZZX Sequence Analysis

| | |
|---|---|
| SEQ ID NO: 18 | 505 bp |

| | | |
|---|---|---|
| FIZZXa, | 1 | AAAGAAAGAGCTGCGGTGCAGGAATTCGTGTGCCGGATTTGGTTA |
| FIZZXf DNA | | |
| Sequence | 46 | GCTGAGCCCACCGAGAGGCGCCTGCAGAATGAAAGCTCTCTGTCT |
| | 91 | CCTCCTCCTCCCTGTCCTGGGGCTGTTGGTGTCTAGCAAGACCCT |
| | 136 | GTGCTCCATGGAAGAAGCCATCAATGAGAGGATCCAGGAGGTCGC |
| | 181 | CGGCTCCCTAATATTTAGGGCAATAAGCAGCATTGGCCTGGAGTG |
| | 226 | CCAGAGCGTCACCTCCAGGGGGGACCTGGCTACTTGCCCCCGAGG |
| | 271 | CTTCGCCGTCACCGGCTGCACTTGTGGCTCCGCCTGTGGCTCGTG |
| | 316 | GGATGTGCGCGCCGAGACCACATGTCACTGCCAGTGCGCGGGCAT |
| | 361 | GGACTGGACCGGAGCGCGCTGCTGTCGTGTGCAGCCCTGAGGTCG |
| | 406 | CGCGCAGCCCCACAGTGGACGCGGGCGGAAGGCGGCTCCAGGTCC |
| | 451 | GGAGGGGTTGCGGGGGAGCTGGAAATAAACCTGGAGATGATGATG |
| | 496 | ATGATGATGA |

| | |
|---|---|
| ORF Start: ATG at 148 | ORF Stop: TGA at 403 |

| | | |
|---|---|---|
| SEQ ID NO: 19 | 108 aa | MW at 11419.2 kD |

| | |
|---|---|
| FIZZXa, | MetLysAlaLeuCysLeuLeuLeuLeuProValLeuGlyLeuLeu |
| FIZZXf Protein | |
| Sequence | ValSerSerLysThrLeuCysSerMetGluGluAlaIleAsnGlu |
| | ArgIleGlnGluValAlaGlySerLeuIlePheArgAlaIleSer |
| | SerIleGlyLeuGluCysGlnSerValThrSerArgGlyAspLeu |
| | AlaThrCysProArgGlyPheAlaValThrGlyCysThrCysGly |
| | SerAlaCysGlySerTrpAspValArgAlaGluThrThrCysHis |
| | CysGlnCysAlaGlyMetAspTrpThrGlyAlaArgCysCysArg |
| | ValGlnPro |

| | |
|---|---|
| SEQ ID NO: 20 | 487 bp |

| | |
|---|---|
| FIZZXb, | AATTCGTGTGCCGGATTTGGTTAGCTGAGCCCACCGAGAGGCGCCTGCAG |
| CG51604- | |
| 02_revcomp | GATGAAAGCTCTCTGTCTCCTCCTCCTCCCTGTCCTGGGGCTGTTGGTGT |
| DNA Sequence | |
| | CTAGCAAGACCCTGTGCTCCATGGAAGAAGCCATCAATGAGAGGATCCAG |
| | GAGGTCGCCGGCTCCCTAATATTTAGGGCAATAAGCAGCATTGGCCTGGA |
| | GTGCCAGAGCGTCACCTCCAGGGGGGACCTGGCTACTTGCCCCCGAGGCT |
| | TCGCCGTCACCGGCTGCACTTGTGGCTCCGCCTGTGGCTCGTGGGATGTG |
| | CGCGCCGAGACCACATGTCACTGCCAGTGCGCAGGCATGGACTGGACCGG |
| | AGCGCGCTGCTGTCGTGTGCAGCCCTGAGGTCGCGCGCAGCGCGTGCACA |
| | GCGCGGGCGGAGGCGGCTCCAGGTCCGGAGGGGTTGCGGGGGAGCTGGAA |
| | ATAAACCTGGAGATGATGATGATGATGATGATGGGGT |

| | |
|---|---|
| ORF Start: ATG at 52 | ORF Stop: TGA at 376 |
| SEQ ID NO: 21 | 108 aa |

| | |
|---|---|
| FIZZXb, | MKALCLLLLPVLGLLVSSKTLCSMEEAINERIQEVAGSLIFRAISSIGLE |
| CG51604- | |
| 02_revcomp | CQSVTSRGDLATCPRGFAVTGCTCGSACGSWDVRAETTCHCQCAGMDWTG |
| Protein | |
| Sequence | ARCCRVQP |

| | |
|---|---|
| SEQ ID NO: 22 | 348 bp |

| | |
|---|---|
| FIZZXc, | CACCAGATCTCCACCATGAAAGCTCTCTGTCTCCTCCTCCTCCCTGTCCTGGGGCT |
| 246836889 | |
| DNA Sequence | GTTGGTGTCTAGCAAGACCCTGTGCTCCATGGAAGAAGCCATCAATGAGAGGATCC |
| | AGGAGGTCGCCGGCTCCCTAATATTTAGGGCAATAAGCAGCATTGGCCTGGAGTGC |
| | CAGAGCGTCACCTCCAGGGGGGACCTGGCTACTTGCCCCCGAGGCTTCGCCGTCAC |
| | CGGCTGCACTTGTGGCTCCGCCTGTGGCTCGTGGGATGTGCGCGCCGAGACCACAT |
| | GTCACTGCCAGTGCGCAGGCATGGACTGGACCGGAGCGCGCTGCTGTCGTGTGCAC |
| | CCCGTCGACGGC |

| | |
|---|---|
| ORF Start: CAC at 1 | ORF Stop: GGC at 346 |
| SEQ ID NO: 23 | 116 aa |

| | |
|---|---|
| FIZZXc, | HQISTMKALCLLLLPVLGLLVSSKTLCSMEEAINERIQEVAGSLIFRAIS |
| 246826889 | |
| Protein | SIGLECQSVTSRGDLATCPRGFAVTGCTCGSACGSWDVRAETTCHCQCAG |
| Sequence | |
| | MDWTGARCCRVQPVDG |

| | |
|---|---|
| SEQ ID NO: 24 | 304 bp |

| | |
|---|---|
| FIZZXd, | CACCAGATCTCTGTTGGTGTCTAGCAAGACCCTGTGCTCCATGGAAGAAG |
| mature | |
| 2468237306 | CCATCAATGAGAGGATCCAGGAGGTCGCCGGCTCCCTAATATTTAGGGCA |
| DNA Sequence | |
| | ATAAGCAGCATTGGCCTGGAGTGCCAGAGCGTCACCTCCAGGGGGGACCT |
| | GGCTACTTGCCCCCGAGGCTTCGCCGTCACCGGCTGCACTTGTGGCTCCG |
| | CCTGTGGCTCGTGGGATGTGCGCGCCGAGACCACATGTCACTGCCAGTGC |
| | GCAGGCATGGACTGGACCGGAGCGCGCTGCTGTCGTGTGCAGCCCGTCGA |
| | CGGC |

| | |
|---|---|
| ORF Start: ACC at 2 | ORF Stop: GGC at 302 |
| SEQ ID NO: 25 | 101 aa |

| | |
|---|---|
| FIZZXd, | TRSLLVSSKTLCSMEEAINERIQEVAGSLIFRAISSIGLECQSVTSRGDL |
| mature | |
| 2468237306 | ATCPRGFAVTGCTCGSACGSWDVRAETTCHCQCAGMDWTGARCCRVQPVD |
| Protein | |
| Sequence | G |

Additional variant FIZZX sequences are contemplated that have modified 5′ and 3′ nucleic acid sequences, such as cDNA sequences containing the complete open reading frame and flanked by restriction enzymes, linkers, or cloning vector sequence. In a certain embodiment, the disclosed nucleic acid of FIZZXa can have the residues “TGG” extending on the 3′ end. In an alternative embodiment, the FIZZXa nucleic acid can have the residues “TGCCCTT” as a 5′ extention to the disclosed sequence in Table 5A.

Sequence comparison of the above protein sequences yields the following sequence relationships shown in Table 5B.

[image]

[image]

Further analysis of the FIZZXa protein yielded the following properties shown in Table 5C.

TABLE 5C
Protein Sequence Properties FIZZXa

| | |
|---|---|
| PSort | most likely located outside of the cell |
| analysis: | outside—Certainty = 0.7857 |
| | endoplasmic reticulum (membrane)—Certainty = 0.1000 |
| | endoplasmic reticulum (lumen)—Certainty = 0.1000 |
| | lysosome (lumen)—Certainty = 0.1000 |
| SignalP | Most likely cleavage site between pos. 22 and 23: TLC-SM |
| analysis: | |

A BLASTN search of the FIZZXa protein against the Geneseq database, a proprietary database that contains sequences published in patents and patent publication, yielded several homologous proteins shown in Table 5D. Further search results are shown in Table 5D.

TABLE 5D
BLASTN Results for FIZZXa

| | Protein/ | | | |
|---|---|---|---|---|
| | Organism/ | FIZZXa | Identities/ | |
| | Length | Residues/ | Similarities | |
| Geneseq | [Patent #, | Match | for the | Expect |
| Identifier | Date] | Residues | Matched Region | Value |
| GENBANK— | Homo | 57 . . . 513 | 457/457 (100%) | 7.3e−97 |
| TD:AF205952 | sapiens | 1 . . . 457 | 457/457 (100%) | |
| | cysteine-rich | | | |
| | secreted | | | |
| | protein | | | |
| | (FIZZ3) | | | |
| | mRNA, | | | |
| | complete | | | |
| | cds—Homo | | | |
| | sapiens, | | | |
| | 457 bp. | | | |

In a search of public databases, FIZZXa was found to be homologous to the novel 9.4 kDa cysteine-rich secreted protein, FIZZ1 (found in inflammatory zone). Murine (m) FIZZ1 is the founding member of a new gene family including two other murine genes expressed, respectively, in intestinal crypt epithelium and white adipose tissue, and two related human genes. See, PubMed ID: 10921885. During allergic pulmonary inflammation, mFIZZ1 expression markedly increases in hypertrophic, hyperplastic bronchial epithelium and appears in type II alveolar pneumocytes. In vitro, recombinant mFIZZ1 inhibits the nerve growth factor (NGF)-mediated survival of rat embryonic day 14 dorsal root ganglion (DRG) neurons and NGF-induced CGRP gene expression in adult rat DRG neurons. In vivo, FIZZ1 may modulate the function of neurons innervating the bronchial tree, thereby altering the local tissue response to allergic pulmonary inflammation. See, PubMed ID: 10921885.

In a search of proprietary databases, the FIZZXa amino acid sequence of 108 amino acids was found to be 100% identical to the sequence of a cysteine rich soluble protein designated C23 (Accession Number W87710, patent application Ser. No. WO98/58061, Schering Corp.) and to a human secreted polypeptide (Accession Number Y12933, Patent application WO99/11293, Human Genome Sciences, Inc). Further searches using the GenBank database BLASTP showed some homology to rat MEGF6 protein (SPTREMBL-ACC:O88281).

In a further BLASTP search of public sequence databases, the FIZZXa protein was found to have homology to the proteins shown in the BLASTP data in Table 5E.

TABLE 5E
Public BLASTP Results for FIZZXa

| | | FIZZXa | Identities/ | |
|---|---|---|---|---|
| Protein | Protein/ | Residues/ | Similarities | |
| Accession | Organism/ | Match | for the | Expect |
| Number | Length | Residues | Matched Region | Value |
| TREMBLNEW— | (CYS- | 1 . . . 108 | 108/108 (100%) | 3.3e−56 |
| ACC:AAG02144 | TEINE- | 1 . . . 108 | 108/108 (100%) | |
| | RICH | | | |
| | SECRETED | | | |
| | PROTEIN | | | |
| | Homo | | | |
| | sapiens | | | |
| | (Human), | | | |
| | 108 aa. | | | |
| AAG59823 | RESISTIN— | 114 | NA | NA |
| | Mus | | | |
| | musculus | | | |
| | (Mouse) | | | |
| AA059827 | RESISTIN- | 111 | NA | NA |
| | LIKE MOL- | | | |
| | ECULE | | | |
| | BETA | | | |

PFam analysis found no significant homologies to known domains in the FIZZXa protein.

The nucleic acids and proteins of the invention have applications in the diagnosis and/or treatment of various diseases and disorders. For example, the compositions of the present invention will have efficacy for the treatment of patients suffering from: autoimmune disease, allergies, immunodeficiencies, asthma, emphysema, hemophilia, hypercoagulation, idiopathic thrombocytopenic purpura, transplantation, graft versus host disease, systemic lupus erythematosus, scleroderma, ARDS, anemia, ataxia-telangiectasia, fertility, as well as other diseases, disorders and conditions.

The cDNA of clone FIZZX was inserted into expression vectors for mammalian embryonic kidney 293 and insect (baculo) cell expression. The protein expressed in mammalian cells was secreted as 20 kDa protein. The protein secreted by Sf9 insect cells is about 45 kDa. Further analysis of FIZZX is provided in the Examples.

MAMM-X (MAMMAGLOBIN)

TABLE 6A
MAMMX Sequence Analysis

| | |
|---|---|
| SEQ ID NO: 26 | 517 bp |

| | | |
|---|---|---|
| MAMMX, | 1 | CCTCCACAGCAACTTCCTTGATCCCTGCCACGCACGACTGAACAC |
| 2353875f DNA Sequence | | |
| | 46 | AGACAGCAGCCGCCTCGCCATGAAGCTGCTGATGGTCCTCATGCT |
| | 91 | GGCGGCCCTCCTCCTGCACTGCTATGCAGATTCTGGCTGCAAACT |
| | 136 | CCTGGAGGACATGGTTGAAAAGACCATCAATTCCGACATATCTAT |
| | 181 | ACCTGAATACAAAGAGCTTCTTCAAGAGTTCATAGACAGTGATGC |
| | 226 | CGCTGCAGAGGCTATGGGGAAATTCAAGCAGTGTTTCCTCAACCA |
| | 271 | GTCACATAGAACTCTGAAAAACTTTGGACTGATGATGCATACAGT |
| | 316 | GTACGACAGCATTTGGTGTAATATGAAGAGTAATTAACTTTACCC |
| | 361 | AAGGCGTTTGGCTCAGAGGGCTACAGACTATGGCCAGAACTCATC |
| | 406 | TGTTGATTGCTAGAAACCACTTTTCTTTCTTGTGTTGTCTTTTTA |
| | 451 | TGTGGAAACTGCTAGACAACTGTTGAAACCTCAAATTCATTTCCA |
| | 496 | TTTCAATAACTAACTGCAAATC |

| | |
|---|---|
| ORF Start: ATG at 65 | ORF Stop: TAA at 350 |

| | | |
|---|---|---|
| SEQ ID NO: 27 | 95 aa | MW at kD |

| | |
|---|---|
| MAMMX, | MetLysLeuLeuMetValLeuMetLeuAlaAlaLeuLeuLeuHis |
| 2353875f Protein Sequence | |
| | CysTyrAlaAspSerGlyCysLysLeuLeuGluAspMetValGlu |
| | LysThrIleAsnSerAspIleSerIleProGluTyrLysGluLeu |
| | LeuGlnGluPheIleAspSerAspAlaAlaAlaGluAlaMetGly |
| | LysPheLysGlnCysPheLeuAsnGlnSerHisArgThrLeuLys |
| | AsnPheGlyLeuMetMetHisThrValTyrAspSerIleTrpCys |
| | AsnMetLysSerAsn |

The nucleotide sequence is 100% identical (517 of 517 nucleotides) to the mRNA sequence of human Mammaglobin B precursor (GenBank Accession Number AF071219; Becker et al., 1998, Genomics 54:70-78). The amino acid sequence is 100% identical to the sequence of a human mammaglobin homologue (Accession Number Y02590, patent application Ser. No. WO99/19487, Incyte Pharmaceuticals) and to human endometrial specific steroid-binding factor III (Accession Number W35804, patent application Ser. No. WO97/34997, Human Genome Sciences, Inc.). Mammaglobin is a potential marker of breast cancer nodal metastasis and is expressed in primary, metastatic and occult breast cancer cells. Based upon homology, Mamm-X proteins and each homologous protein or peptide may share at least some activity.

The cDNA of clone Mamm-X was inserted into expression vectors for mammalian embryonic kidney 293 cells and expressed as a 10 kDa protein in the cell pellet. No secreted form of Mamm-X was detected. Further analysis of the MammX clone is presented in the Examples.

Herein is described are nucleic acids, polypeptides, antibodies, therapeutics, and methods of using the afore-mentioned nucleic acids and their encoded polypeptides.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

In one specific embodiment, the invention includes an isolated polypeptide comprising an amino acid sequence selected from the group consisting of: (a) a mature form of the amino acid sequence selected from the group consisting of SEQ ID NOs: 2, 4, 6, 8, 10, 12, 15 and 17, or 19, 21, 23, 25 and 27; (b) a variant of a mature form of the amino acid sequence selected from the group consisting of SEQ ID NO: 2n, wherein n is an integer between 1 and 45, wherein any amino acid in the mature form is changed to a different amino acid, provided that no more than 15% of the amino acid residues in the sequence of the mature form are so changed; (c) an amino acid sequence selected from the group consisting of SEQ ID NOs: 2, 4, 6, 8, 10, 12, 15 and 17, or 19, 21, 23, 25 and 27; (d) a variant of the amino acid sequence selected from the group consisting of SEQ ID NOs: 2, 4, 6, 8, 10, 12, 15 and 17, or 19, 21, 23, 25 and 27, wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NOs: 2, 4, 6, 8, 10, 12, 15 and 17, or 19, 21, 23, 25 and 27; (b) a variant of a mature form of the amino acid sequence selected from the group consisting of SEQ ID NOs: 2, 4, 6, 8, 10, 12, 15 and 17, or 19, 21, 23, 25 and 27, wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NOs: 2, 4, 6, 8, 10, 12, 15 and 17, or 19, 21, 23, 25 and 27; (d) a variant of the amino acid sequence selected from the group consisting of SEQ ID NOs: 2, 4, 6, 8, 10, 12, 15 and 17, or 19, 21, 23, 25 and 27, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NOs: 2, 4, 6, 8, 10, 12, 15 and 17, or 19, 21, 23, 25 and 27 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 1, 3, 5, 7, 9, 11, 13, 14 and 16, or 18, 20, 22, 24 and 26; (b) a nucleotide sequence wherein one or more nucleotides in the nucleotide sequence selected from the group consisting of SEQ ID NO: 1, 3, 5, 7, 9, 11, 13, 14 and 16, or 18, 20, 22, 24 and 26 is changed from that selected from the group consisting of the chosen sequence to a different nucleotide provided that no more than 15% of the nucleotides are so changed; (c) a nucleic acid fragment of the sequence selected from the group consisting of SEQ ID NO: 1, 3, 5, 7, 9, 11, 13, 14 and 16, or 18, 20, 22, 24 and 26; and (d) a nucleic acid fragment wherein one or more nucleotides in the nucleotide sequence selected from the group consisting of SEQ ID NO: 1, 3, 5, 7, 9, 11, 13, 14 and 16, or 18, 20, 22, 24 and 26 is changed from that selected from the group consisting of the chosen sequence to a different nucleotide provided that no more than 15% of the nucleotides are so changed.

Nucleic Acids and Polypeptides

One aspect of the invention pertains to isolated nucleic acid molecules that encode NOV1, NOV2, NOV3, NOV4, FIZZX or MAMMX proteins or biologically active portions thereof, as well as nucleic acid fragments sufficient for use as hybridization probes to identify NOV1, NOV2, NOV3, NOV4, FIZZX or MAMMX-encoding nucleic acids (e.g., NOV1, NOV2, NOV3, NOV4, FIZZX or MAMMX mRNA) and fragments for use as PCR primers for the amplification or mutation of NOV1, NOV2, NOV3, NOV4, FIZZX or MAMMX nucleic acid molecules. The invention also pertains to polynucleotides and encoded proteins that have amino acid and corresponding codon substitutions within certain defined limits, as disclosed herein.

As used herein, the term “nucleic acid molecule” and/or “polynucleotide” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide, precursor form, or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (host cell) in which the gene product arises. Examples of such processing steps leading to a “mature” form of a polypeptide or protein include the cleavage of the N-terminal methionine residue encoded by the initiation codon of an ORF or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), and 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated NOV1, NOV2, NOV3, NOV4, FIZZX or MAMMX nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO: 1, 3, 5, 7, 9, 11, 13, 14 or 16, or 18, 20, 22, 24 or 26, or a complement of any of these nucleotide sequences, can be isolated using standard molecular biology techniques and the sequence information provided herein. Using all or a portion of the nucleic acid sequences of SEQ ID NO: 1, 3, 5, 7, 9, 11, 13, 14 or 16, or 18, 20, 22, 24 or 26 as a hybridization probe, NOV1, NOV2, NOV3, NOV4, FIZZX or MAMMX molecules can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOV1, NOV2, NOV3, NOV4, FIZZX or MAMMX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS: 2n-1, wherein n is an integer between 1 and 45, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO: 1, 3, 5, 7, 9, 11, 13, 14 or 16, or 18, 20, 22, 24 or 26. In another embodiment, an isolated nucleic acid molecule of the invention comprises a nucleic acid molecule that is a complement of the nucleotide sequence shown in SEQ ID NO: 1, 3, 5, 7, 9, 11, 13, 14 or 16, or 18, 20, 22, 24 or 26, or a portion of this nucleotide sequence. A nucleic acid molecule that is complementary to the nucleotide sequence shown in SEQ ID NO: 1, 3, 5, 7, 9, 11, 13, 14 or 16, or 18, 20, 22, 24 or 26 is one that is sufficiently complementary to the nucleotide sequence shown in SEQ ID NO: 1, 3, 5, 7, 9, 11, 13, 14 or 16, or 18, 20, 22, 24 or 26 that it can hydrogen bond with little or no mismatches to the nucleotide sequence shown in SEQ ID NO: 1, 3, 5, 7, 9, 11, 13, 14 or 16, or 18, 20, 22, 24 or 26, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO: 1, 3, 5, 7, 9, 11, 13, 14 or 16, or 18, 20, 22, 24 or 26, e.g., a fragment that can be used as a probe or primer or a fragment encoding a biologically active portion of NOV1, NOV2, NOV3, NOV4, FIZZX or MAMMX.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. In various embodiments, fragments may be at least about 6, 15, 30, 100, 250, 500, or 1000 amino acids in length. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 30%, 50%, 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for A NOVX polypeptide of species other than humans, including, but not limited to vertebrates, and thus can include, e.g., frog, mouse, rat, rabbit, dog, cat, cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding a human NOVX protein. Homologous nucleic acid sequences include those nucleic acid sequences that encode conservative amino acid substitutions (see below) in SEQ ID NOS: 2n-1, wherein n is an integer between 1 and 45, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequence determined from the cloning of the human NOV1, NOV2, NOV3, NOV4, FIZZX or MAMMX gene allows for the generation of probes and primers designed for use in identifying and/or cloning NOV1, NOV2, NOV3, NOV4, FIZZX or MAMMX homologues in other cell types, e.g. from other tissues, as well as NOV1, NOV2, NOV3, NOV4, FIZZX or MAMMX homologues from other mammals. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO: 1, 3, 5, 7, 9, 11, 13, 14 or 16, or 18, 20, 22, 24 or 26 or an anti-sense strand nucleotide sequence of SEQ ID NO: 1, 3, 5, 7, 9, 11, 13, 14 or 16, or 18, 20, 22, 24 or 26 or of a naturally occurring mutant of SEQ ID NO: 1, 3, 5, 7, 9, 11, 13, 14 or 16, or 18, 20, 22, 24 or 26.

Probes based on the human NOV1, NOV2, NOV3, NOV4, FIZZX or MAMMX nucleotide sequence can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissue which misexpress a NOV1, NOV2, NOV3, NOV4, FIZZX or MAMMX protein, such as by measuring a level of a NOV1, NOV2, NOV3, NOV4, FIZZX or MAMMX -encoding nucleic acid in a sample of cells from a subject e.g., detecting NOV1, NOV2, NOV3, NOV4, FIZZX or MAMMX mRNA levels or determining whether a genomic NOV1, NOV2, NOV3, NOV4, FIZZX or MAMMX gene has been mutated or deleted. “A polypeptide having a biologically active portion of NOV1, NOV2, NOV3, NOV4, FIZZX or MAMMX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of NOV1, NOV2, NOV3, NOV4, FIZZX or MAMMX” can be prepared by isolating a portion of SEQ ID NO: 1, 3, 5, 7, 9, 11, 13, 14 or 16, or 18, 20, 22, 24 or 26, that encodes a polypeptide having a NOV1, NOV2, NOV3, NOV4, FIZZX or MAMMX biological activity (the biological activities of the NOV1, NOV2, NOV3, NOV4, FIZZX or MAMMX proteins are described below), expressing the encoded portion of NOV1, NOV2, NOV3, NOV4, FIZZX or MAMMX protein (e.g., by recombinant expression in vitro) and assessing the activity of the encoded portion of NOV1, NOV2, NOV3, NOV4, FIZZX or MAMMX. NOV1, NOV2, NOV3, NOV4, FIZZX or MAMMX variants

Nucleic Acid and Polypeptide Variants

The invention further encompasses nucleic acid molecules that differ from the nucleotide sequence shown in SEQ ID NO: 1, 3, 5, 7, 9, 11, 13, 14 or 16, or 18, 20, 22, 24 or 26 due to degeneracy of the genetic code and thus encode the same NOV1, NOV2, NOV3, NOV4, FIZZX or MAMMX protein as that encoded by the nucleotide sequence shown in SEQ ID NO: 1, 3, 5, 7, 9, 11, 13, 14 or 16, or 18, 20, 22, 24 or 26. In another embodiment, an isolated nucleic acid molecule of the invention has a nucleotide sequence encoding a protein having an amino acid sequence shown in SEQ ID NO: 1, 3, 5, 7, 9, 11, 13, 14 or 16, or 18, 20, 22, 24 or 26.

In addition to the human NOV1, NOV2, NOV3, NOV4, FIZZX or MAMMX nucleotide sequence shown in SEQ ID NO: 1, 3, 5, 7, 9, 11, 13, 14 or 16, or 18, 20, 22, 24 or 26 it will be appreciated by those skilled in the art that DNA sequence polymorphisms that lead to changes in the amino acid sequences of NOV1, NOV2, NOV3, NOV4, FIZZX or MAMMX may exist within a population (e.g., the human population). Such genetic polymorphism in the NOV1, NOV2, NOV3, NOV4, FIZZX or MAMMX gene may exist among individuals within a population due to natural allelic variation. As used herein, the terms “gene” and “recombinant gene” refer to nucleic acid molecules comprising an open reading frame encoding a NOV1, NOV2, NOV3, NOV4, FIZZX or MAMMX protein, preferably a mammalian NOV1, NOV2, NOV3, NOV4, FIZZX or MAMMX protein. Such natural allelic variations can typically result in 1-5% variance in the nucleotide sequence of the NOV1, NOV2, NOV3, NOV4, FIZZX or MAMMX gene. Any and all such nucleotide variations and resulting amino acid polymorphisms in NOV1, NOV2, NOV3, NOV4, FIZZX or MAMMX that are the result of natural allelic variation and that do not alter the functional activity of NOV1, NOV2, NOV3, NOV4, FIZZX or MAMMX are intended to be within the scope of the invention.

Moreover, nucleic acid molecules encoding NOV1, NOV2, NOV3, NOV4, FIZZX or MAMMX proteins from other species, and thus that have a nucleotide sequence that differs from the human sequence of SEQ ID NO: 1, 3, 5, 7, 9, 11, 13, 14 or 16, or 18, 20, 22, 24 or 26, are intended to be within the scope of the invention. Nucleic acid molecules corresponding to natural allelic variants and homologues of the NOV1, NOV2, NOV3, NOV4, FIZZX or MAMMX cDNAs of the invention can be isolated based on their homology to the human NOV1, NOV2, NOV3, NOV4, FIZZX or MAMMX nucleic acids disclosed herein using the human cDNAs, or a portion thereof, as a hybridization probe according to standard hybridization techniques under stringent hybridization conditions. For example, a soluble human NOV1, NOV2, NOV3, NOV4, FIZZX or MAMMX cDNA can be isolated based on its homology to human membrane-bound NOV1, NOV2, NOV3, NOV4, FIZZX or MAMMX. Likewise, a membrane-bound human NOV1, NOV2, NOV3, NOV4, FIZZX or MAMMX cDNA can be isolated based on its homology to soluble human NOV1, NOV2, NOV3, NOV4, FIZZX or MAMMX.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 1, 3, 5, 7, 9, 11, 13, 14 or 16, or 18, 20, 22, 24 or 26. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOV1, NOV2, NOV3, NOV4, FIZZX or MAMMX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequence of SEQ ID NO: 1, 3, 5, 7, 9, 11, 13, 14 or 16, or 18, 20, 22, 24 or 26 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 1, 3, 5, 7, 9, 11, 13, 14 or 16, or 18, 20, 22, 24 or 26 , or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO: 1, 3, 5, 7, 9, 11, 13, 14 or 16, or 18, 20, 22, 24 or 26, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0. 1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative mutations

In addition to naturally-occurring allelic variants of the NOV1, NOV2, NOV3, NOV4, FIZZX or MAMMX sequence that may exist in the population, the skilled artisan will further appreciate that changes can be introduced by mutation into the nucleotide sequence of SEQ ID NO: 1, 3, 5, 7, 9, 11, 13, 14 or 16, or 18, 20, 22, 24 or 26, thereby leading to changes in the amino acid sequence of the encoded NOV1, NOV2, NOV3, NOV4, FIZZX or MAMMX protein, without altering the functional ability of the NOV1, NOV2, NOV3, NOV4, FIZZX or MAMMX protein. For example, nucleotide substitutions leading to amino acid substitutions at “non-essential” amino acid residues can be made in the sequence of SEQ ID NO: 1, 3, 5, 7, 9, 11, 13, 14 or 16, or 18, 20, 22, 24 or 26. A “non-essential” amino acid residue is a residue that can be altered from the wild-type sequence of NOV1, NOV2, NOV3, NOV4, FIZZX or MAMMX without significantly altering the biological activity, whereas an “essential” amino acid residue is required for biological activity. For example, amino acid residues that are conserved among the NOV1, NOV2, NOV3, NOV4, FIZZX or MAMMX proteins of the present invention, are predicted to be particularly unamenable to alteration.

Another aspect of the invention pertains to nucleic acid molecules encoding NOV1, NOV2, NOV3, NOV4, FIZZX or MAMMX proteins that contain changes in amino acid residues that are not essential for activity. Such NOV1, NOV2, NOV3, NOV4, FIZZX or MAMMX proteins differ in amino acid sequence from SEQ ID NO: 2, 4, 6, 8, 10, 12, 15 or 17, yet retain biological activity. In one embodiment, the isolated nucleic acid molecule comprises a nucleotide sequence encoding a protein, wherein the protein comprises an amino acid sequence at least about 45% of whose residues are identical to the amino acid sequence of SEQ ID NO: 2, 4, 6, 8, 10, 12, 15 or 17, respectively. Preferably, the protein encoded by the nucleic acid molecule is at least about 60% identical to SEQ ID NO: 2, 4, 6, 8, 10, 12, 15 or 17, more preferably at least about 70% identical to SEQ ID NO: 2, 4, 6, 8, 10, 12, 15 or 17, still more preferably at least about 80% identical to SEQ ID NO: 2, 4, 6, 8, 10, 12, 15 or 17, even more preferably at least about 90% identical to SEQ ID NO: 2, 4, 6, 8, 10, 12, 15 or 17, and most preferably at least about 95% identical to SEQ ID NO: 2, 4, 6, 8, 10, 12, 15 or 17.

An isolated nucleic acid molecule encoding a NOV1, NOV2, NOV3, NOV4, FIZZX or MAMMX protein homologous to the protein of SEQ ID NO: 2, 4, 6, 8, 10, 12, 15 or 17 can be created by introducing one or more nucleotide substitutions, additions or deletions into the nucleotide sequence of SEQ ID NO: 1, 3, 5, 7, 9, 11, 13, 14 or 16, or 18, 20, 22, 24 or 26 such that one or more amino acid substitutions, additions or deletions are introduced into the encoded protein.

Mutations can be introduced into SEQ ID NO: 1, 3, 5, 7, 9, 11, 13, 14 or 16, or 18, 20, 22, 24 or 26 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in NOV1, NOV2, NOV3, NOV4, FIZZX or MAMMX is replaced with another amino acid residue from the same side chain family. Alternatively, in another embodiment, mutations can be introduced randomly along all or part of a NOV1, NOV2, NOV3, NOV4, FIZZX or MAMMX coding sequence, such as by saturation mutagenesis, and the resultant mutants can be screened for NOV1, NOV2, NOV3, NOV4, FIZZX or MAMMX biological activity to identify mutants that retain activity. Following mutagenesis of SEQ ID NO: 1, 3, 5, 7, 9, 11, 13, 14 or 16, or 18, 20, 22, 24 or 26 , the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOV1, NOV2, NOV3, NOV4, FIZZX or MAMMX protein can be assayed for (1) the ability to form protein:protein interactions with other NOV1, NOV2, NOV3, NOV4, FIZZX or MAMMX proteins, other cell-surface proteins, or biologically active portions thereof, (2) complex formation between a mutant NOV1, NOV2, NOV3, NOV4, FIZZX or MAMMX protein and a NOV1, NOV2, NOV3, NOV4, FIZZX or MAMMX ligand; (3) the ability of a mutant NOV1, NOV2, NOV3, NOV4, FIZZX or MAMMX protein to bind to an intracellular target protein or biologically active portion thereof.

In yet another embodiment, a mutant NOVX protein can be assayed for the ability to regulate a specific biological function (e.g., regulation of exocytosis in exocrine tissues).

Antisense Polynucleotides

Another aspect of the invention pertains to isolated antisense nucleic acid molecules that are hybridizable to or complementary to the nucleic acid molecule comprising the nucleotide sequence of SEQ ID NO: 1, 3, 5, 7, 9, 11, 13, 14 or 16, or 18, 20, 22, 24 or 26 or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire NOV1, NOV2, NOV3, NOV4, FIZZX or MAMMX coding strand, or to only a portion thereof. Nucleic acid molecules encoding fragments, homologs, derivatives and analogs of a NOV1, NOV2, NOV3, NOV4, FIZZX or MAMMX protein, or antisense nucleic acids complementary to a NOV1, NOV2, NOV3, NOV4, FIZZX or MAMMX nucleic acid sequence of SEQ ID NO: 1, 3, 5, 7, 9, 11, 13, 14 or 16, or 18, 20, 22, 24 or 26 are additionally provided.

In one embodiment, an antisense nucleic acid molecule is antisense to a “coding region” of the coding strand of a nucleotide sequence encoding NOV1, NOV2, NOV3, NOV4, FIZZX or MAMMX. The term “coding region” refers to the region of the nucleotide sequence comprising codons which are translated into amino acid residues (e.g., the coding region of human NOV1, NOV2, NOV3, NOV4, FIZZX or MAMMX corresponds to nucleotides 148 to 402 of SEQ ID NO: 1, corresponds to nucleotides 209 to 610 of SEQ ID NO: 3, corresponds to nucleotides 129 to 534 of SEQ ID NO: 5, corresponds to nucleotides 301 to 1084 of SEQ ID NO: 7, and corresponds to nucleotides 341 to 539 of SEQ ID NO: 9. In another embodiment, the antisense nucleic acid molecule is antisense to a “noncoding region” of the coding strand of a nucleotide sequence encoding NOV1, NOV2, NOV3, NOV4, FIZZX or MAMMX. The term “noncoding region” refers to 5′ and 3′ sequences which flank the coding region that are not translated into amino acids (i.e., also referred to as 5′ and 3′ untranslated regions).

Given the coding strand sequences encoding NOV1, NOV2, NOV3, NOV4, FIZZX or MAMMX disclosed herein (e.g., SEQ ID NO: 1, 3, 5, 7, 9, 11, 13, 14 or 16, or 18, 20, 22, 24 or 26), antisense nucleic acids of the invention can be designed according to the rules of Watson and Crick or Hoogsteen base pairing. The antisense nucleic acid molecule can be complementary to the entire coding region of NOV1, NOV2, NOV3, NOV4, FIZZX or MAMMX mRNA, but more preferably is an oligonucleotide that is antisense to only a portion of the coding or noncoding region of NOV1, NOV2, NOV3, NOV4, FIZZX or MAMMX mRNA. For example, the antisense oligonucleotide can be complementary to the region surrounding the translation start site of NOV1, NOV2, NOV3, NOV4, FIZZX or MAMMX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOV1, NOV2, NOV3, NOV4, FIZZX or MAMMX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an a-anomeric nucleic acid molecule. An a-anomeric nucleic acid molecule forms specific double-stranded hybrids with complementary RNA in which, contrary to the usual b-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA -DNA analogue (Inoue et al. (1987) FEBS Lett 215: 327-330).

Ribozymes and PNA moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave NOV1, NOV2, NOV3, NOV4, FIZZX or MAMMX mRNA transcripts to thereby inhibit translation of NOV1, NOV2, NOV3, NOV4, FIZZX or MAMMX mRNA. A ribozyme having specificity for a NOV1, NOV2, NOV3, NOV4, FIZZX or MAMMX -encoding nucleic acid can be designed based upon the nucleotide sequence of a NOV1, NOV2, NOV3, NOV4, FIZZX or MAMMX cDNA disclosed herein (i.e., SEQ ID NO: 1, 3, 5, 7, 9, 11, 13, 14 or 16, or 18, 20, 22, 24 or 26). For example, a derivative of a Tetrahymena L-19 IVS RNA can be constructed in which the nucleotide sequence of the active site is complementary to the nucleotide sequence to be cleaved in a NOV1, NOV2, NOV3, NOV4, FIZZX or MAMMX -encoding mRNA. See, e.g., Cech et al. U.S. Pat. No. 4,987,071; and Cech et al. U.S. Pat. No. 5,116,742. Alternatively, NOV1, NOV2, NOV3, NOV4, FIZZX or MAMMX mRNA can be used to select a catalytic RNA having a specific ribonuclease activity from a pool of RNA molecules. See, e.g., Bartel et al., (1993) Science 261:1411-1418.

Alternatively, NOV1, NOV2, NOV3, NOV4, FIZZX or MAMMX gene expression can be inhibited by targeting nucleotide sequences complementary to the regulatory region of the NOV1, NOV2, NOV3, NOV4, FIZZX or MAMMX (e.g., the NOV1, NOV2, NOV3, NOV4, FIZZX or MAMMX promoter and/or enhancers) to form triple helical structures that prevent transcription of the NOV1, NOV2, NOV3, NOV4, FIZZX or MAMMX gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of NOV1, NOV2, NOV3, NOV4, FIZZX or MAMMX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of NOV1, NOV2, NOV3, NOV4, FIZZX or MAMMX can be used in therapeutic and diagnostic applications. For example, PNAs can be used as antisense or antigene agents for sequence-specific modulation of gene expression by, e.g., inducing transcription or translation arrest or inhibiting replication. PNAs of NOV1, NOV2, NOV3, NOV4, FIZZX or MAMMX can also be used, e.g., in the analysis of single base pair mutations in a gene by, e.g., PNA directed PCR clamping; as artificial restriction enzymes when used in combination with other enzymes, e.g., SI nucleases (Hyrup B. (1996) above); or as probes or primers for DNA sequence and hybridization (Hyrup et al. (1996), above; Perry-O'Keefe (1996), above).

In another embodiment, PNAs of NOV1, NOV2, NOV3, NOV4, FIZZX or MAMMX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOV1, NOV2, NOV3, NOV4, FIZZX or MAMMX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810) or the blood-brain barrier (see, e.g., PCT Publication No. W089/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOV1, NOV2, NOV3, NOV4, FIZZX or MAMMX Proteins

A polypeptide according to the invention includes a polypeptide including the amino acid sequence of NOVX polypeptides whose sequences are provided in SEQ ID NOS: 2n, wherein n is an integer between 1 and 45. The invention also includes a mutant or variant protein any of whose residues may be changed from the corresponding residues shown in SEQ ID NOS: 2n, wherein n is an integer between 1 and 45, while still encoding a protein that maintains its NOVX activities and physiological functions, or a functional fragment thereof.

In general, A NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOV1, NOV2, NOV3, NOV4, FIZZX or MAMMX proteins, and biologically active portions thereof, or derivatives, fragments, analogs or homologs thereof. Also provided are polypeptide fragments suitable for use as immunogens to raise anti-NOV1, NOV2, NOV3, NOV4, FIZZX or MAMMX antibodies. In one embodiment, native NOV1, NOV2, NOV3, NOV4, FIZZX or MAMMX proteins can be isolated from cells or tissue sources by an appropriate purification scheme using standard protein purification techniques. In another embodiment, NOV1, NOV2, NOV3, NOV4, FIZZX or MAMMX proteins are produced by recombinant DNA techniques. Alternative to recombinant expression, a NOV1, NOV2, NOV3, NOV4, FIZZX or MAMMX protein or polypeptide can be synthesized chemically using standard peptide synthesis techniques.

An “isolated” or “purified” protein or biologically active portion thereof is substantially free of cellular material or other contaminating proteins from the cell or tissue source from which the NOV1, NOV2, NOV3, NOV4, FIZZX or MAMMX protein is derived, or substantially free from chemical precursors or other chemicals when chemically synthesized. The language “substantially free of cellular material” includes preparations of NOV1, NOV2, NOV3, NOV4, FIZZX or MAMMX protein in which the protein is separated from cellular components of the cells from which it is isolated or recombinantly produced. In one embodiment, the language “substantially free of cellular material” includes preparations of NOV1, NOV2, NOV3, NOV4, FIZZX or MAMMX protein having less than about 30% (by dry weight) of non-NOV1, NOV2, NOV3, NOV4, FIZZX or MAMMX protein (also referred to herein as a “contaminating protein”), more preferably less than about 20% of non-NOV1, NOV2, NOV3, NOV4, FIZZX or MAMMX protein, still more preferably less than about 10% of non-NOV1, NOV2, NOV3, NOV4, FIZZX or MAMMX protein, and most preferably less than about 5% non-NOV1, NOV2, NOV3, NOV4, FIZZX or MAMMX protein. When the NOV1, NOV2, NOV3, NOV4, FIZZX or MAMMX protein or biologically active portion thereof is recombinantly produced, it is also preferably substantially free of culture medium, i e., culture medium represents less than about 20%, more preferably less than about 10%, and most preferably less than about 5% of the volume of the protein preparation.

The language “substantially free of chemical precursors or other chemicals” includes preparations of NOV1, NOV2, NOV3, NOV4, FIZZX or MAMMX protein in which the protein is separated from chemical precursors or other chemicals that are involved in the synthesis of the protein. In one embodiment, the language “substantially free of chemical precursors or other chemicals” includes preparations of NOV1, NOV2, NOV3, NOV4, FIZZX or MAMMX protein having less than about 30% (by dry weight) of chemical precursors or non-NOV1, NOV2, NOV3, NOV4, FIZZX or MAMMX chemicals, more preferably less than about 20% chemical precursors or non-NOV1, NOV2, NOV3, NOV4, FIZZX or MAMMX chemicals, still more preferably less than about 10% chemical precursors or non-NOV1, NOV2, NOV3, NOV4, FIZZX or MAMMX chemicals, and most preferably less than about 5% chemical precursors or non-NOV1, NOV2, NOV3, NOV4, FIZZX or MAMMX chemicals.

Biologically active portions of a NOV1, NOV2, NOV3, NOV4, FIZZX or MAMMX protein include peptides comprising amino acid sequences sufficiently homologous to or derived from the amino acid sequence of the NOV1, NOV2, NOV3, NOV4, FIZZX or MAMMX protein, e.g., the amino acid sequence shown in SEQ ID NO: 2, 4, 6, 8, 10, that include fewer amino acids than the full length NOV1, NOV2, NOV3, NOV4, FIZZX or MAMMX proteins, and exhibit at least one activity of a NOV1, NOV2, NOV3, NOV4, FIZZX or MAMMX protein. Typically, biologically active portions comprise a domain or motif with at least one activity of the NOV1, NOV2, NOV3, NOV4, FIZZX or MAMMX protein. A biologically active portion of a NOV1, NOV2, NOV3, NOV4, FIZZX or MAMMX protein can be a polypeptide which is, for example, 10, 25, 50, 100 or more amino acids in length.

Moreover, other biologically active portions, in which other regions of the protein are deleted, can be prepared by recombinant techniques and evaluated for one or more of the functional activities of a native NOV1, NOV2, NOV3, NOV4, FIZZX or MAMMX protein.

In an embodiment, the NOV1, NOV2, NOV3, NOV4, FIZZX or MAMMX protein has an amino acid sequence shown in SEQ ID NO: 2, 4, 6, 8, 10. In other embodiments, the NOV1, NOV2, NOV3, NOV4, FIZZX or MAMMX protein is substantially homologous to SEQ ID NO: 2, 4, 6, 8, 10 and retains the functional activity of the protein of SEQ ID NO: 2, 4, 6, 8, 10 yet differs in amino acid sequence due to natural allelic variation or mutagenesis, as described in detail below. Accordingly, in another embodiment, the NOV1, NOV2, NOV3, NOV4, FIZZX or MAMMX protein is a protein that comprises an amino acid sequence at least about 45% homologous to the amino acid sequence of SEQ ID NO: 2, 4, 6, 8, 10 and retains the functional activity of the NOV1, NOV2, NOV3, NOV4, FIZZX or MAMMX proteins of SEQ ID NO: 2, 4, 6, 8, 10

Determining similarity between two or more sequences

To determine the percent similarity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “similarity” is equivalent to amino acid or nucleic acid “identity”).

The nucleic acid sequence similarity may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO: 1, 3, 5, 7, 9.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and fusion proteins

The invention also provides NOV1, NOV2, NOV3, NOV4, FIZZX or MAMMX chimeric or fusion proteins. As used herein, a NOV1, NOV2, NOV3, NOV4, FIZZX or MAMMX “chimeric protein” or “fusion protein” comprises a NOV1, NOV2, NOV3, NOV4, FIZZX or MAMMX polypeptide operatively linked to a non-NOV1, NOV2, NOV3, NOV4, FIZZX or MAMMX polypeptide. A “NOV1, NOV2, NOV3, NOV4, FIZZX or MAMMX polypeptide” refers to a polypeptide having an amino acid sequence corresponding to NOV1, NOV2, NOV3, NOV4, FIZZX or MAMMX, whereas a “non-NOV1, NOV2, NOV3, NOV4, FIZZX or MAMMX polypeptide” refers to a polypeptide having an amino acid sequence corresponding to a protein that is not substantially homologous to the NOV1, NOV2, NOV3, NOV4, FIZZX or MAMMX protein, e.g., a protein that is different from the NOV1, NOV2, NOV3, NOV4, FIZZX or MAMMX protein and that is derived from the same or a different organism. Within a NOV1, NOV2, NOV3, NOV4, FIZZX or MAMMX fusion protein the NOV1, NOV2, NOV3, NOV4, FIZZX or MAMMX polypeptide can correspond to all or a portion of a NOV1, NOV2, NOV3, NOV4, FIZZX or MAMMX protein. In one embodiment, a NOV1, NOV2, NOV3, NOV4, FIZZX or MAMMX fusion protein comprises at least one biologically active portion of a NOV1, NOV2, NOV3, NOV4, FIZZX or MAMMX protein. In another embodiment, a NOV1, NOV2, NOV3, NOV4, FIZZX or MAMMX fusion protein comprises at least two biologically active portions of a NOV1, NOV2, NOV3, NOV4, FIZZX or MAMMX protein. In yet another embodiment, a NOV1, NOV2, NOV3, NOV4, FIZZX or MAMMX fusion protein comprises at least three biologically active portions of a NOV1, NOV2, NOV3, NOV4, FIZZX or MAMMX protein. Within the fusion protein, the term “operatively linked” is intended to indicate that the NOV1, NOV2, NOV3, NOV4, FIZZX or MAMMX polypeptide and the non-NOV1, NOV2, NOV3, NOV4, FIZZX or MAMMX polypeptide are fused in-frame to each other. The non-NOV1, NOV2, NOV3, NOV4, FIZZX or MAMMX polypeptide can be fused to the N-terminus or C-terminus of the NOV1, NOV2, NOV3, NOV4, FIZZX or MAMMX polypeptide.

In certain embodiments a NOV1, NOV2, NOV3, NOV4, FIZZX or MAMMX fusion protein comprises a NOV1, NOV2, NOV3, NOV4, FIZZX or MAMMX domain linked to the extracellular domain of a second protein. Such fusion proteins can be further utilized in screening assays for compounds which modulate NOV1, NOV2, NOV3, NOV4, FIZZX or MAMMX activity.

In yet another embodiment, the fusion protein is a GST-NOV1, NOV2, NOV3, NOV4, FIZZX or MAMMX fusion protein in which the NOV1, NOV2, NOV3, NOV4, FIZZX or MAMMX sequences are fused to the C-terminus of the GST (i.e., glutathione S-transferase) sequences. Such fusion proteins can facilitate the purification of recombinant NOV1, NOV2, NOV3, NOV4, FIZZX or MAMMX.

In another embodiment, the fusion protein is a NOV1, NOV2, NOV3, NOV4, FIZZX or MAMMX protein containing a heterologous signal sequence at its N-terminus. For example, the native NOV1, NOV2, NOV3, NOV4, FIZZX or MAMMX signal sequence can be removed and replaced with a signal sequence from another protein. In certain host cells (e.g., mammalian host cells), expression and/or secretion of NOV1, NOV2, NOV3, NOV4, FIZZX or MAMMX can be increased through use of a heterologous signal sequence.

In yet another embodiment, the fusion protein is a NOV1, NOV2, NOV3, NOV4, FIZZX or MAMMX -immunoglobulin fusion protein in which the NOV1, NOV2, NOV3, NOV4, FIZZX or MAMMX sequences are fused to sequences derived from a member of the immunoglobulin protein family. The NOV1, NOV2, NOV3, NOV4, FIZZX or MAMMX -immunoglobulin fusion proteins of the invention can be incorporated into pharmaceutical compositions and administered to a subject to inhibit an interaction between a NOV1, NOV2, NOV3, NOV4, FIZZX or MAMMX ligand and a NOV1, NOV2, NOV3, NOV4, FIZZX or MAMMX protein on the surface of a cell, to thereby suppress NOV1, NOV2, NOV3, NOV4, FIZZX or MAMMX -mediated signal transduction in vivo. The NOV1, NOV2, NOV3, NOV4, FIZZX or MAMMX -immunoglobulin fusion proteins can be used to affect the bioavailability of a NOV1, NOV2, NOV3, NOV4, FIZZX or MAMMX cognate ligand. Inhibition of the NOV1, NOV2, NOV3, NOV4, FIZZX or MAMMX ligand/NOV1, NOV2, NOV3, NOV4, FIZZX or MAMMX interaction may be useful therapeutically for both the treatment of proliferative and differentiative disorders, as well as modulating (e.g. promoting or inhibiting) cell survival. Moreover, the NOV1, NOV2, NOV3, NOV4, FIZZX or MAMMX -immunoglobulin fusion proteins of the invention can be used as immunogens to produce anti-NOV1, NOV2, NOV3, NOV4, FIZZX or MAMMX antibodies in a subject, to purify NOV1, NOV2, NOV3, NOV4, FIZZX or MAMMX ligands, and in screening assays to identify molecules that inhibit the interaction of NOV1, NOV2, NOV3, NOV4, FIZZX or MAMMX with a NOV1, NOV2, NOV3, NOV4, FIZZX or MAMMX ligand.

A NOV1, NOV2, NOV3, NOV4, FIZZX or MAMMX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOV1, NOV2, NOV3, NOV4, FIZZX or MAMMX -encoding nucleic acid can be cloned into such an expression vector such that the fusion moiety is linked in-frame to the NOV1, NOV2, NOV3, NOV4, FIZZX or MAMMX protein.

Agonists and Antagonists

The invention also pertains to variants of the NOV1, NOV2, NOV3, NOV4, FIZZX or MAMMX proteins that function as either NOV1, NOV2, NOV3, NOV4, FIZZX or MAMMX agonists (mimetics) or as NOV1, NOV2, NOV3, NOV4, FIZZX or MAMMX antagonists.

Variants of the NOV1, NOV2, NOV3, NOV4, FIZZX or MAMMX protein can be generated by mutagenesis, e.g., discrete point mutation or truncation of the NOV1, NOV2, NOV3, NOV4, FIZZX or MAMMX protein. An agonist of the NOV1, NOV2, NOV3, NOV4, FIZZX or MAMMX protein can retain substantially the same, or a subset of, the biological activities of the naturally occurring form of the NOV1, NOV2, NOV3, NOV4, FIZZX or MAMMX protein. An antagonist of the NOV1, NOV2, NOV3, NOV4, FIZZX or MAMMX protein can inhibit one or more of the activities of the naturally occurring form of the NOV1, NOV2, NOV3, NOV4, FIZZX or MAMMX protein by, for example, competitively binding to a downstream or upstream member of a cellular signaling cascade which includes the NOV1, NOV2, NOV3, NOV4, FIZZX or MAMMX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOV1, NOV2, NOV3, NOV4, FIZZX or MAMMX proteins.

Variants of the NOV1, NOV2, NOV3, NOV4, FIZZX or MAMMX protein that function as either NOV1, NOV2, NOV3, NOV4, FIZZX or MAMMX agonists (mimetics) or as NOV1, NOV2, NOV3, NOV4, FIZZX or MAMMX antagonists can be identified by screening combinatorial libraries of mutants, e.g., truncation mutants, of the NOV1, NOV2, NOV3, NOV4, FIZZX or MAMMX protein for NOV1, NOV2, NOV3, NOV4, FIZZX or MAMMX protein agonist or antagonist activity. In one embodiment, a variegated library of NOV1, NOV2, NOV3, NOV4, FIZZX or MAMMX variants is generated by combinatorial mutagenesis at the nucleic acid level and is encoded by a variegated gene library. A variegated library of NOV1, NOV2, NOV3, NOV4, FIZZX or MAMMX variants can be produced by, for example, enzymatically ligating a mixture of synthetic oligonucleotides into gene sequences such that a degenerate set of potential NOV1, NOV2, NOV3, NOV4, FIZZX or MAMMX sequences is expressible as individual polypeptides, or alternatively, as a set of larger fusion proteins (e.g., for phage display) containing the set of NOV1, NOV2, NOV3, NOV4, FIZZX or MAMMX sequences therein. There are a variety of methods which can be used to produce libraries of potential NOV1, NOV2, NOV3, NOV4, FIZZX or MAMMX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOV1, NOV2, NOV3, NOV4, FIZZX or MAMMX sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide libraries

In addition, libraries of fragments of the NOV1, NOV2, NOV3, NOV4, FIZZX or MAMMX protein coding sequence can be used to generate a variegated population of NOV1, NOV2, NOV3, NOV4, FIZZX or MAMMX fragments for screening and subsequent selection of variants of a NOV1, NOV2, NOV3, NOV4, FIZZX or MAMMX protein. In one embodiment, a library of coding sequence fragments can be generated by treating a double stranded PCR fragment of a NOV1, NOV2, NOV3, NOV4, FIZZX or MAMMX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S I nuclease, and ligating the resulting fragment library into an expression vector. By this method, an expression library can be derived which encodes N-terminal and internal fragments of various sizes of the NOV1, NOV2, NOV3, NOV4, FIZZX or MAMMX protein.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOV1, NOV2, NOV3, NOV4, FIZZX or MAMMX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique that enhances the frequency of functional mutants in the libraries, can be used in combination with the screening assays to identify NOV1, NOV2, NOV3, NOV4, FIZZX or MAMMX variants (Arkin and Yourvan (1992) PNAS 89:7811-7815; Delgrave et al. (1993) Protein Engineering 6:327-331).

Anti-NOV1, NOV2, NOV3, NOV4, FIZZX or MAMMX antibodies The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab′}and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOV1, NOV2, NOV3, NOV4, FIZZX or MAMMX protein, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NOs: 2n, wherein n is an integer between 1 and 45, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (BLISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOV1, NOV2, NOV3, NOV4, FIZZX or MAMMX Recombinant Expression Vectors and Host Cells

Another aspect of the invention pertains to vectors, preferably expression vectors, containing a nucleic acid encoding NOV1, NOV2, NOV3, NOV4, FIZZX or MAMMX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOV1, NOV2, NOV3, NOV4, FIZZX or MAMMX proteins, mutant forms of NOV1, NOV2, NOV3, NOV4, FIZZX or MAMMX, fusion proteins, etc.).

The recombinant expression vectors of the invention can be designed for expression of NOV1, NOV2, NOV3, NOV4, FIZZX or MAMMX in prokaryotic or eukaryotic cells. For example, NOV1, NOV2, NOV3, NOV4, FIZZX or MAMMX can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the NOV1, NOV2, NOV3, NOV4, FIZZX or MAMMX expression vector is a yeast expression vector. Examples of vectors for expression in yeast S. cerivisae include pYepSec1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, NOV1, NOV2, NOV3, NOV4, FIZZX or MAMMX can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the a-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to NOV1, NOV2, NOV3, NOV4, FIZZX or MAMMX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOV1, NOV2, NOV3, NOV4, FIZZX or MAMMX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOV1, NOV2, NOV3, NOV4, FIZZX or MAMMX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOV1, NOV2, NOV3, NOV4, FIZZX or MAMMX protein. Accordingly, the invention further provides methods for producing NOV1, NOV2, NOV3, NOV4, FIZZX or MAMMX protein using the host cells of the invention. In one embodiment, the method comprises culturing the host cell of invention (into which a recombinant expression vector encoding NOV1, NOV2, NOV3, NOV4, FIZZX or MAMMX has been introduced) in a suitable medium such that NOV1, NOV2, NOV3, NOV4, FIZZX or MAMMX protein is produced. In another embodiment, the method further comprises isolating NOV1, NOV2, NOV3, NOV4, FIZZX or MAMMX from the medium or the host cell.

Transgenic animals

The host cells of the invention can also be used to produce nonhuman transgenic animals. For example, in one embodiment, a host cell of the invention is a fertilized oocyte or an embryonic stem cell into which NOV1, NOV2, NOV3, NOV4, FIZZX or MAMMX-coding sequences have been introduced. Such host cells can then be used to create non-human transgenic animals in which exogenous NOV1, NOV2, NOV3, NOV4, FIZZX or MAMMX sequences have been introduced into their genome or homologous recombinant animals in which endogenous NOV1, NOV2, NOV3, NOV4, FIZZX or MAMMX sequences have been altered. Such animals are useful for studying the function and/or activity of NOV1, NOV2, NOV3, NOV4, FIZZX or MAMMX and for identifying and/or evaluating modulators of NOV1, NOV2, NOV3, NOV4, FIZZX or MAMMX activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOV1, NOV2, NOV3, NOV4, FIZZX or MAMMX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOV1, NOV2, NOV3, NOV4, FIZZX or MAMMX -encoding nucleic acid into the male pronuclei of a fertilized oocyte, e.g., by microinjection, retroviral infection, and allowing the oocyte to develop in a pseudopregnant female foster animal. The human NOV1, NOV2, NOV3, NOV4, FIZZX or MAMMX cDNA sequence of SEQ ID NO: 1, 3, 5, 7, 9 can be introduced as a transgene into the genome of a non-human animal. Alternatively, a nonhuman homologue of the human NOV1, NOV2, NOV3, NOV4, FIZZX or MAMMX gene, such as a mouse NOV1, NOV2, NOV3, NOV4, FIZZX or MAMMX gene, can be isolated based on hybridization to the human NOV1, NOV2, NOV3, NOV4, FIZZX or MAMMX cDNA (described further above) and used as a transgene. Intronic sequences and polyadenylation signals can also be included in the transgene to increase the efficiency of expression of the transgene. A tissue-specific regulatory sequence(s) can be operably linked to the NOV1, NOV2, NOV3, NOV4, FIZZX or MAMMX transgene to direct expression of NOV1, NOV2, NOV3, NOV4, FIZZX or MAMMX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOV1, NOV2, NOV3, NOV4, FIZZX or MAMMX transgene in its genome and/or expression of NOV1, NOV2, NOV3, NOV4, FIZZX or MAMMX mRNA in tissues or cells of the animals. A transgenic founder animal can then be used to breed additional animals carrying the transgene. Moreover, transgenic animals carrying a transgene encoding NOV1, NOV2, NOV3, NOV4, FIZZX or MAMMX can further be bred to other transgenic animals carrying other transgenes.

To create a homologous recombinant animal, a vector is prepared which contains at least a portion of a NOV1, NOV2, NOV3, NOV4, FIZZX or MAMMX gene into which a deletion, addition or substitution has been introduced to thereby alter, e.g., functionally disrupt, the NOV1, NOV2, NOV3, NOV4, FIZZX or MAMMX gene. The NOV1, NOV2, NOV3, NOV4, FIZZX or MAMMX gene can be a human gene (e.g., the cDNA of SEQ ID NO: 1, 3, 5, 7, 9) but more preferably, is a non-human homologue of a human NOV1, NOV2, NOV3, NOV4, FIZZX or MAMMX gene. For example, a mouse homologue of human NOV1, NOV2, NOV3, NOV4, FIZZX or MAMMX gene of SEQ ID NO: 1, 3, 5, 7, 9 can be used to construct a homologous recombination vector suitable for altering an endogenous NOV1, NOV2, NOV3, NOV4, FIZZX or MAMMX gene in the mouse genome. In one embodiment, the vector is designed such that, upon homologous recombination, the endogenous NOV1, NOV2, NOV3, NOV4, FIZZX or MAMMX gene is functionally disrupted (i.e., no longer encodes a functional protein; also referred to as a “knock out” vector).

Alternatively, the vector can be designed such that, upon homologous recombination, the endogenous NOV1, NOV2, NOV3, NOV4, FIZZX or MAMMX gene is mutated or otherwise altered but still encodes functional protein (e.g., the upstream regulatory region can be altered to thereby alter the expression of the endogenous NOV1, NOV2, NOV3, NOV4, FIZZX or MAMMX protein). In the homologous recombination vector, the altered portion of the NOV1, NOV2, NOV3, NOV4, FIZZX or MAMMX gene is flanked at its 5′ and 3′ ends by additional nucleic acid of the NOV1, NOV2, NOV3, NOV4, FIZZX or MAMMX gene to allow for homologous recombination to occur between the exogenous NOV1, NOV2, NOV3, NOV4, FIZZX or MAMMX gene carried by the vector and an endogenous NOV1, NOV2, NOV3, NOV4, FIZZX or MAMMX gene in an embryonic stem cell. The additional flanking NOV1, NOV2, NOV3, NOV4, FIZZX or MAMMX nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51:503 for a description of homologous recombination vectors. The vector is introduced into an embryonic stem cell line (e.g., by electroporation) and cells in which the introduced NOV1, NOV2, NOV3, NOV4, FIZZX or MAMMX gene has homologously recombined with the endogenous NOV1, NOV2, NOV3, NOV4, FIZZX or MAMMX gene are selected (see e.g., Li et al. (1992) Cell 69:915).

The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2:823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Pharmaceutical Compositions

The NOV1, NOV2, NOV3, NOV4, FIZZX or MAMMX nucleic acid molecules, NOV1, NOV2, NOV3, NOV4, FIZZX or MAMMX proteins, and anti-FIZZX, anti-NOV1, anti-NOV2, anti-NOV3, anti-NOV4, or anti-Mamm-X antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., aNOV1, NOV2, NOV3, NOV4, FIZZX or MAMMX protein or anti-NOV1, NOV2, NOV3, NOV4, FIZZX or MAMMX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS.91 :3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Uses and Methods of the Invention

The nucleic acid molecules, proteins, protein homologues, and antibodies described herein can be used in one or more of the following methods: (a) screening assays; (b) detection assays (e.g., chromosomal mapping, tissue typing, forensic biology), (c) predictive medicine (e.g., diagnostic assays, prognostic assays, monitoring clinical trials, and pharmacogenomics); and (d) methods of treatment (e.g., therapeutic and prophylactic). A NOV1, NOV2, NOV3, NOV4, FIZZX or MAMMX protein interacts with other cellular proteins and can thus be used to (i) modulation of protein activity; (ii) regulation of cellular proliferation; (iii) regulation of cellular differentiation; and (iv) regulation of cell survival.

The isolated nucleic acid molecules of the invention can be used to express NOV1, NOV2, NOV3, NOV4, FIZZX or MAMMX protein (e.g., via a recombinant expression vector in a host cell in gene therapy applications), to detect NOV1, NOV2, NOV3, NOV4, FIZZX or MAMMX mRNA (e.g., in a biological sample) or a genetic lesion in a NOV1, NOV2, NOV3, NOV4, FIZZX or MAMMX gene, and to modulate NOV1, NOV2, NOV3, NOV4, FIZZX or MAMMX activity, as described further below. In addition, the NOV1, NOV2, NOV3, NOV4, FIZZX or MAMMX proteins can be used to screen drugs or compounds that modulate the NOV1, NOV2, NOV3, NOV4, FIZZX or MAMMX activity or expression as well as to treat disorders characterized by insufficient or excessive production of NOV1, NOV2, NOV3, NOV4, FIZZX or MAMMX protein or production of NOV1, NOV2, NOV3, NOV4, FIZZX or MAMMX protein forms that have decreased or aberrant activity compared to NOV1, NOV2, NOV3, NOV4, FIZZX or MAMMX wild type protein (e.g. proliferative disorders such as cancer or preclampsia). In addition, the anti-NOV1, NOV2, NOV3, NOV4, FIZZX or MAMMX antibodies of the invention can be used to detect and isolate NOV1, NOV2, NOV3, NOV4, FIZZX or MAMMX proteins and modulate NOV1, NOV2, NOV3, NOV4, FIZZX or MAMMX activity.

This invention further pertains to novel agents identified by the above described screening assays and uses thereof for treatments as described herein.

Screening Assays

The invention provides a method (also referred to herein as a “screening assay”) for identifying modulators, i.e., candidate or test compounds or agents (e.g., peptides, peptidomimetics, small molecules or other drugs) that bind to NOV1, NOV2, NOV3, NOV4, FIZZX or MAMMX proteins or have a stimulatory or inhibitory effect on, for example, NOV1, NOV2, NOV3, NOV4, FIZZX or MAMMX expression or NOV1, NOV2, NOV3, NOV4, FIZZX or MAMMX activity.

In one embodiment, the invention provides assays for screening candidate or test compounds which bind to or modulate the activity of the membrane-bound form of a NOV1, NOV2, NOV3, NOV4, FIZZX or MAMMX protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994)J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Biol 222:301-310; Ladner above.).

In one embodiment, an assay is a cell-based assay in which a cell which expresses a membrane-bound form of NOV1, NOV2, NOV3, NOV4, FIZZX or MAMMX protein, or a biologically active portion thereof, on the cell surface is contacted with a test compound and the ability of the test compound to bind to a NOV1, NOV2, NOV3, NOV4, FIZZX or MAMMX protein determined. The cell, for example, can of mammalian origin or a yeast cell. Determining the ability of the test compound to bind to the NOV1, NOV2, NOV3, NOV4, FIZZX or MAMMX protein can be accomplished, for example, by coupling the test compound with a radioisotope or enzymatic label such that binding of the test compound to the NOV1, NOV2, NOV3, NOV4, FIZZX or MAMMX protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOV1, NOV2, NOV3, NOV4, FIZZX or MAMMX protein, or a biologically active portion thereof, on the cell surface with a known compound which binds NOV1, NOV2, NOV3, NOV4, FIZZX or MAMMX to form an assay mixture, contacting the assay mixture with a test compound, and determining the ability of the test compound to interact with a NOV1, NOV2, NOV3, NOV4, FIZZX or MAMMX protein, wherein determining the ability of the test compound to interact with a NOV1, NOV2, NOV3, NOV4, FIZZX or MAMMX protein comprises determining the ability of the test compound to preferentially bind to NOV1, NOV2, NOV3, NOV4, FIZZX or MAMMX or a biologically active portion thereof as compared to the known compound.

In another embodiment, an assay is a cell-based assay comprising contacting a cell expressing a membrane-bound form of NOV1, NOV2, NOV3, NOV4, FIZZX or MAMMX protein, or a biologically active portion thereof, on the cell surface with a test compound and determining the ability of the test compound to modulate (e.g., stimulate or inhibit) the activity of the NOV1, NOV2, NOV3, NOV4, FIZZX or MAMMX protein or biologically active portion thereof. Determining the ability of the test compound to modulate the activity of NOV1, NOV2, NOV3, NOV4, FIZZX or MAMMX or a biologically active portion thereof can be accomplished, for example, by determining the ability of the NOV1, NOV2, NOV3, NOV4, FIZZX or MAMMX protein to bind to or interact with a NOV1, NOV2, NOV3, NOV4, FIZZX or MAMMX target molecule. As used herein, a “target molecule” is a molecule with which a NOV1, NOV2, NOV3, NOV4, FIZZX or MAMMX protein binds or interacts in nature, for example, a molecule on the surface of a cell which expresses a NOV1, NOV2, NOV3, NOV4, FIZZX or MAMMX protein, a molecule on the surface of a second cell, a molecule in the extracellular milieu, a molecule associated with the internal surface of a cell membrane or a cytoplasmic molecule. A NOV1, NOV2, NOV3, NOV4, FIZZX or MAMMX target molecule can be a non-NOV1, NOV2, NOV3, NOV4, FIZZX or MAMMX molecule or a NOV1, NOV2, NOV-3, NOV4, FIZZX or MAMMX protein or polypeptide of the present invention. In one embodiment, a NOV1, NOV2, NOV3, NOV4, FIZZX or MAMMX target molecule is a component of a signal transduction pathway that facilitates transduction of an extracellular signal (e.g. a signal generated by binding of a compound to a membrane-bound NOV1, NOV2, NOV3, NOV4, FIZZX or MAMMX molecule) through the cell membrane and into the cell. The target, for example, can be a second intercellular protein that has catalytic activity or a protein that facilitates the association of downstream signaling molecules with NOV1, NOV2, NOV3, NOV4, FIZZX or MAMMX.

Determining the ability of the NOV1, NOV2, NOV3, NOV4, FIZZX or MAMMX protein to bind to or interact with a NOV1, NOV2, NOV3, NOV4, FIZZX or MAMMX target molecule can be accomplished by one of the methods described above for determining direct binding. In one embodiment, determining the ability of the NOV1, NOV2, NOV3, NOV4, FIZZX or MAMMX protein to bind to or interact with a NOV1, NOV2, NOV3, NOV4, FIZZX or MAMMX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOV1, NOV2, NOV3, NOV4, FIZZX or MAMMX -responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a NOV1, NOV2, NOV3, NOV4, FIZZX or MAMMX protein or biologically active portion thereof with a test compound and determining the ability of the test compound to bind to the NOV1, NOV2, NOV3, NOV4, FIZZX or MAMMX protein or biologically active portion thereof. Binding of the test compound to the NOV1, NOV2, NOV3, NOV4, FIZZX or MAMMX protein can be determined either directly or indirectly as described above. In one embodiment, the assay comprises contacting the NOV1, NOV2, NOV3, NOV4, FIZZX or MAMMX protein or biologically active portion thereof with a known compound which binds NOV1, NOV2, NOV3, NOV4, FIZZX or MAMMX to form an assay mixture, contacting the assay mixture with a test compound, and determining the ability of the test compound to interact with a NOV1, NOV2, NOV3, NOV4, FIZZX or MAMMX protein, wherein determining the ability of the test compound to interact with a NOV1, NOV2, NOV3, NOV4, FIZZX or MAMMX protein comprises determining the ability of the test compound to preferentially bind to NOV1, NOV2, NOV3, NOV4, FIZZX or MAMMX or biologically active portion thereof as compared to the known compound.

In another embodiment, an assay is a cell-free assay comprising contacting NOV1, NOV2, NOV3, NOV4, FIZZX or MAMMX protein or biologically active portion thereof with a test compound and determining the ability of the test compound to modulate (e.g. stimulate or inhibit) the activity of the NOV1, NOV2, NOV3, NOV4, FIZZX or MAMMX protein or biologically active portion thereof. Determining the ability of the test compound to modulate the activity of NOV1, NOV2, NOV3, NOV4, FIZZX or MAMMX can be accomplished, for example, by determining the ability of the NOV1, NOV2, NOV3, NOV4, FIZZX or MAMMX protein to bind to a NOV1, NOV2, NOV3, NOV4, FIZZX or MAMMX target molecule by one of the methods described above for determining direct binding. In an alternative embodiment, determining the ability of the test compound to modulate the activity of NOV1, NOV2, NOV3, NOV4, FIZZX or MAMMX can be accomplished by determining the ability of the NOV1, NOV2, NOV3, NOV4, FIZZX or MAMMX protein further modulate a NOV1, NOV2, NOV3, NOV4, FIZZX or MAMMX target molecule. For example, the catalytic/enzymatic activity of the target molecule on an appropriate substrate can be determined as previously described, supra.

In yet another embodiment, the cell-free assay comprises contacting the NOV1, NOV2, NOV3, NOV4, FIZZX or MAMMX protein or biologically active portion thereof with a known compound which binds NOV1, NOV2, NOV3, NOV4, FIZZX or MAMMX to form an assay mixture, contacting the assay mixture with a test compound, and determining the ability of the test compound to interact with a NOV1, NOV2, NOV3, NOV4, FIZZX or MAMMX protein, wherein determining the ability of the test compound to interact with a NOV1, NOV2, NOV3, NOV4, FIZZX or MAMMX protein comprises determining the ability of the NOV1, NOV2, NOV3, NOV4, FIZZX or MAMMX protein to preferentially bind to or modulate the activity of a NOV1, NOV2, NOV3, NOV4, FIZZX or MAMMX target molecule.

The cell-free assays of the present invention are amenable to use of both the soluble form or the membrane-bound form of NOV1, NOV2, NOV3, NOV4, FIZZX or MAMMX. In the case of cell-free assays comprising the membrane-bound form of NOV1, NOV2, NOV3, NOV4, FIZZX or MAMMX , it may be desirable to utilize a solubilizing agent such that the membrane-bound form of NOV1, NOV2, NOV3, NOV4, FIZZX or MAMMX is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Tritono® X-100, Triton® X-114, Thesit®, Isotridecypoly(ethylene glycol ether)ₙ, 3-(3-cholamidopropyl)dimethylamminiol-1-propane sulfonate (CHAPS), 3-(3-cholamidopropyl)dimethylamminiol-2-hydroxy-1-propane sulfonate (CHAPSO), or N-dodecyl—N,N-dimethyl-3-ammonio-1-propane sulfonate.

In more than one embodiment of the above assay methods of the present invention, it may be desirable to immobilize either NOV1, NOV2, NOV3, NOV4, FIZZX or MAMMX or its target molecule to facilitate separation of complexed from uncomplexed forms of one or both of the proteins, as well as to accommodate automation of the assay. Binding of a test compound to NOV1, NOV2, NOV3, NOV4, FIZZX or MAMMX , or interaction of NOV1, NOV2, NOV3, NOV4, FIZZX or MAMMX with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOV1, NOV2, NOV3, NOV4, FIZZX or MAMMX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOV1, NOV2, NOV3, NOV4, FIZZX or MAMMX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of NOV1, NOV2, NOV3, NOV4, FIZZX or MAMMX binding or activity determined using standard techniques.

Other techniques for immobilizing proteins on matrices can also be used in the screening assays of the invention. For example, either NOV1, NOV2, NOV3, NOV4, FIZZX or MAMMX or its target molecule can be immobilized utilizing conjugation of biotin and streptavidin. Biotinylated NOV1, NOV2, NOV3, NOV4, FIZZX or MAMMX or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with NOV1, NOV2, NOV3, NOV4, FIZZX or MAMMX or target molecules, but which do not interfere with binding of the NOV1, NOV2, NOV3, NOV4, FIZZX or MAMMX protein to its target molecule, can be derivatized to the wells of the plate, and unbound target or NOV1, NOV2, NOV3, NOV4, FIZZX or MAMMX trapped in the wells by antibody conjugation. Methods for detecting such complexes, in addition to those described above for the GST-immobilized complexes, include immunodetection of complexes using antibodies reactive with the NOV1, NOV2, NOV3, NOV4, FIZZX or MAMMX or target molecule, as well as enzyme-linked assays that rely on detecting an enzymatic activity associated with the NOV1, NOV2, NOV3, NOV4, FIZZX or MAMMX or target molecule.

In another embodiment, modulators of NOV1, NOV2, NOV3, NOV4, FIZZX or MAMMX expression are identified in a method wherein a cell is contacted with a candidate compound and the expression of NOV1, NOV2, NOV3, NOV4, FIZZX or MAMMX mRNA or protein in the cell is determined. The level of expression of NOV1, NOV2, NOV3, NOV4, FIZZX or MAMMX mRNA or protein in the presence of the candidate compound is compared to the level of expression of NOV1, NOV2, NOV3, NOV4, FIZZX or MAMMX mRNA or protein in the absence of the candidate compound. The candidate compound can then be identified as a modulator of NOV1, NOV2, NOV3, NOV4, FIZZX or MAMMX expression based on this comparison. For example, when expression of NOV1, NOV2, NOV3, NOV4, FIZZX or MAMMX mRNA or protein is greater (statistically significantly greater) in the presence of the candidate compound than in its absence, the candidate compound is identified as a stimulator of NOV1, NOV2, NOV3, NOV4, FIZZX or MAMMX mRNA or protein expression. Alternatively, when expression of NOV1, NOV2, NOV3, NOV4, FIZZX or MAMMX mRNA or protein is less (statistically significantly less) in the presence of the candidate compound than in its absence, the candidate compound is identified as an inhibitor of NOV1, NOV2, NOV3, NOV4, FIZZX or MAMMX mRNA or protein expression. The level of NOV1, NOV2, NOV3, NOV4, FIZZX or MAMMX mRNA or protein expression in the cells can be determined by methods described herein for detecting NOV1, NOV2, NOV3, NOV4, FIZZX or MAMMX mRNA or protein.

In yet another aspect of the invention, the NOV1, NOV2, NOV3, NOV4, FIZZX or MAMMX proteins can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with NOV1, NOV2, NOV3, NOV4, FIZZX or MAMMX (“NOV1, NOV2, NOV3, NOV4, FIZZX or MAMMX -binding proteins” or “NOV1, NOV2, NOV3, NOV4, FIZZX or MAMMX -bp”) and modulate NOV1, NOV2, NOV3, NOV4, FIZZX or MAMMX activity. Such NOV1, NOV2, NOV3, NOV4, FIZZX or MAMMX -binding proteins are also likely to be involved in the propagation of signals by the NOV1, NOV2, NOV3, NOV4, FIZZX or MAMMX proteins as, for example, upstream or downstream elements of the NOV1, NOV2, NOV3, NOV4, FIZZX or MAMMX pathway.

The two-hybrid system is based on the modular nature of most transcription factors, which consist of separable DNA-binding and activation domains. Briefly, the assay utilizes two different DNA constructs. In one construct, the gene that codes for NOV1, NOV2, NOV3, NOV4, FIZZX or MAMMX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOV1, NOV2, NOV3, NOV4, FIZZX or MAMMX -dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOV1, NOV2, NOV3, NOV4, FIZZX or MAMMX.

This invention further pertains to novel agents identified by the above-described screening assays and uses thereof for treatments as described herein.

Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOV1, NOV2, NOV3, NOV4, FIZZX or MAMMX, sequences, described herein, can be used to map the location of the NOV1, NOV2, NOV3, NOV4, FIZZX or MAMMX genes, respectively, on a chromosome. The mapping of the NOV1, NOV2, NOV3, NOV4, FJZZX or MAMMX sequences to chromosomes is an important first step in correlating these sequences with genes associated with disease.

Briefly, NOV1, NOV2, NOV3, NOV4, FIZZX or MAMMX genes can be mapped to chromosomes by preparing PCR primers (preferably 15-25 bp in length) from the NOV1, NOV2, NOV3, NOV4, FIZZX or MAMMX sequences. Computer analysis of the NOV1, NOV2, NOV3, NOV4, FIZZX or MAMMX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOV1, NOV2, NOV3, NOV4, FIZZX or MAMMX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOV1, NOV2, NOV3, NOV4, FIZZX or MAMMX sequences to design oligonucleotide primers, sublocalization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see Verma et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, for example, in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, for example, Egeland et al. (1987) Nature, 325:783-787.

Moreover, differences in the DNA sequences between individuals affected and unaffected with a disease associated with the NOV1, NOV2, NOV3, NOV4, FIZZX or MAMMX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOV1, NOV2, NOV3, NOV4, FIZZX or MAMMX sequences of the present invention can also be used to identify individuals from minute biological samples. In this technique, an individual's genomic DNA is digested with one or more restriction enzymes, and probed on a Southern blot to yield unique bands for identification. The sequences of the present invention are useful as additional DNA markers for RFLP (“restriction fragment length polymorphisms,” described in U.S. Pat. No. 5,272,057).

Furthermore, the sequences of the present invention can be used to provide an alternative technique that determines the actual base-by-base DNA sequence of selected portions of an individual's genome. Thus, the NOV1, NOV2, NOV3, NOV4, FIZZX or MAMMX sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The NOV1, NOV2, NOV3, NOV4, FIZZX or MAMMX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 1, 3, 5, 7, 9 can comfortably provide positive individual identification with a panel of perhaps 10 to 1,000 primers that each yield a noncoding amplified sequence of 100 bases. If predicted coding sequences, such as those in SEQ ID NO: 1, 3, 5, 7, 9 are used, a more appropriate number of primers for positive individual identification would be 500-2,000.

Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining NOV1, NOV2, NOV3, NOV4, FIZZX or MAMMX protein and/or nucleic acid expression as well as NOV1, NOV2, NOV3, NOV4, FIZZX or MAMMX activity, in the context of a biological sample (e.g., blood, serum, cells, tissue) to thereby determine whether an individual is afflicted with a disease or disorder, or is at risk of developing a disorder, associated with aberrant NOV1, NOV2, NOV3, NOV4, FIZZX or MAMMX expression or activity. The invention also provides for prognostic (or predictive) assays for determining whether an individual is at risk of developing a disorder associated with NOV1, NOV2, NOV3, NOV4, FIZZX or MAMMX protein, nucleic acid expression or activity. For example, mutations in a NOV1, NOV2, NOV3, NOV4, FIZZX or MAMMX gene can be assayed in a biological sample. Such assays can be used for prognostic or predictive purpose to thereby prophylactically treat an individual prior to the onset of a disorder characterized by or associated with NOV1, NOV2, NOV3, NOV4, FIZZX or MAMMX protein, nucleic acid expression or activity.

Another aspect of the invention provides methods for determining NOV1, NOV2, NOV3, NOV4, FIZZX or MAMMX protein, nucleic acid expression or NOV1, NOV2, NOV3, NOV4, FIZZX or MAMMX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOV1, NOV2, NOV3, NOV4, FIZZX or MAMMX in clinical trials.

These and other agents are described in further detail in the following sections.

Diagnostic Assays

An exemplary method for detecting the presence or absence of NOV1, NOV2, NOV3, NOV4, FIZZX or MAMMX in a biological sample involves obtaining a biological sample from a test subject and contacting the biological sample with a compound or an agent capable of detecting NOV1, NOV2, NOV3, NOV4, FIZZX or MAMMX protein or nucleic acid (e.g., mRNA, genomic DNA) that encodes NOV1, NOV2, NOV3, NOV4, FIZZX or MAMMX protein such that the presence of NOV1, NOV2, NOV3, NOV4, FIZZX or MAMMX is detected in the biological sample. An agent for detecting NOV1, NOV2, NOV3, NOV4, FIZZX or MAMMX mRNA or genomic DNA is a labeled nucleic acid probe capable of hybridizing to NOV1, NOV2, NOV3, NOV4, FIZZX or MAMMX mRNA or genomic DNA. The nucleic acid probe can be, for example, a full-length NOV1, NOV2, NOV3, NOV4, FIZZX or MAMMX nucleic acid, such as the nucleic acid of SEQ ID NO: 1, 3, 5, 7, 9, or a portion thereof, such as an oligonucleotide of at least 15, 30, 50, 100, 250 or 500 nucleotides in length and sufficient to specifically hybridize under stringent conditions to NOV1, NOV2, NOV3, NOV4, FIZZX or MAMMX mRNA or genomic DNA. Other suitable probes for use in the diagnostic assays of the invention are described herein.

An agent for detecting NOV1, NOV2, NOV3, NOV4, FIZZX or MAMMX protein is an antibody capable of binding to NOV1, NOV2, NOV3, NOV4, FIZZX or MAMMX protein, preferably an antibody with a detectable label. Antibodies can be polyclonal, or more preferably, monoclonal. An intact antibody, or a fragment thereof (e.g., F_{ab} or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect NOV1, NOV2, NOV3, NOV4, FIZZX or MAMMX mRNA, protein, or genomic DNA in a biological sample in vitro as well as in vivo. For example, in vitro techniques for detection of NOV1, NOV2, NOV3, NOV4, FIZZX or MAMMX mRNA include Northern hybridizations and in situ hybridizations. In vitro techniques for detection of NOV1, NOV2, NOV3, NOV4, FIZZX or MAMMX protein include enzyme linked immunosorbent assays (ELISAs), Western blots, immunoprecipitations and immunofluorescence. In vitro techniques for detection of NOV1, NOV2, NOV3, NOV4, FIZZX or MAMMX genomic DNA include Southern hybridizations. Furthermore, in vivo techniques for detection of NOV1, NOV2, NOV-3, NOV4, FIZZX or MAMMX protein include introducing into a subject a labeled anti-NOV1, NOV2, NOV3, NOV4, FIZZX or MAMMX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOV1, NOV2, NOV3, NOV4, FIZZX or MAMMX protein, mRNA, or genomic DNA, such that the presence of NOV1, NOV2, NOV3, NOV4, FIZZX or MAMMX protein, mRNA or genomic DNA is detected in the biological sample, and comparing the presence of NOV1, NOV2, NOV3, NOV4, FIZZX or MAMMX protein, mRNA or genomic DNA in the control sample with the presence of NOV1, NOV2, NOV3, NOV4, FIZZX or MAMMX protein, mRNA or genomic DNA in the test sample.

The invention also encompasses kits for detecting the presence of NOV1, NOV2, NOV3, NOV4, FIZZX or MAMMX in a biological sample. For example, the kit can comprise: a labeled compound or agent capable of detecting NOV1, NOV2, NOV3, NOV4, FIZZX or MAMMX protein or mRNA in a biological sample; means for determining the amount of NOV1, NOV2, NOV3, NOV4, FIZZX or MAMMX in the sample; and means for comparing the amount of NOV1, NOV2, NOV3, NOV4, FIZZX or MAMMX in the sample with a standard. The compound or agent can be packaged in a suitable container. The kit can further comprise instructions for using the kit to detect NOV1, NOV2, NOV3, NOV4, FIZZX or MAMMX protein or nucleic acid.

Prognostic Assays

The diagnostic methods described herein can furthermore be utilized to identify subjects having or at risk of developing a disease or disorder associated with aberrant NOV1, NOV2, NOV3, NOV4, FIZZX or MAMMX expression or activity. For example, the assays described herein, such as the preceding diagnostic assays or the following assays, can be utilized to identify a subject having or at risk of developing a disorder associated with NOV1, NOV2, NOV3, NOV4, FIZZX or MAMMX protein, nucleic acid expression or activity such as cancer or fibrotic disorders. Alternatively, the prognostic assays can be utilized to identify a subject having or at risk for developing a disease or disorder. Thus, the present invention provides a method for identifying a disease or disorder associated with aberrant NOV1, NOV2, NOV3, NOV4, FIZZX or MAMMX expression or activity in which a test sample is obtained from a subject and NOV1, NOV2, NOV3, NOV4, FIZZX or MAMMX protein or nucleic acid (e.g., mRNA, genomic DNA) is detected, wherein the presence of NOV1, NOV2, NOV3, NOV4, FIZZX or MAMMX protein or nucleic acid is diagnostic for a subject having or at risk of developing a disease or disorder associated with aberrant NOV1, NOV2, NOV3, NOV4, FIZZX or MAMMX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOV1, NOV2, NOV3, NOV4, FIZZX or MAMMX expression or activity. For example, such methods can be used to determine whether a subject can be effectively treated with an agent for a disorder, such as cancer or preclampsia. Thus, the present invention provides methods for determining whether a subject can be effectively treated with an agent for a disorder associated with aberrant NOV1, NOV2, NOV3, NOV4, FIZZX or MAMMX expression or activity in which a test sample is obtained and NOV1, NOV2, NOV3, NOV4, FIZZX or MAMMX protein or nucleic acid is detected (e.g., wherein the presence of NOV1, NOV2, NOV3, NOV4, FIZZX or MAMMX protein or nucleic acid is diagnostic for a subject that can be administered the agent to treat a disorder associated with aberrant NOV1, NOV2, NOV3, NOV4, FIZZX or MAMMX expression or activity.)

The methods of the invention can also be used to detect genetic lesions in a NOV1, NOV2, NOV3, NOV4, FIZZX or MAMMX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOV1, NOV2, NOV3, NOV4, FIZZX or MAMMX -protein, or the mis-expression of the NOV1, NOV2, NOV3, NOV4, FIZZX or MAMMX gene. For example, such genetic lesions can be detected by ascertaining the existence of at least one of (1) a deletion of one or more nucleotides from a NOV1, NOV2, NOV3, NOV4, FIZZX or MAMMX gene; (2) an addition of one or more nucleotides to a NOV1, NOV2, NOV3, NOV4, FIZZX or MAMMX gene; (3) a substitution of one or more nucleotides of a NOV1, NOV2, NOV3, NOV4, FIZZX or MAMMX gene, (4) a chromosomal rearrangement of a NOV1, NOV2, NOV3, NOV4, FIZZX or MAMMX gene; (5) an alteration in the level of a messenger RNA transcript of a NOV1, NOV2, NOV3, NOV4, FIZZX or MAMMX gene, (6) aberrant modification of a NOV1, NOV2, NOV3, NOV4, FIZZX or MAMMX gene, such as of the methylation pattern of the genomic DNA, (7) the presence of a non-wild type splicing pattern of a messenger RNA transcript of a NOV1, NOV2, NOV3, NOV4, FIZZX or MAMMX gene, (8) a non-wild type level of a NOV1, NOV2, NOV3, NOV4, FIZZX or MAMMX -protein, (9) allelic loss of a NOV1, NOV2, NOV3, NOV4, FIZZX or MAMMX gene, and (10) inappropriate post-translational modification of a NOV1, NOV2, NOV3, NOV4, FIZZX or MAMMX -protein. As described herein, there are a large number of assay techniques known in the art which can be used for detecting lesions in a NOV1, NOV2, NOV3, NOV4, FIZZX or MAMMX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91 :360-364), the latter of which can be particularly useful for detecting point mutations in the NOV1, NOV2, NOV3, NOV4, FIZZX or MAMMX -gene (see Abravaya et al. (1995) Nucl Acids Res 23 :675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOV1, NOV2, NOV3, NOV4, FIZZX or MAMMX gene under conditions such that hybridization and amplification of the NOV1, NOV2, NOV3, NOV4, FIZZX or MAMMX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci USA 87:1874-1878), transcriptional amplification system (Kwoh, et al., 1989, Proc Natl Acad Sci USA 86:1173-1177), Q-Beta Replicase (Lizardi et al, 1988, BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOV1, NOV2, NOV3, NOV4, FIZZX or MAMMX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,493,531) can be used to score for the presence of specific mutations by development or loss of a ribozyme cleavage site.

In other embodiments, genetic mutations in NOV1, NOV2, NOV3, NOV4, FIZZX or MAMMX can be identified by hybridizing a sample and control nucleic acids, e.g., DNA or RNA, to high density arrays containing hundreds or thousands of oligonucleotides probes (Cronin et al. (1996) Human Mutation 7: 244-255; Kozal et al. (1996) Nature Medicine 2: 753-759). For example, genetic mutations in NOV1, NOV2, NOV3, NOV4, FIZZX or MAMMX can be identified in two dimensional arrays containing light-generated DNA probes as described in Cronin et al. above. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOV1, NOV2, NOV3, NOV4, FIZZX or MAMMX gene and detect mutations by comparing the sequence of the sample NOV1, NOV2, NOV3, NOV4, FIZZX or MAMMX with the corresponding wild-type (control) sequence. Examples of sequencing reactions include those based on techniques developed by Maxim and Gilbert (1977) PNAS 74:560 or Sanger (1977) PNAS 74:5463. It is also contemplated that any of a variety of automated sequencing procedures can be utilized when performing the diagnostic assays (Naeve et al., (1995) Biotechniques 19:448), including sequencing by mass spectrometry (see, e.g., PCT International Publ. No. WO 94/16101; Cohen et al. (1996) Adv Chromatogr 36:127-162; and Griffin et al. (1993) Appl Biochem Biotechnol 38:147-159).

Other methods for detecting mutations in the NOV1, NOV2, NOV3, NOV4, FIZZX or MAMMX gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type NOV1, NOV2, NOV3, NOV4, FIZZX or MAMMX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc Natl Acad Sci USA 85:4397; Saleeba et al (1992) Methods Enzymol 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOV1, NOV2, NOV3, NOV4, FIZZX or MAMMX cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a NOV1, NOV2, NOV3, NOV4, FIZZX or MAMMX sequence, e.g., a wild-type NOV1, NOV2, NOV3, NOV4, FIZZX or MAMMX sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in NOV1, NOV2, NOV3, NOV4, FIZZX or MAMMX genes. For example, single strand conformation polymorphism (SSCP) may be used to detect differences in electrophoretic mobility between mutant and wild type nucleic acids (Orita et al. (1989) Proc Natl Acad Sci USA: 86:2766, see also Cotton (1993) Mutat Res 285:125-144; Hayashi (1992) Genet Anal Tech Appl 9:73-79). Single-stranded DNA fragments of sample and control NOV1, NOV2, NOV3, NOV4, FIZZX or MAMMX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol Cell Probes 6: 1). It is anticipated that in certain embodiments amplification may also be performed using Taq ligase for amplification (Barany (1991) Proc Natl Acad Sci USA 88:189). In such cases, ligation will occur only if there is a perfect match at the 3′ end of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOV1, NOV2, NOV3, NOV4, FIZZX or MAMMX gene.

Furthermore, any cell type or tissue, preferably peripheral blood leukocytes, in which NOV1, NOV2, NOV3, NOV4, FIZZX or MAMMX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOV1, NOV2, NOV3, NOV4, FIZZX or MAMMX activity (e.g., NOV1, NOV2, NOV3, NOV4, FIZZX or MAMMX gene expression), as identified by a screening assay described herein can be administered to individuals to treat (prophylactically or therapeutically) disorders (e.g., cancer or gestational disorders) associated with aberrant NOV1, NOV2, NOV3, NOV4, FIZZX or MAMMX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOV1, NOV2, NOV3, NOV4, FIZZX or MAMMX protein, expression of NOV1, NOV2, NOV3, NOV4, FIZZX or MAMMX nucleic acid, or mutation content of NOV1, NOV2, NOV3, NOV4, FIZZX or MAMMX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, Clin Exp Pharmacol Physiol, 1996, 23:983-985 and Linder, Clin Chem, 1997, 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of NOV1, NOV2, NOV3, NOV4, FIZZX or MAMMX protein, expression of NOV1, NOV2, NOV3, NOV4, FIZZX or MAMMX nucleic acid, or mutation content of NOV1, NOV2, NOV3, NOV4, FIZZX or MAMMX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOV1, NOV2, NOV3, NOV4, FIZZX or MAMMX modulator, such as a modulator identified by one of the exemplary screening assays described herein.

Monitoring of Effects During Clinical Trials

Monitoring the influence of agents (e.g., drugs, compounds) on the expression or activity of NOV1, NOV2, NOV3, NOV4, FIZZX or MAMMX (e.g., the ability to modulate aberrant cell proliferation and/or differentiation) can be applied not only in basic drug screening, but also in clinical trials. For example, the effectiveness of an agent determined by a screening assay as described herein to increase NOV1, NOV2, NOV3, NOV4, FIZZX or MAMMX gene expression, protein levels, or upregulate NOV1, NOV2, NOV3, NOV4, FIZZX or MAMMX activity, can be monitored in clinical trails of subjects exhibiting decreased NOV1, NOV2, NOV3, NOV4, FIZZX or MAMMX gene expression, protein levels, or downregulated NOV1, NOV2, NOV3, NOV4, FIZZX or MAMMX activity. Alternatively, the effectiveness of an agent determined by a screening assay to decrease NOV1, NOV2, NOV3, NOV4, FIZZX or MAMMX gene expression, protein levels, or downregulate NOV1, NOV2, NOV3, NOV4, FIZZX or MAMMX activity, can be monitored in clinical trails of subjects exhibiting increased NOV1, NOV2, NOV3, NOV4, FIZZX or MAMMX gene expression, protein levels, or upregulated NOV1, NOV2, NOV3, NOV4, FIZZX or MAMMX activity. In such clinical trials, the expression or activity of NOV1, NOV2, NOV3, NOV4, FIZZX or MAMMX and, preferably, other genes that have been implicated in, for example, a cellular proliferation disorder can be used as a “read out” or markers of the immune responsiveness of a particular cell.

For example, and not by way of limitation, genes, including NOV1, NOV2, NOV3, NOV4, FIZZX or MAMMX, that are modulated in cells by treatment with an agent (e.g., compound, drug or small molecule) that modulates NOV1, NOV2, NOV3, NOV4, FIZZX or MAMMX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOV1, NOV2, NOV3, NOV4, FIZZX or MAMMX and other genes implicated in the disorder. The levels of gene expression (i. e., a gene expression pattern) can be quantified by Northern blot analysis or RT-PCR, as described herein, or alternatively by measuring the amount of protein produced, by one of the methods as described herein, or by measuring the levels of activity of NOV1, NOV2, NOV3, NOV4, FIZZX or MAMMX or other genes. In this way, the gene expression pattern can serve as a marker, indicative of the physiological response of the cells to the agent. Accordingly, this response state may be determined before, and at various points during, treatment of the individual with the agent.

In one embodiment, the present invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOV1, NOV2, NOV3, NOV4, FIZZX or MAMMX protein, mRNA, or genomic DNA in the preadministration sample; (iii) obtaining one or more post-administration samples from the subject; (iv) detecting the level of expression or activity of the NOV1, NOV2, NOV3, NOV4, FIZZX or MAMMX protein, mRNA, or genomic DNA in the post-administration samples; (v) comparing the level of expression or activity of the NOV1, NOV2, NOV3, NOV4, FIZZX or MAMMX protein, mRNA, or genomic DNA in the pre-administration sample with the NOV1, NOV2, NOV3, NOV4, FIZZX or MAMMX protein, mRNA, or genomic DNA in the post administration sample or samples; and (vi) altering the administration of the agent to the subject accordingly. For example, increased administration of the agent may be desirable to increase the expression or activity of NOV1, NOV2, NOV3, NOV4, FIZZX or MAMMX to higher levels than detected, i.e., to increase the effectiveness of the agent. Alternatively, decreased administration of the agent may be desirable to decrease expression or activity of NOV1, NOV2, NOV3, NOV4, FIZZX or MAMMX to lower levels than detected, i.e., to decrease the effectiveness of the agent.

Methods of Treatment

The present invention provides for both prophylactic and therapeutic methods of treating a subject at risk of (or susceptible to) a disorder or having a disorder associated with aberrant NOV1, NOV2, NOV3, NOV4, FIZZX or MAMMX expression or activity. Methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to, (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic Methods

In one aspect, the invention provides a method for preventing, in a subject, a disease or condition associated with an aberrant NOV1, NOV2, NOV3, NOV4, FIZZX or MAMMX expression or activity, by administering to the subject an agent that modulates NOV1, NOV2, NOV3, NOV4, FIZZX or MAMMX expression or at least one NOV1, NOV2, NOV3, NOV4, FIZZX or MAMMX activity. Subjects at risk for a disease that is caused or contributed to by aberrant NOV1, NOV2, NOV3, NOV4, FIZZX or MAMMX expression or activity can be identified by, for example, any or a combination of diagnostic or prognostic assays as described herein. Administration of a prophylactic agent can occur prior to the manifestation of symptoms characteristic of the NOV1, NOV2, NOV3, NOV4, FIZZX or MAMMX aberrancy, such that a disease or disorder is prevented or, alternatively, delayed in its progression. Depending on the type of NOV1, NOV2, NOV3, NOV4, FIZZX or MAMMX aberrancy, for example, a NOV1, NOV2, NOV3, NOV4, FIZZX or MAMMX agonist or NOV1, NOV2, NOV3, NOV4, FIZZX or MAMMX antagonist agent can be used for treating the subject. The appropriate agent can be determined based on screening assays described herein. The prophylactic methods of the present invention are further discussed in the following subsections.

Therapeutic Methods

Another aspect of the invention pertains to methods of modulating NOV1, NOV2, NOV3, NOV4, FIZZX or MAMMX expression or activity for therapeutic purposes. The modulatory method of the invention involves contacting a cell with an agent that modulates one or more of the activities of NOV1, NOV2, NOV3, NOV4, FIZZX or MAMMX protein activity associated with the cell. An agent that modulates NOV1, NOV2, NOV3, NOV4, FIZZX or MAMMX protein activity can be an agent as described herein, such as a nucleic acid or a protein, a naturally-occurring cognate ligand of a NOV1, NOV2, NOV3, NOV4, FIZZX or MAMMX protein, a peptide, a NOV1, NOV2, NOV3, NOV4, FIZZX or MAMMX peptidomimetic, or other small molecule. In one embodiment, the agent stimulates one or more NOV1, NOV2, NOV3, NOV4, FIZZX or MAMMX protein activity. Examples of such stimulatory agents include active NOV1, NOV2, NOV3, NOV4, FIZZX or MAMMX protein and a nucleic acid molecule encoding NOV1, NOV2, NOV3, NOV4, FIZZX or MAMMX that has been introduced into the cell. In another embodiment, the agent inhibits one or more NOV1, NOV2, NOV3, NOV4, FIZZX or MAMMX protein activity. Examples of such inhibitory agents include antisense NOV1, NOV2, NOV3, NOV4, FIZZX or MAMMX nucleic acid molecules and anti-NOV1, NOV2, NOV3, NOV4, FIZZX or MAMMX antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a NOV1, NOV2, NOV3, NOV4, FIZZX or MAMMX protein or nucleic acid molecule. In one embodiment, the method involves administering an agent (e.g., an agent identified by a screening assay described herein), or combination of agents that modulates (e.g., upregulates or downregulates) NOV1, NOV2, NOV3, NOV4, FIZZX or MAMMX expression or activity. In another embodiment, the method involves administering a NOV1, NOV2, NOV3, NOV4, FIZZX or MAMMX protein or nucleic acid molecule as therapy to compensate for reduced or aberrant NOV1, NOV2, NOV3, NOV4, FIZZX or MAMMX expression or activity.

Stimulation of NOV1, NOV2, NOV3, NOV4, FIZZX or MAMMX activity is desirable in situations in which NOV1, NOV2, NOV3, NOV4, FIZZX or MAMMX is abnormally downregulated and/or in which increased NOV1, NOV2, NOV3, NOV4, FIZZX or MAMMX activity is likely to have a beneficial effect. One example of such a situation is where a subject has a disorder characterized by aberrant cell proliferation and/or differentiation (e.g., cancer). Another example of such a situation is where the subject has a gestational disease (e.g., preclampsia).

Malignancies

An aforementioned protein or polynucleotide of the invention may be involved in the regulation of cell proliferation. Accordingly, Therapeutics of the present invention may be useful in the therapeutic or prophylactic treatment of diseases or disorders that are associated with cell hyperproliferation and/or loss of control of cell proliferation (e.g., cancers, malignancies and tumors). For a review of such hyperproliferation disorders, see e.g., Fishman, et al., 1985. MEDICINE, 2nd ed., J. B. Lippincott Co., Philadelphia, Pa.

Therapeutics of the present invention may be assayed by any method known within the art for efficacy in treating or preventing malignancies and related disorders. Such assays include, but are not limited to, in vitro assays utilizing transformed cells or cells derived from the patient's tumor, as well as in vivo assays using animal models of cancer or malignancies. Potentially effective Therapeutics are those that, for example, inhibit the proliferation of tumor-derived or transformed cells in culture or cause a regression of tumors in animal models, in comparison to the controls.

In the practice of the present invention, once a malignancy or cancer has been shown to be amenable to treatment by modulating (i.e., inhibiting, antagonizing or agonizing) activity, that cancer or malignancy may subsequently be treated or prevented by the administration of a Therapeutic that serves to modulate protein function.

Premalignant conditions

The Therapeutics of the present invention that are effective in the therapeutic or prophylactic treatment of cancer or malignancies may also be administered for the treatment of pre-malignant conditions and/or to prevent the progression of a pre-malignancy to a neoplastic or malignant state. Such prophylactic or therapeutic use is indicated in conditions known or suspected of preceding progression to neoplasia or cancer, in particular, where non-neoplastic cell growth consisting of hyperplasia, metaplasia or, most particularly, dysplasia has occurred. For a review of such abnormal cell growth see e.g., Robbins & Angell, 1976. BASIC PATHOLOGY, 2nd ed., W. B. Saunders Co., Philadelphia, Pa.

Hyperplasia is a form of controlled cell proliferation involving an increase in cell number in a tissue or organ, without significant alteration in its structure or function. For example, it has been demonstrated that endometrial hyperplasia often precedes endometrial cancer. Metaplasia is a form of controlled cell growth in which one type of mature or fully differentiated cell substitutes for another type of mature cell. Metaplasia may occur in epithelial or connective tissue cells. Dysplasia is generally considered a precursor of cancer, and is found mainly in the epithelia. Dysplasia is the most disorderly form of non-neoplastic cell growth, and involves a loss in individual cell uniformity and in the architectural orientation of cells. Dysplasia characteristically occurs where there exists chronic irritation or inflammation, and is often found in the cervix, respiratory passages, oral cavity, and gall bladder.

Alternatively, or in addition to the presence of abnormal cell growth characterized as hyperplasia, metaplasia, or dysplasia, the presence of one or more characteristics of a transformed or malignant phenotype displayed either in vivo or in vitro within a cell sample derived from a patient, is indicative of the desirability of prophylactic/therapeutic administration of a Therapeutic that possesses the ability to modulate activity of An aforementioned protein. Characteristics of a transformed phenotype include, but are not limited to: (i) morphological changes; (ii) looser substratum attachment; (iii) loss of cell-to-cell contact inhibition; (iv) loss of anchorage dependence; (v) protease release; (vi) increased sugar transport; (vii) decreased serum requirement; (viii) expression of fetal antigens, (ix) disappearance of the 250 kDal cell-surface protein, and the like. See e.g., Richards, et al., 1986. MOLECULAR PATHOLOGY, W. B. Saunders Co., Philadelphia, Pa.

In a specific embodiment of the present invention, a patient that exhibits one or more of the following predisposing factors for malignancy is treated by administration of an effective amount of a Therapeutic: (i) a chromosomal translocation associated with a malignancy (e.g., the Philadelphia chromosome (bcr/abl for chronic myelogenous leukemia and t(14;18) for follicular lymphoma, etc.); (ii) familial polyposis or Gardner's syndrome (possible forerunners of colon cancer); (iii) monoclonal gammopathy of undetermined significance (a possible precursor of multiple myeloma) and (iv) a first degree kinship with persons having a cancer or pre-cancerous disease showing a Mendelian (genetic) inheritance pattern (e.g., familial polyposis of the colon, Gardner's syndrome, hereditary exostosis, polyendocrine adenomatosis, Peutz-Jeghers syndrome, neurofibromatosis of Von Recklinghausen, medullary thyroid carcinoma with amyloid production and pheochromocytoma, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia and Bloom's syndrome).

In another embodiment, a Therapeutic of the present invention is administered to a human patient to prevent the progression to breast, colon, lung, pancreatic, or uterine cancer, or melanoma or sarcoma.

Hyperproliferative and dysproliferative disorders

In one embodiment of the present invention, a Therapeutic is administered in the therapeutic or prophylactic treatment of hyperproliferative or benign dysproliferative disorders. The efficacy in treating or preventing hyperproliferative diseases or disorders of a Therapeutic of the present invention may be assayed by any method known within the art. Such assays include in vitro cell proliferation assays, in vitro or in vivo assays using animal models of hyperproliferative diseases or disorders, or the like. Potentially effective Therapeutics may, for example, promote cell proliferation in culture or cause growth or cell proliferation in animal models in comparison to controls.

Specific embodiments of the present invention are directed to the treatment or prevention of cirrhosis of the liver (a condition in which scarring has overtaken normal liver regeneration processes); treatment of keloid (hypertrophic scar) formation causing disfiguring of the skin in which the scarring process interferes with normal renewal; psoriasis (a common skin condition characterized by excessive proliferation of the skin and delay in proper cell fate determination); benign tumors; fibrocystic conditions and tissue hypertrophy (e.g., benign prostatic hypertrophy).

Neurodegenerative disorders

NOV1, NOV2, NOV3, NOV4, FIZZX or MAMMX protein may be implicated in the deregulation of cellular maturation and apoptosis, which are both characteristic of neurodegenerative disease. Accordingly, Therapeutics of the invention, particularly but not limited to those that modulate (or supply) activity of an aforementioned protein, may be effective in treating or preventing neurodegenerative disease. Therapeutics of the present invention that modulate the activity of an aforementioned protein involved in neurodegenerative disorders can be assayed by any method known in the art for efficacy in treating or preventing such neurodegenerative diseases and disorders. Such assays include in vitro assays for regulated cell maturation or inhibition of apoptosis or in vivo assays using animal models of neurodegenerative diseases or disorders, or any of the assays described below. Potentially effective Therapeutics, for example but not by way of limitation, promote regulated cell maturation and prevent cell apoptosis in culture, or reduce neurodegeneration in animal models in comparison to controls.

Once a neurodegenerative disease or disorder has been shown to be amenable to treatment by modulation activity, that neurodegenerative disease or disorder can be treated or prevented by administration of a Therapeutic that modulates activity. Such diseases include all degenerative disorders involved with aging, especially osteoarthritis and neurodegenerative disorders.

Disorders related to organ transplantation

NOV1, NOV2, NOV3, NOV4, FIZZX or MAMMX may be implicated in disorders related to organ transplantation, in particular but not limited to organ rejection. Therapeutics of the invention, particularly those that modulate (or supply) activity, may be effective in treating or preventing diseases or disorders related to organ transplantation. Therapeutics of the invention (particularly Therapeutics that modulate the levels or activity of an aforementioned protein) can be assayed by any method known in the art for efficacy in treating or preventing such diseases and disorders related to organ transplantation. Such assays include in vitro assays for using cell culture models as described below, or in vivo assays using animal models of diseases and disorders related to organ transplantation, see e.g., below. Potentially effective Therapeutics, for example but not by way of limitation, reduce immune rejection responses in animal models in comparison to controls.

Accordingly, once diseases and disorders related to organ transplantation are shown to be amenable to treatment by modulation of activity, such diseases or disorders can be treated or prevented by administration of a Therapeutic that modulates activity.

Cardiovascular Disease

NOV1, NOV2, NOV3, NOV4, FIZZX or MAMMX may be implicated in cardiovascular disorders, including in atherosclerotic plaque formation. Diseases such as cardiovascular disease, including cerebral thrombosis or hemorrhage, ischemic heart or renal disease, peripheral vascular disease, or thrombosis of other major vessel, and other diseases, including diabetes mellitus, hypertension, hypothyroidism, cholesterol ester storage disease, systemic lupus erythematosus, homocysteinemia, and familial protein or lipid processing diseases, and the like, are either directly or indirectly associated with atherosclerosis. Accordingly, Therapeutics of the invention, particularly those that modulate (or supply) activity or formation may be effective in treating or preventing atherosclerosis-associated diseases or disorders. Therapeutics of the invention (particularly Therapeutics that modulate the levels or activity) can be assayed by any method known in the art, including those described below, for efficacy in treating or preventing such diseases and disorders.

A vast array of animal and cell culture models exist for processes involved in atherosclerosis. A limited and non-exclusive list of animal models includes knockout mice for premature atherosclerosis (Kurabayashi and Yazaki, 1996, Int. Angiol. 15: 187-194), transgenic mouse models of atherosclerosis (Kappel et al., 1994, FASEB J. 8: 583-592), antisense oligonucleotide treatment of animal models (Callow, 1995, Curr. Opin. Cardiol. 10: 569-576), transgenic rabbit models for atherosclerosis (Taylor, 1997, Ann. N.Y. Acad. Sci 811: 146-152), hypercholesterolemic animal models (Rosenfeld, 1996, Diabetes Res. Clin. Pract. 30 Suppl.: 1-11), hyperlipidemic mice (Paigen et al., 1994, Curr. Opin. Lipidol. 5: 258-264), and inhibition of lipoxygenase in animals (Sigal et al., 1994, Ann. N.Y. Acad. Sci. 714: 211-224). In addition, in vitro cell models include but are not limited to monocytes exposed to low density lipoprotein (Frostegard et al., 1996, Atherosclerosis 121: 93-103), cloned vascular smooth muscle cells (Suttles et al., 1995, Exp. Cell Res. 218: 331-338), endothelial cell-derived chemoattractant exposed T cells (Katz et al., 1994, J. Leukoc. Biol. 55: 567-573), cultured human aortic endothelial cells (Farber et al., 1992, Am. J. Physiol. 262: H1088-1085), and foam cell cultures (Libby et al., 1996, Curr Opin Lipidol 7: 330-335). Potentially effective Therapeutics, for example but not by way of limitation, reduce foam cell formation in cell culture models, or reduce atherosclerotic plaque formation in hypercholesterolemic mouse models of atherosclerosis in comparison to controls.

Accordingly, once an atherosclerosis-associated disease or disorder has been shown to be amenable to treatment by modulation of activity or formation, that disease or disorder can be treated or prevented by administration of a Therapeutic that modulates activity.

Cytokine and Cell Proliferation/Differentiation Activity

A NOV1, NOV2, NOV3, NOV4, FIZZX or MAMMX protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK.

The activity of a protein of the invention may, among other means, be measured by the following methods: Assays for T-cell or thymocyte proliferation include without limitation those described in: CURRENT PROTOCOLS IN IMMUNOLOGY, Ed by Coligan et al., Greene Publishing Associates and Wiley-Interscience (Chapter 3 and Chapter 7); Takai et al., J Immunol 137:3494-3500, 1986; Bertagnolli et al., J Immunol 145:1706-1712, 1990; Bertagnolli et al., Cell Immunol 133:327-341, 1991; Bertagnolli, et al., J Immunol 149:3778-3783, 1992; Bowman et al., J Immunol 152:1756-1761, 1994.

Assays for cytokine production and/or proliferation of spleen cells, lymph node cells or thymocytes include, without limitation, those described by Kruisbeek and Shevach, In: CURRENT PROTOCOLS IN IMMUNOLOGY. Coligan et al., eds. Vol 1, pp. 3.12.1-14, John Wiley and Sons, Toronto 1994; and by Schreiber, In: CURRENT PROTOCOLS IN IMMUNOLOGY. Coligan eds. Vol. 1 pp. 6.8.1-8, John Wiley and Sons, Toronto 1994.

Assays for proliferation and differentiation of hematopoietic and lymphopoietic cells include, without limitation, those described by Bottomly et al., In: CURRENT PROTOCOLS IN IMMUNOLOGY. Coligan et al., eds. Vol 1 pp. 6.3.1-6.3.12, John Wiley and Sons, Toronto 1991; deVries et al., J Exp Med 173:1205-1211, 1991; Moreau et al., Nature 336:690-692, 1988; Greenberger et al., Proc Natl Acad Sci U.S.A. 80:2931-2938, 1983; Nordan, In: CURRENT PROTOCOLS IN IMMUNOLOGY. Coligan et al., eds. Vol 1 pp. 6.6.1-5, John Wiley and Sons, Toronto 1991; Smith et al., Proc Natl Acad Sci U.S.A. 83:1857-1861, 1986; Measurement of human Interleukin 11-Bennett, et al. In: CURRENT PROTOCOLS IN IMMUNOLOGY. Coligan et al., eds. Vol 1 pp. 6.15.1 John Wiley and Sons, Toronto 1991; Ciarletta, et al., In: CURRENT PROTOCOLS IN IMMUNOLOGY. Coligan et al., eds. Vol 1 pp. 6.13.1, John Wiley and Sons, Toronto 1991.

Assays for T-cell clone responses to antigens (which will identify, among others, proteins that affect APC-T cell interactions as well as direct T-cell effects by measuring proliferation and cytokine production) include, without limitation, those described In: CURRENT PROTOCOLS IN IMMUNOLOGY. Coligan et al., eds., Greene Publishing Associates and Wiley-Interscience (Chapter 3Chapter 6, Chapter 7); Weinberger et al., Proc Natl Acad Sci USA 77:6091-6095, 1980; Weinberger et al., Eur J Immun 11:405-411, 1981; Takai et al., J Immunol 137:3494-3500, 1986; Takai et al., J Immunol 140:508-512, 1988.

Immune Stimulating or Suppressing Activity

A NOV1, NOV2, NOV3, NOV4, FIZZX or MAMMX protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by vital (e.g., HIV) as well as bacterial or fungal infections, or may result from autoimmune disorders. More specifically, infectious diseases causes by vital., bacterial., fungal or other infection may be treatable using a protein of the present invention, including infections by HIV, hepatitis viruses, herpesviruses, mycobacteria, Leishmania species., malaria species. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitus, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or energy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

Down regulating or preventing one or more antigen functions (including without limitation B lymphocyte antigen functions (such as, for example, B7), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to energize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc Natl Acad Sci USA, 89:11102-11105 (1992). In addition, murine models of GVHD (see Paul ed., FUNDAMENTAL IMMUNOLOGY, 25 Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and auto-antibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of auto-antibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythematosis in MRL/lpr/lpr mice or NZB hybrid mice, murine autoimmune collagen arthritis, diabetes mellitus in NOD mice and BB rats, and murine experimental myasthenia gravis (see Paul ed., FUNDAMENTAL IMMUNOLOGY, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic vital diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-vital immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I a chain protein and b ₂ microglobulin protein or an MHC class II a chain protein and an MHC class II b chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods: Suitable assays for thymocyte or splenocyte cytotoxicity include, without limitation, those described In: CURRENT PROTOCOLS IN IMMUNOLOGY. Coligan et al., eds. Greene Publishing Associates and Wiley-Interscience (Chapter 3, Chapter 7); Herrmann et al., Proc Natl Acad Sci USA 78:2488-2492, 1981; Herrmann et al., J Immunol 128:1968-1974, 1982; Handa et al., J Immunol 135:1564-1572, 1985; Takai et al., J Immunol 137:3494-3500, 1986; Takai et al., J Immunol 140:508-512, 1988; Herrmann et al., Proc Natl Acad Sci USA 78:2488-2492, 1981; Herrmann et al., J Immunol 128:1968-1974, 1982; Handa et al., J Immunol 135:1564-1572, 1985; Takai et al., J Immunol 137:3494-3500, 1986; Bowman et al., J Virology 61:1992-1998; Takai et al., J Immunol 140:508-512, 1988; Bertagnolli et al., Cell Immunol 133:327-341, 1991; Brown et al., J Immunol 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J Immunol 144:3028-3033, 1990; and Mond and Brunswick In: CURRENT PROTOCOLS IN IMMUNOLOGY. Coligan et al., eds. Vol 1 pp. 3.8.1-3.8.16, John Wiley and Sons, Toronto 1994.

Mixed lymphocyte reaction (MLR) assays (which will identify, among others, proteins that generate predominantly Th1 and CTL responses) include, without limitation, those described In: CURRENT PROTOCOLS IN IMMUNOLOGY. Coligan et al., eds. Greene Publishing Associates and Wiley-Interscience (Chapter 3, Chapter 7); Takai et al., J Immunol 137:3494-3500, 1986; Takai et al., J Immunol 140:508-512, 1988; Bertagnolli et al., J Immunol 149:3778-3783, 1992.

Dendritic cell-dependent assays (which will identify, among others, proteins expressed by dendritic cells that activate naive T-cells) include, without limitation, those described in: Guery et al., J Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J Virol 67:4062-4069, 1993; Huang et al., Science 264:961-965, 1994; Macatonia et al., J Exp Med 169:1255-1264, 1989; Bhardwaj et al., J Clin Investig 94:797-807, 1994; and Inaba et al., J Exp Med 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Res 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., Internat J Oncol 1:639-648, 1992.

Assays for proteins that influence early steps of T-cell commitment and development include, without limitation, those described in: Antica et al., Blood 84:111-117, 1994; Fine et al., Cell Immunol 155: 111-122, 1994; Galy et al., Blood 85:2770-2778, 1995; Toki et al., Proc Nat Acad Sci USA 88:7548-7551, 1991.

Hematopoiesis Regulating Activity

A NOV1, NOV2, NOV3, NOV4, FIZZX or MAMMX protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in vivo or ex vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by suitable assays. Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cell Biol 15:141-151, 1995; Keller et al., Mol Cell Biol 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In CULTURE OF HEMATOPOIETIC CELLS. Freshney, et al. eds. Vol pp. 265-268, Wiley-Liss, Inc., New York, N.Y 1994; Hirayama et al., Proc Natl Acad Sci USA 89:5907-5911, 1992; McNiece and Briddeli. In CULTURE OF HEMATOPOIETIC CELLS. Freshney, et al. eds. Vol pp. 23-39, Wiley-Liss, Inc., New York, N.Y. 1994; Neben et al., Exp Hematol 22:353-359, 1994; Ploemacher In CULTURE OF HEMATOPOIETIC CELLS. Freshney, et al. eds. Vol pp. 1-21, Wiley-Liss, Inc., New York, N.Y. 1994; Spoonceret al., In CULTURE OF HEMATOPOIETIC CELLS. Freshhey, et al. eds. Vol pp. 163-179, Wiley-Liss, Inc., New York, N.Y. 1994; Sutherland, In CULTURE OF HEMATOPOIETIC CELLS. Freshney, et al. eds. Vol pp. 139-162, Wiley-Liss, Inc., New York, N.Y. 1994.

Tissue Growth Activity

A NOV1, NOV2, NOV3, NOV4, FIZZX or MAMMX protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital., trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital., trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendonitis, carpal tunnel syndrome and other tendon or ligament defects. The compositions may also include an appropriate matrix and/or sequestering agent as a career as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing, pp. 71-112 (Maibach, H I and Rovee, D T, eds.), Year Book Medical Publishers, Inc., Chicago, as modified by Eaglstein and Menz, J. Invest. Dermatol 71:382-84 (1978).

Activin/Inhibin Activity

A NOV1, NOV2, NOV3, NOV4, FIZZX or MAMMX protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-b group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc Natl Acad Sci USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by following methods. Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: CURRENT PROTOCOLS IN IMMUNOLOGY, Coligan et al., eds. (Chapter 6.12, Measurement of alpha and beta Chemokines 6.12.1-6.12.28); Taub et al J Clin Invest 95:1370-1376, 1995; Lind et al. APMIS 103:140-146, 1995; Muller et al Eur J Immunol 25: 1744-1748; Gruberet al J Immunol 152:5860-5867, 1994; Johnston et al. J Immunol 153: 1762-1768, 1994.

Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods. Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Receptor/Ligand Activity

A protein of the present invention may also demonstrate activity as a receptor, receptor ligand or inhibitor or agonist of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selectins, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods. Suitable assays for receptor-ligand activity include without limitation those described in: CURRENT PROTOCOLS IN IMMUNOLOGY, Ed by Coligan et al., Greene Publishing Associates and Wiley-Interscience (Chapter 7.28, Measurement of Cellular Adhesion under static conditions 7.28.1-7.28.22), Takai et al., Proc Natl Acad Sci USA 84:6864-6868, 1987; Bierer et al., J. Exp. Med. 168:1145-1156, 1988; Rosenstein et al., J. Exp. Med. 169:149-160 1989; Stoltenborg et al., J Immunol Methods 175:59-68, 1994; Stitt et al., Cell 80:661-670, 1995.

Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

This invention is further illustrated by the following examples, which should not be construed as limiting. The contents of all references, patents and published patent applications cited throughout this application are hereby incorporated by reference.

### EXAMPLES

### Example 1

Expression of Clone FIZZX in Mammalian and Insect Cells

1.1. Cloning of FIZZX cDNA for mammalian and insect cell expression.

Based on the predicted reading frame, PCR primers were designed to amplify the coding region for hFIZZX. The forward primer was 5′-CGAGATCTCCACCATGAAAGCTCTCTGTCTCCTCCTCCTCCCTGCTCTGGGGCTGTTGGTGTCTAG (SEQ ID NO: 28), and the reverse primer was 5′-ATCTCGAGGGGCTGCACACGACAGCAGCGCGCTCCGGTCCAGTCCAT (SEQ ID NO: 29). PCR was initiated by heating 25 μl Mix 1 (75 pmoles primers, 4 μg adult bone marrow cDNA, 5 μmoles dNTPs) and 25 μl Mix 2 [1 unit Fidelity Expand polymerase (Boehringer Mannheim), 5 μl 10× Fidelity Expand Buffer] separately at 96° C. for 20 seconds. Mixes 1 and 2 were then pooled, and the following PCR cycling parameters were used: 96° C., 3 min (1 cycle); 96° C., 30 sec, 55° C., 1 min, 68° C., 2 min (10 cycles); 96° C., 30 sec, 60° C., 1 min, 68° C., 2 min (20 cycles); 72° C., 7 min (1 cycle). After PCR, a single DNA fragment of approximately 0.4 kb was obtained. The DNA fragment was digested with BglII and XhoI restriction enzymes, and cloned into the pcDNA3.1 V5His vector (Invitrogen, Carlsbad, Calif.) or into the pBIgHis vector (CuraGen Corporation). The FIZZX insert was verified by DNA sequence analysis. The resulting expression vectors are called pcDNA3.1V5HisFIZZX for mammalian kidney 293 cell expression and pBIgHisFIZZX for insect cell expression.

1.2. Expression of hFIZZX in human embryonic kidney 293 cells.

The pcDNA3.1V5HisFIZZX vector was transfected into 293 cells using the LipofectaminePlus reagent following the manufacturer's instructions (Gibco/BRL). The cell pellet and supernatant were harvested 72 hours after transfection and examined for hFIZZX expression by Western blotting (reducing conditions) with an anti-V5 antibody. The hFIZZX protein was expressed as a 20-kDa protein secreted by 293 cells.

1.3. Construction of pBIgHis baculo expression vector.

To construct the pBIgHis expression vector, we designed oligonucleotide primers to amplify the Fc fragment of the human immunoglobulin heavy chain. The forward primer was 5′-CCGCTCGAGTGAGCCCAAATCTTGTGACAAA (SEQ ID NO: 30) and the reverse primer was 5′-GCTCTAGACTTTTACCCGGGGACAGGGAG (SEQ ID NO: 31). PCR was initiated by heating 25 ul Mix 1 (75 pmoles primers, 4 ug adult testis cDNA, 5 umoles dNTPs) and 25 ul Mix 2 [1 unit Fidelity Expand polymerase (Boehringer Mannheim), 5 μl 10× Fidelity Expand Buffer] separately at 96° C. for 20 seconds. Mixes 1 and 2 were then pooled, and the following PCR cycling parameters were used: 96° C., 3 min (1 cycle); 96° C., 30 sec, 55° C., 1 min, 68° C., 2 min (10 cycles); 96° C., 30 sec, 60° C., 1 min, 68° C., 2 min (20 cycles); 72° C., 7 min (1 cycle). After PCR, a single DNA fragment of approximately 0.75 kb was obtained. The DNA fragment was digested with XhoI and XbaI restriction enzymes and cloned into the pCDNA3.1V5His(B) expression vector (Invitrogen, Carlsbad, Calif.). This vector is named as pCDNA3.1 Ig and contains Fc fragment fused to V5 epitope and 6×His tag. To introduce a recombinant TEV protease cleavage site adjacent to the N-terminus of the Fc fragment, the inventors designed two oligonucleotides (SEQ ID NO:32:5′-AATTCTGCAGCGAAAACCTGTATTTTCAGGGT and SEQ ID NO:33:5′-TCGAACCCTGAAAATACAGGTTTTCGCTGCAG) and purified the annealed oligos on a 20% polyacrylamide gel. The double stranded oligo DNA was then ligated into pCNA3.1 Ig digested with EcoRI and XhoI. The resulting plasmid was digested with PstI and PmeI to release a DNA fragment of approximately 0.9 kb, which was ligated into pBlueBac4.5 digested with PstI and Smal (Invitrogen, Carlsbad, Calif.). The plasmid construct obtained is named as pBIgHis. The Fc fragment was verified by sequence analysis.

1.4. Construction and isolation of recombinant cells expressing hFIZZX.

pBIgHisFIZZX plasmid DNA was co-transfected with linearized baculovirus DNA (Bac-N-Blue) into SF9 insect cells using liposome-mediated transfer as described by the manufacturer (Invitrogen). Briefly, transfection mixtures containing 4 ug of pBIgHisFIZZX, 0.5 ug of Bac-N-Blue™ and InsectinPlus™ liposomes were added to 60 mm culture dishes seeded with 2×10⁶ SF9 cells, and incubated with rocking at 27° C. for 4 hours. Fresh culture medium was added and cultures were further incubated for 4 days. The culture medium was then harvested and recombinant viruses were isolated using standard plaque purification procedures. Recombinant viruses expressing b-galactosidase as a marker were readily distinguished from non-recombinant viruses by visually inspecting agarose overlays for blue plaques. Viral stocks were generated by propagation on SF9 cells and screened for expression of hFIZZX protein by SDS-PAGE and Western blot analyses (reducing conditions, anti-V5 antibody). The hFIZZX protein is secreted as a 45-kDa protein, corresponding to the fusion of FIZZX with the Ig F_{c} sequence.

Clone FIZZX cDNA was also subcloned into the pcDNA3 mammalian expression factor and assayed for transforming activity following transfection into murine fibroblasts (NIH 3T3). No foci were generated by the clone FIZZX cDNA, indicating that it is non-transforming in this system.

Using the same construct, clone 2155657 was transiently transfected into human 293 kidney epithelial cells. The supernatant from these cells was found to contain FIZZX protein and was assayed for the ability to activate immediate early response genes (EGR-1, ATF-3, FOS, MKP-1, c-JUN, JUNB) by a TaqMan assay. No immediate early response gene activation was found in cells treated with FIZZX.

### Example 2

Northern Analysis OF CLONE NOV3

2.1. Probe Production.

A NOV3 gene fragment (nucleotides 40-606) cloned into pCR2.1 (Invitrogen) was used as a template in a PCR reaction. The primers (SEQ ID NO: 34 [M13FSP6]: 5′-GGATCCATTTAGGTGACACTATAGAAGCCCAGTCACGACGTTGTAAAACGACGGC-3′ and SEQ ID NO: 35 [M13RT3]: 5′-CGGCCGAATTACCCTCACTAAAGGGACGGATAACA ATTTCACACAGGAAACAGC-3′) used in the amplification flank the NOV3 insert and bind to the M13 forward and reverse sequencing primer sites. M13FSP6 (SEQ ID NO: 34) and M13RT3 (SEQ ID NO: 35) contain promoters for SP6 and T3 RNA polymerases, respectively. The PCR mix contained 1 ng plasmid DNA, 0.2 uM M13FSP6, 0.2 μM M13RT3, 200 mM dNTPs, 0.5 μl Advantage cDNA polymerase mix (50×; Clontech) in 1× PCR buffer (Advantage cDNA Polymerase Kit, Clontech). The PCR cycling parameters were as follows: 94° C., 2 min (1 cycle); 94° C., 5 sec, 72° C., 5 min (5 cycles); 94° C., 5 sec, 70° C., 3 min (5 cycles); 94° C., 5 sec, 68° C., 3 min (15 cycles). Following amplification, the PCR product containing the gene fragment of interest was electrophoresed through a 1% low melt agarose gel and purified using the Qiaex II gel extraction kit (Qiagen).

An antisense RNA probe was generated from the PCR product using the Stip-EZ RNA probe synthesis kit (Ambion, Inc.) according to manufacturer's instructions. One hundred ng of PCR product was labeled with 25 μCi³³P-UTP (3 mM; Amersham) in a synthesis reaction using SP6 RNA polymerase. Following RNA transcription, 1 μl DNase I was added and the reaction was allowed to proceed for 15 minutes at 37° C. The unincorporated nucleotides were removed with ProbeQuant G-50 micro columns (Pharmacia Biotech) according to manufacturer's instructions. The probe was quantitated with a Bioscan QC-4000 according to manufacturer's instructions (Bioscan).

2.2. Hybridization.

The RNA probe was hybridized to four commercially available Northern Blots (Clontech) at 65° C. in a Robbins Scientific Model 400 hybridization incubator. Briefly, the blots were inserted into 15×300 mm glass tubes and prehybridized at 65° C. in 10 ml Zip-Hyb (Ambion, Inc.) for 30 min. The RNA probe (1.0×10⁶ dpm/ml) was added to 1.0 ml 65° C. Zip-Hyb and placed in the glass tube with the prehybridized Northern blots. Hybridization of the probe was allowed to proceed for 2 hours. Following hybridization, the buffer was removed and the blots were washed twice for 15 min in the glass tubes at 65° C. The first wash was with prewarmed (65° C.) 2× SSC, 0.1% SDS, while the second wash was with prewarmed 0.1× SSC, 0.1% SDS. The blots were removed from the glass tubes, wrapped in Saran Wrap and exposed to phosphor screens overnight (Molecular Dynamics). The phosphor screens were scanned the following day on a Molecular Dynamics Storm 840 at 50 micron resolution.

### Example 3

Mapping of Human NOV3

3.1. Oligonucleotide Design and Synthesis

A primer pair (SEQ ID NO:36-5′ GATTTCTTCTTGCTTTTGACTC 3′, SEQ ID NO:37-5′ ACTCAGCTGTTTTATGGTGG 3′) was designed (Primer 3 primer selection software package) to amplify a segment of the NOV3 gene. Oligonucleotides were synthesized by Integrated DNA Technologies, (Coralville, Iowa).

3.2. PCR, Electrophoresis and Imaging Conditions

PCR was performed using the GeneBridge 4 human radiation hybrid panel (Research Genetics Inc., Huntsville, Ala.) as template. In addition to the 93 hybrids in the mapping panel, hamster and human genomic DNA were used as controls. DNA from the RH cell lines (50 ng) was amplified in 10 μl reactions containing 4.5 pmole primers, 40 μM dNTPs, 10% Rediload (Research Genetics, Inc., Huntsville, Ala.) and 1/3 × concentration of Advantage cDNA polymerase mix (Clontech, Inc, Palo Alto, Calif.). PCR was performed using a Tetrad thermocycler in an oil-free system (MJ Research) with the following “touchdown” PCR profile: 3 min, 94° C. (1 cycle), 94° C., 30 sec, 67° C., 30 sec, 68° C., 30 sec, (2 cycles); 94° C., 30 sec, 65° C., 30 sec, 68° C., 30 sec (2 cycles); 94° C., 30 sec, 67° C., 30 sec (31 cycles).

Samples were electrophoresed on a 3% agarose gel (1× TBE) containing 0.5 μg/ml ethidium bromide and imaged using the AlphImager 950 still video system (Alpha Innotech, San Leandro, Calif.). The collective set of scores (0=no amplification; 1=amplification; 2=uncertain) for a single marker is called an RH vector. The NOV3 marker was assayed in duplicate to reduce errors, and a consensus was generated from the duplicate vectors.

Chromosomal placement of the human NOV3 gene was accomplished using information from the Whitehead Institute/Massachusetts Institute of Technology Center for At Genome Research radiation hybrid mapping website.

3.3. Results

The human NOV3 gene maps onto Human chromosome 3 at a LOD score of >22. The exact placement is at 3q21, 1.6 centiRay (cR) below D3S1576 and 4.6 cR above WI-3522 (One cR is the distance between markers at which there is a 1% probability of breakage).

### Example 4

Mammaglobin (MammX) Expression in Kidney 293 Cells

4.1. Cloning of MammX cDNA for expression in kidney 293 cells.

Based on the predicted reading frame, we designed PCR primers to amplify the coding region of hMammX. The forward primer was SEQ ID NO: 38 (5′-GGATCCACCATGAAGCTGCTGATGGTCCTCATGCTG-3′), and the reverse primer was SEQ ID NO: 39 (5′-CTCGAGATTACTCTTCATATTACACCAAATGCT-3′). PCR was initiated by heating 25 μl Mix 1 (75 pmoles primers, 4 μg adult bone marrow cDNA, 5 μmoles dNTPs) and 25 μl Mix 2 [1 unit Fidelity Expand polymerase (Boehringer Mannheim), 5 μl 10× Fidelity Expand Buffer (Boehringer Mannheim)] separately at 96° C. for 20 seconds. Mixes 1 and 2 were then pooled and the following PCR cycling parameters were used: 96° C., 3 min (1 cycle); 96° C., 30 sec, 55° C., 1 min, 68° C., 2 min (10 cycles); 96° C., 30 sec, 60° C., 1 min, 68° C., 2 min (20 cycles); 72° C., 7 min (1 cycle). After PCR, a single DNA fragment of approximately 0.3 kb was obtained. The DNA fragment was cloned into the pcDNA3.1 V5His TOPO vector (Invitrogen, Carlsbad, Calif.). The MammX insert was verified by DNA sequence analysis. The resulting expression vector, pcDNA3.1V5HisMammX was used for transient protein expression in mammalian kidney 293 cells.

4.2. Expression of hMammX in human embryonic kidney 293 cells.

The pcDNA3.1V5HisMammX vector was transfected into 293 cells using the LipofectaminePlus reagent following the manufacturer's instructions (Gibco/BRL). The cell pellet and supernatant were harvested 72 hours after transfection and examined for hMammX expression by Western blotting (reducing conditions) with an anti-V5 antibody. The hMammX protein was detected as a 10-kDa protein in the cell pellet. No secreted form of MammX was detected.

### Example 5

Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional., gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations. See Richard A. Shimkets et al., (1999) “Gene Expression Analysis by Transcript Profiling Coupled to a Gene Dtabase Query.” NATURE BIOTECHNOLOGY 17: 798-803.

3. PathCalling™ Technology:

The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E.coli into a CuraGen Corporation proprietary yeast strain (disclosed in U. S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2. 1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2. 1 vector from Invitrogen to provide clones used for expression and screening purposes.

NOV1 Analysis

The sequence of Acc. No. CG51689-02 was derived by laboratory cloning of cDNA fragments, by in silico prediction of the sequence. cDNA fragments covering either the full length of the DNA sequence, or part of the sequence, or both, were cloned. In silico prediction was based on sequences available in Curagen's proprietary sequence databases or in the public human sequence databases, and provided either the full length DNA sequence, or some portion thereof.

Exon Linking: The cDNA coding for the CG51689-02 sequence was cloned by the polymerase chain reaction (PCR) using the primers:

| | |
|---|---|
| 5′-TAGCGTTGGACCAGTCTCCTAAGATG-3′ and | (SEQ ID NO:40) |
| 5′-TTTAACTGCACAAAGGCTGTATTGCAG-3′. | (SEQ ID NO:41) |

Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain-amygdala, brain-cerebellum, brain-hippocampus, brain-substantia nigra, brain-thalamus, brain -whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea and uterus.

Physical clone: The PCR product derived by exon linking, covering the entire open reading frame, was cloned into the pCR2.1 vector from Invitrogen to provide clone 2572195_{—}0_{—}17.698010.J1.

The DNA sequence and protein sequence for a novel Syncollin-like gene were obtained by exon linking and are reported here as CuraGen Acc. No. CG51689-02.

Tissue expression

The Syncollin-like gene disclosed in this invention is expressed in at least the following tissues: exocrine tissues including duodenum, pancreas, and secretary granules of parotid gland. Expression information was derived from the tissue sources of the sequences that were included in the derivation of the sequence of CuraGen Acc. No. CG51689-02. The sequence is predicted to be expressed in the following tissues because of the expression pattern of (GENBANK-ID: gb:GENBANK-ID:AF008197|acc:AF008197.1) a closely related Rattus norvegicus syncollin mRNA, complete cds homolog in species Rattus norvegicus: duodenum, pancreas, and secretary granules of parotid gland.

NOV3

Exon Linking: The cDNA coding for the CG52234-02 sequence was cloned by the polymerase chain reaction (PCR) using the primers:

| | |
|---|---|
| 5′-CAGAGTCTTGCTCTGTCTCC-3′ and | (SEQ ID NO:42) |
| 5′-ATCTATATGAGGAGGGAGGCC-3′. | (SEQ ID NO:43) |

Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea and uterus.

Physical clone: The PCR product derived by exon linking, covering the entire open reading frame, was cloned into the pCR2.1 vector from Invitrogen to provide clone 10327789_{—}0_{—}16.698004.J10.

The DNA sequence and protein sequence for a novel Claudin 18-like gene were obtained by exon linking and are reported here as CuraGen Acc. No. CG52234-02. Clone 3224646 was isolated from human testis.

Tissue expression

The Claudin 18-like gene disclosed in this invention is expressed in at least the following tissues: Brain, Lung, Whole Organism. Expression information was derived from the tissue sources of the sequences that were included in the derivation of the sequence of CuraGen Acc. No. CG52234-02. The sequence is predicted to be expressed in the following tissues because of the expression pattern of (GENBANK-ID: gb:GENBANK-ID:AF221069|acc:AF221069.1) a closely related Homo sapiens Claudin-18 mRNA, complete cds homolog in species Homo sapiens: tight junctions.

The nucleic acids and proteins of the invention have applications in the diagnosis and/or treatment of various diseases and disorders. For example, the compositions of the present invention will have efficacy for the treatment of patients suffering from: Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, systemic lupus erythematosus, autoimmune disease, asthma, emphysema, scleroderma, allergies, ARDS, cancer, as well as other diseases, disorders and conditions.

### Example 6

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM™ 7700 or an ABI PRISMS™ 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28S: 18S) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75bp to 100bp. The probes and primers selected (see below) were synthesized by Synthegen (Houston, Tex., USA). Probes were double purified by HPLC to remove uncoupled dye and evaluated by mass spectroscopy to verify coupling of 10 reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue . and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵cells/ml for 8 days, changing the media every 3 days and adjusting the cell concentration to 5×10⁵cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

NOV4

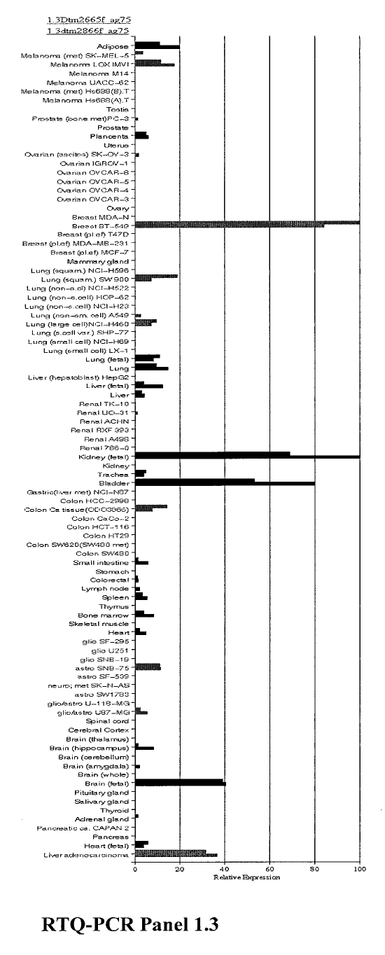

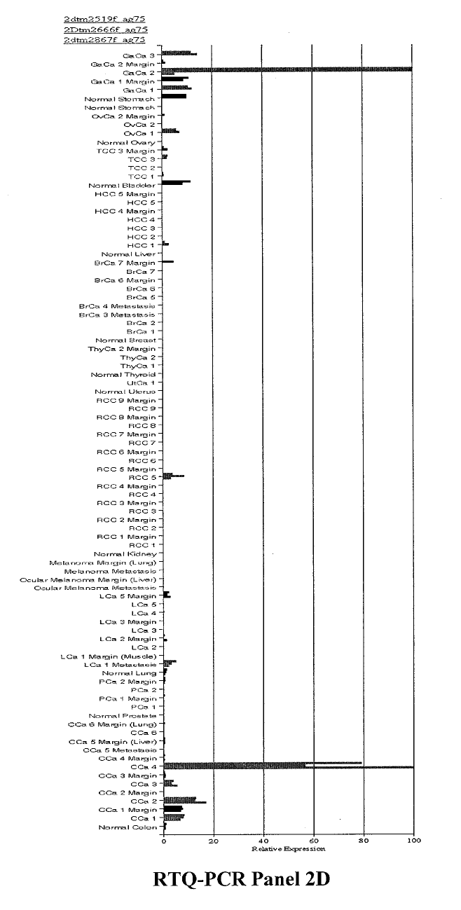

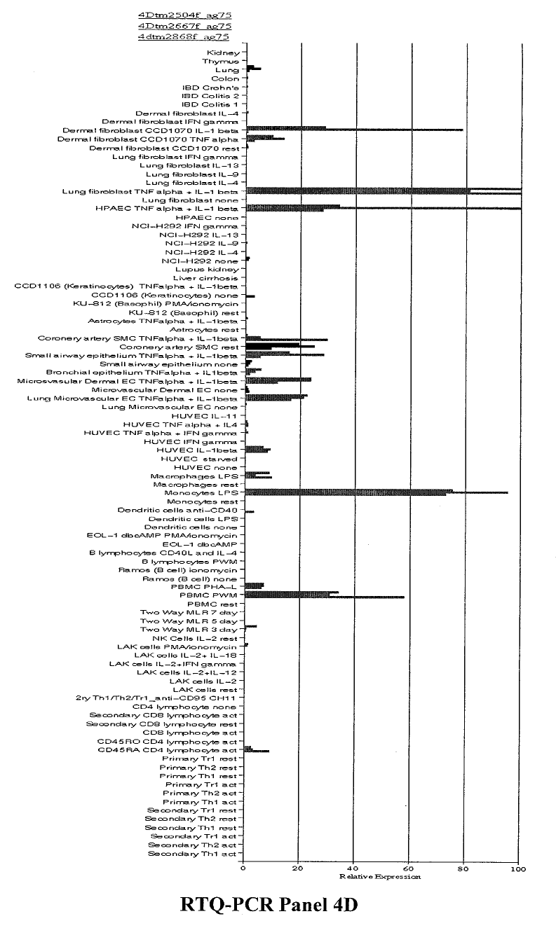

TaqMan Expression profile of NOV4b-CG54942-01 are shown in FIGS 1A-1C.

Panel 1.3D (FIG. 1A): In adult tissues, the highest level of expression of CG54942-01 is in the bladder and in a breast cancer. Most normal tissues including lymphoid and respiratory tract tissues express very low levels of this antigen (B7-549).

Panel 2D (FIG. 1B): CG54942-01 is expressed consistently in colon cancers (CCa4) but not in normal colon.

Panel 4D (FIG. 1C): CG54942-01 is expressed in lung fibroblasts, small airway epithelium, and micro-vascular dermal EC after treatment with IL-1beta and TNFalpha, HPAEC and dermal fibroblasts after treatment with IL-1 beta, monocytes after LPS treatment and PBMC after treatment with poke weed mitogen.

Probe Name: Ag75

| | | | |
|---|---|---|---|
| Primers Sequences | TM | Length | SEQ ID NO: |

| | | | | |
|---|---|---|---|---|
| Forward | 5′-ACAAGCTTTCTTCCCATTCTTGAG-3′ | 62.2 | 24 | 44 |
| Probe | FAM-5′-TGCACCCAAACGGAAGTCATAGCCA-3′-TAMRA | 72.1 | 25 | 45 |
| Reverse | 5′-GAACGTGAAGTCCCGTGGAC-3′ | 62.9 | 20 | 46 |

Potential Role(s) of CG54942-01 in Inflammation: CG54942-01 is induced by the proinflammatory cytokine IL-1 and mitogens. The expression of the protein encoded by CG54942-01 may contribute to inflammation by inducing the extravasation of inflammatory cells, activating leukocytes, or inducing the production of proinflammatory cytokines.

Impact of Therapeutic Targeting of CG54942-01: Targeting of the protein encoded for by this transcript with antibody or small molecule therapeutics could reduce or inhibit inflammation in the lung due to asthma/allergy, emphysema and viral or bacterial infections. Such therapeutics could also reduce inflammation in the skin due to psoriasis, DTH, and viral or bacterial infections.

### Example 7

Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

Three polymorphic variants of NOV1c have been identified and are shown in Table D1.

TABLE D1

| | Nucleotides | | |
|---|---|---|---|
| Variant No. | Base Position of SNP | Wild-type | Variant |

| | | |
|---|---|---|
| 190 | C | T |
| 344 | T | C |
| 642 | A | G |

Northern analysis showed expression of NOV3 in a variety of human fetal and adult tissues. The human NOV3 maps onto human chromosome 3q21, 1.6 centiRay below D3S2576 and 4.6 cR above WI-3522.

The nucleic acids and proteins of the invention have applications in the diagnosis and/or treatment of various diseases and disorders. For example, the compositions of the present invention will have efficacy for the treatment of patients suffering from: Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, systemic lupus erythematosus, autoimmune disease, asthma, emphysema, scleroderma, allergies, ARDS, cancer, as well as other diseases, disorders and conditions.

### EQUIVALENTS

From the foregoing detailed description of the specific embodiments of the invention, it should be apparent that particular novel compositions and methods involving the coding nucleic acids, the polypeptides, detection and treatment methods have been described. Although these particular embodiments have been disclosed herein in detail, this has been done by way of example for purposes of illustration only, and is not intended to be limiting with respect to the scope of the appended claims which follow. In particular, it is contemplated by the inventors that various substitutions, alterations, and modifications may be made as a matter of routine for a person of ordinary skill in the art to the invention without departing from the spirit and scope of the invention as defined by the claims.

What is claimed is:

1. An isolated nucleic acid molecule comprising a nucleotide sequence at least 85% homologous to a nucleotide sequence selected from the group consisting of SEQ ID NOs: 1, 3, 5, 7, 9, 11, 13, 14 and 16, or a complement thereof.

2. The nucleic acid molecule of claim 1, wherein the nucleic acid molecule is at least 90% homologous to the nucleotide sequence, or a complement thereof.

3. The nucleic acid molecule of claim 1, wherein the nucleic acid molecule is at least 95% homologous to the nucleotide sequence, or a complement thereof.

4. The nucleic acid molecule of claim 1, wherein the nucleic acid molecule is at least 98% homologous to the nucleotide sequence, or a complement thereof.

5. The nucleic acid molecule of claim 1, wherein the nucleic acid molecule encodes a polypeptide that binds to syntaxin, SNAP, SNAP-25, synaptobrevin, synaptogamin or the N-type calcium channel.

6. The nucleic acid molecule of claim 1, wherein the nucleic acid encodes a polypeptide comprising at least one of the amino acid sequences of SEQ ID NO: 1, SEQ ID NO: 3 and SEQ ID NO: 5.

7. The nucleic acid molecule of claim 1, wherein the nucleic acid molecule encodes a polypeptide comprising the amino acid sequence of SEQ ID NO: 7.

8. The nucleic acid molecule of claim 1, wherein the nucleic acid molecule comprises any one of SEQ ID NO: 9, SEQ ID NO: 11 and SEQ ID NO: 13, or a complement thereof.

9. The nucleic acid molecule of claim 1, wherein the nucleic acid molecule comprises any one of SEQ ID NO: 14 and SEQ ID NO: 16, or a complement thereof.

10. The nucleic acid molecule of claim 1, wherein the nucleic acid molecule encodes a polypeptide reactive with an anti-claudin antibody.

11. The nucleic acid molecule of claim 1, wherein the nucleic acid molecule encodes a polypeptide comprising the amino acid sequence of any one of SEQ ID NO: 1, 3 and 5.

12. The nucleic acid molecule of claim 1, wherein the nucleic acid molecule comprises any one of SEQ ID NO: 9 and SEQ ID NO: 11, or a complement thereof as shown in SEQ ID NO: 13.

13. The nucleic acid molecule of claim 1, wherein the nucleic acid molecule encodes a polypeptide having T cell proliferation activity.

14. The nucleic acid molecule of claim 1, wherein the nucleic acid molecule encodes a polypeptide comprising any one of the amino acid sequence of SEQ ID NO: 15 and 17.

15. The nucleic acid molecule of claim 1, wherein the nucleic acid molecule comprises the sequence of SEQ ID NO: 7.

16. A vector comprising the nucleic acid molecule of claim 1.

17. A cell comprising the vector of claim 16.

18. An isolated polypeptide selected from the group consisting of:
a) a polypeptide at least 80% homologous to any one of the amino acid sequences of SEQ ID NO: 2, 4 and 6;
b) a polypeptide at least 80% homologous to the amino acid sequence of SEQ ID NO: 8;
c) a polypeptide at least 60% homologous to any one of the amino acid sequences of SEQ ID NO: 10 and 12;
d) a polypeptide at least 80% homologous to any one of the amino acid sequences of SEQ ID NO: 15 and 17;
e) a fragment of a polypeptide comprising any one of the amino acid sequences of SEQ ID NOs: 2, 4, 6, 8, 10, 12, 15 and 17, wherein the fragment comprises at least 6 amino acids of any one of SEQ ID NOs: 2, 4, 6, 8, 10, 12, 15 and 17; and
f) a naturally occurring allelic variant consisting of any one of the amino acid sequence of SEQ ID NOs: 2, 4, 6, 8, 10, 12, 15 and 17, wherein the polypeptide is encoded by a nucleic acid molecule that hybridizes under stringent conditions to a nucleic acid molecule consisting of any one of SEQ ID NOs: 1, 3, 5, 7, 9, 11, 13, 14 and 16.

19. The polypeptide of claim 18, wherein the polypeptide, or fragment thereof, has an activity selected from the group consisting of:
a) a syncline-like activity, wherein the activity is modulated by the binding and release of calcium ions;
b) a claudin-like activity; and
c) a cytokine-like activity.

20. The polypeptide of claim 18, wherein the polypeptide comprises the amino acid sequence of any one of SEQ ID NO: 2, 4, 6, 8, 10, 12, 15 and 17.

21. The polypeptide of claim 19, wherein the polypeptide consists of the amino acid sequence of any one of SEQ ID NO: 2, 4, 6, 8, 10, 12, 15 and 17.

22. An antibody which selectively binds to a polypeptide of claim 18.

23. An antibody which selectively binds to a polypeptide comprising the amino acid sequence of any one of SEQ ID NO: 2, 4, 6, 8, 10, 12, 15 and 17.

24. A method for producing the polypeptide of claim 18, the method comprising culturing the host cell of claim 17 under conditions in which the nucleic acid molecule is expressed.

25. A method for detecting the presence of a polypeptide in a sample from a mammal, the method comprising:
a) contacting a sample suspected of containing the polypeptide with an antibody of claim 22 or 34 that binds to the polypeptide under conditions which allow for formation of complexes comprising the antibody and polypeptide; and
b) detecting the formation of reaction complexes comprising the antibody and the polypeptide in the sample, wherein detection of the formation of reaction complexes indicates the presence of the polypeptide in the sample.

26. The method of claim 25, wherein the mammal is a human.

27. A method for detecting or diagnosing the presence of a disease associated with altered levels of a polypeptide having an amino acid sequence at least 80% identical to a polypeptide with an amino acid sequence selected from the group consisting of SEQ ID NOs: 2, 4, 6, 8, 10, 12, 15 and 17 in a sample, the method comprising:
a) measuring the level of the polypeptide in a biological sample from the mammalian subject according to claim 25; and
b) comparing the level detected in step a) to a level of the polypeptide present in normal subjects, or in the same subject at a different time, in which an increase or decrease in the level of the polypeptide as compared to normal levels indicates a disease condition.

28. A method of detecting the presence of a nucleic acid molecule having a sequence at least 80% identical to a nucleic acid comprising a sequence selected from the group consisting of SEQ ID NOs: 1, 3, 5, 7, 9, 11, 13, 14 and 16, or a complement thereof, in a sample from a mammal, the method comprising:
a) contacting the sample with a nucleic acid probe or primer which selectively hybridizes to the nucleic acid molecule; and
b) determining whether the nucleic acid probe or primer binds to a nucleic acid molecule in the sample,
wherein binding of the nucleic acid probe or primer indicates the nucleic acid molecule is present in the sample.

29. The method of claim 28, wherein the mammal is a human.

30. A method for detecting or diagnosing the presence of a disease associated with altered levels of a nucleic acid at least 80% identical to a nucleic acid comprising a sequence selected from the group consisting of SEQ ID NOs: 1, 3, 5, 7, 9, 11, 13, 14 and 16, or a complement thereof, in a sample from a mammal., the method comprising:
a) measuring the level of the nucleic acid in a biological sample from the mammalian subject according to claim 28; and
b) comparing the level detected in step a) to a level of the nucleic acid present in normal subjects, or in the same subject at a different time, in which an increase or decrease in the level of the nucleic acid as compared to normal levels indicates a disease condition.

31. A method of treating a pathological state in a mammal., the method comprising administering to the subject a polypeptide to the subject in an amount to alleviate the pathological condition, wherein the polypeptide a polypeptide having an amino acid sequence at least 80% identical to a polypeptide with an amino acid sequence selected from the group consisting of SEQ ID NOs: 2, 4, 6, 8, 10, 12, 15 and 17, or a biologically active fragment thereof.

32. The method of claim 31, wherein the mammal is a human.

33. A method of treating a pathological state in a mammal, the method comprising administering to the subject the antibody of claim 22 or 23 in an amount to alleviate the pathological condition.

34. The method of claim 33, wherein the mammal is a human.
